(19) United States
(12) Patent Application Publication
(10) Pub. No.: US 2022/0233583 A1
(43) Pub. Date: Jul. 28, 2022

(54) METHOD FOR TREATING OR PREVENTING SACCHARIDE-RELATED DISEASES OR DISORDERS

(71) Applicant: SHANGHAI JIAOTONG UNIVERSITY, Shanghai (CN)

(72) Inventors: Shiyi ZHANG, Shanghai (CN); Xin ZHAO, Shanghai (CN); Meng TIAN, Shanghai (CN); Bingxue YANG, Shanghai (CN); Songxuan SUN, Shanghai (CN); Huijun ZHANG, Shanghai (CN)

(21) Appl. No.: 17/616,628

(22) PCT Filed: Jun. 3, 2020
(86) PCT No.: PCT/CN2020/094198

§ 371 Date: Dec. 3, 2021

(30) Foreign Application Priority Data
Jun. 6, 2019 (CN) 201910492851.5

(51) Int. Cl. A61K 31/80; A61P 3/10; A61K 9/00

(52) U.S. Cl. CPC A61K 31/80; A61K 9/0053; A61P 3/10

(57) ABSTRACT

A method for treating or preventing saccharide-related diseases or disorders, comprising administering a therapeutically or prophylactically effective amount of a polymer comprising at least one boronic acid group to a subject having or at risk of having a saccharide-related disease or disorder.

### TECHNICAL FIELD

The present application relates to the field of biomedicine, in particular to a method for treating or preventing saccharide-related diseases or disorders.

### BACKGROUND

When fasting (without any food intake other than water within 8 hours) blood glucose is higher than the normal range, it is called hyperglycemia. The normal value of fasting blood glucose is 3.9-5.6 mmol/L, and when the blood glucose two hours after a meal is higher than the normal range of 7.8 mmol/L, it can also be called hyperglycemia. When the concentration of glycosylated hemoglobin (HbA1c) is higher than 42 mmol/mol or the proportion of glycosylated hemoglobin is higher than 6.0%, it can also be called hyperglycemia. Elevated blood glucose may cause symptoms such as polyuria, thirst, and polydipsia, and sustained high blood glucose may cause damages to tissues and organs and increase the incidence of various serious diseases, such as cardiovascular disease, chronic kidney disease and the like. Saccharide-related diseases or disorders, such as obesity, diabetes, and fatty liver, seriously threaten human health.

### SUMMARY OF THE INVENTION

The present application provides a method for treating or preventing saccharide-related diseases or disorders, comprising administering a therapeutically or prophylactically effective amount of a polymer comprising at least one boronic acid group to a subject having or at risk of having the saccharide-related diseases or disorders.

The present application also provides a method for reducing a saccharide level of a subject, comprising administering a therapeutically or prophylactically effective amount of a polymer comprising at least one boronic acid group to the subject.

The present application also provides a method for preventing an increase in the saccharide level of a subject, comprising administering a therapeutically or prophylactically effective amount of a polymer comprising at least one boronic acid group to the subject.

The present application also provides a method for preventing or slowing down the absorption of saccharide by a subject, comprising administering a therapeutically or prophylactically effective amount of a polymer comprising at least one boronic acid group to the subject.

In some embodiments, wherein the saccharide level is the saccharide level of the subject after a meal.

In some embodiments, wherein the polymer comprising at least one boronic acid group is administered as a pharmaceutically active ingredient.

In some embodiments, wherein the pharmaceutical activity comprises that, compared with a control group, the proportion of the saccharide that has been degraded enzymatically in the subject to whom the polymer comprising at least one boronic acid group is administered is reduced, and the control group is the subject to whom the polymer comprising at least one boronic acid group is not administered.

In some embodiments, wherein the enzymatic hydrolysis comprises enzymatic hydrolysis by a related saccharidase, and the related saccharidase comprises a glycosylase.

In some embodiments, wherein the polymer comprising at least one boronic acid group interacts with the saccharide directly.

In some embodiments, wherein before, simultaneously and/or after the administration of the polymer comprising at least one boronic acid group, other hypoglycemic drugs are administered to the subject.

In some embodiments, wherein the other hypoglycemic drugs are selected from insulin and the analogue thereof, insulin secretagogue, metformin drug, α-glucosidase inhibitor, insulin sensitizer, peroxisome proliferator-activated receptor agonist (PPAR agonist), GPR40 agonist, JNK inhibitor, pan-AMPK activator, incretin analogue, glucokinase agonist (GKA), G protein-coupled receptor agonist (GPCR agonists), SGLT1 inhibitor, SGLT2 inhibitor, DPP-4 inhibitor, glucagon receptor agonist (GCGR agonist), GIP receptor agonist, GSK-3 inhibitor, amylin analogue, vanadium-containing compound, GFAT inhibitor, 11β-HSD1 inhibitor, Sirtuin-1 (SIRT-1) agonist, PTP1B inhibitor, PI3K agonist, GLP-2 receptor agonist, and/or GLP-1 receptor agonist.

In some embodiments, wherein the saccharides is selected from: monosaccharide, disaccharide, polysaccharide, and/or substance comprising the monosaccharide, the disaccharide and/or the polysaccharide.

In some embodiments, wherein the administration is oral administration.

In some embodiments, wherein the polymer comprising at least one boronic acid group is formulated as an oral preparation.

In some embodiments, wherein the saccharide-related diseases or disorders are selected from: obesity, diabetes and/or fatty liver.

In some embodiments, wherein the polymer comprising at least one boronic acid group has a structure represented by Formula I,

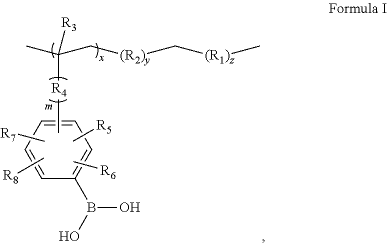

Wherein, R1 or R2 is selected from the following structures and salts thereof:

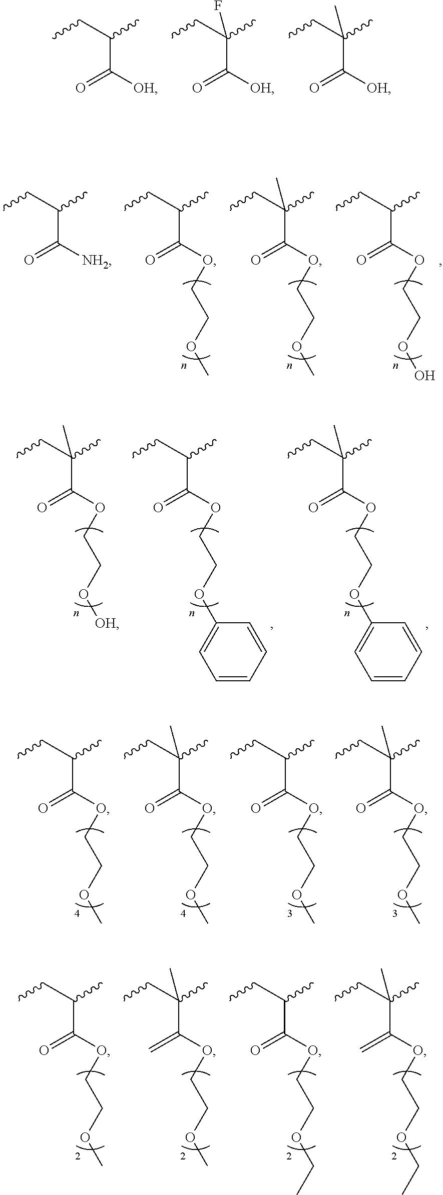

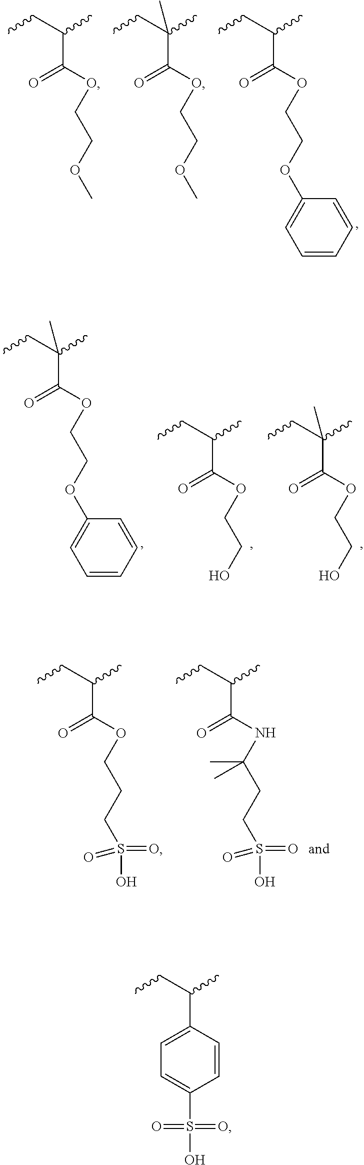

wherein n is an integer greater than or equal to 0;
R3 is selected from the following structures:

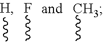

R4 is selected from the following structures:

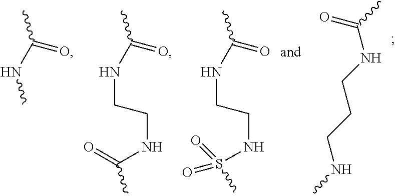

R5 or R6 or R7 or R8 is selected from the following structures:

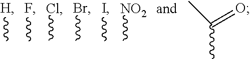

m is an integer greater than or equal to 0, x is a positive integer greater than or equal to 1, y and z are integers greater than or equal to 0, and when y is not equal to 0, x:y=1:(0.000001-90); when z is not equal to 0, x:z=1:(0.000001-90);
Alternatively, the polymer has a structure represented by Formula II,

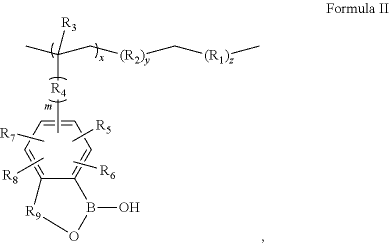

Wherein, R1 or R2 is selected from the following structures and salts thereof:

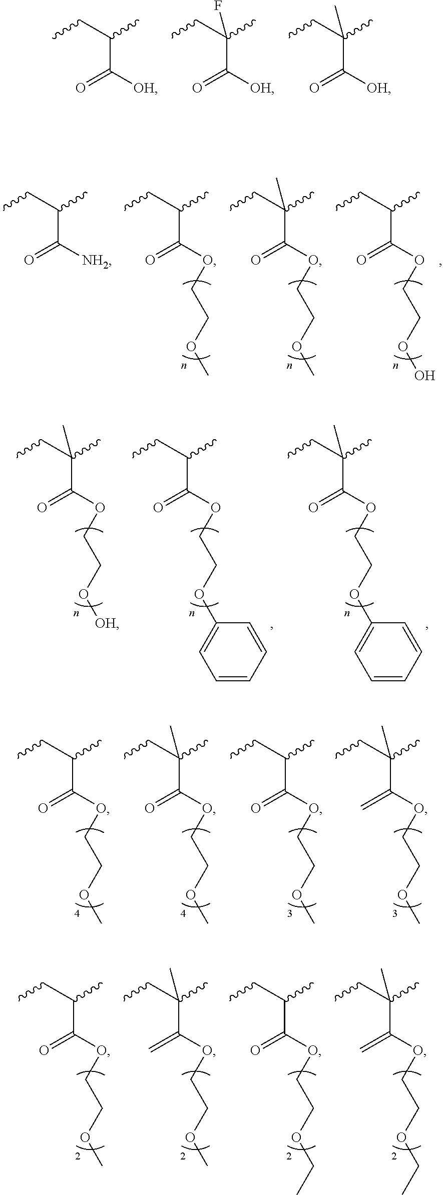

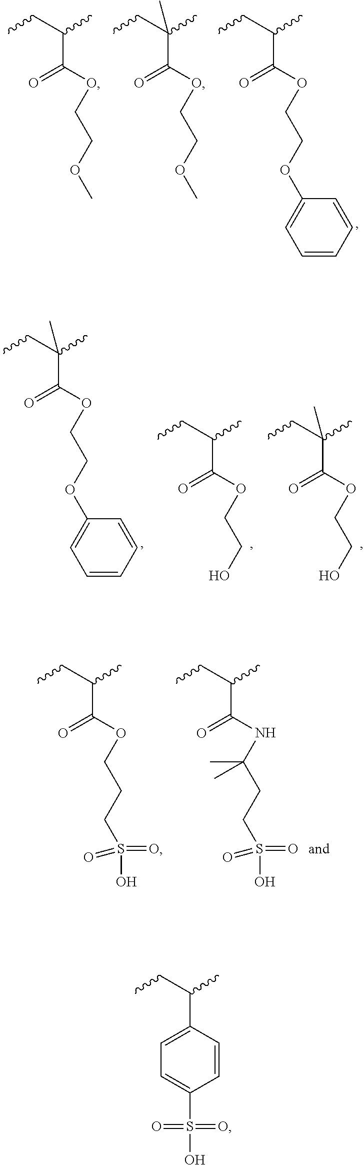

wherein n is an integer greater than or equal to 0;
R3 is selected from the following structures:

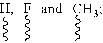

R4 is selected from the following structures:

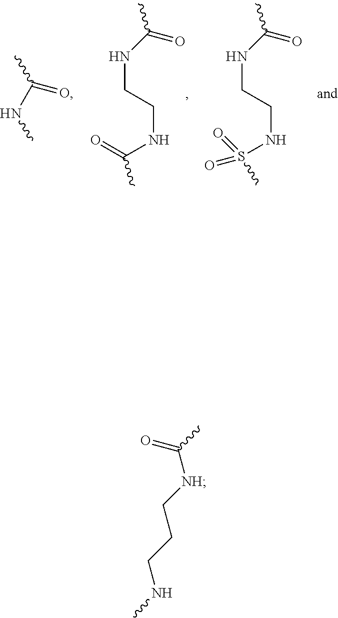

R5 or R6 or R7 or R8 is selected from the following structures:

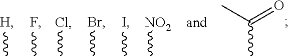

R9 is selected from the following structures:

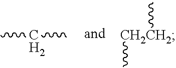

m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90);
Alternatively, the polymer has a structure represented by Formula III,

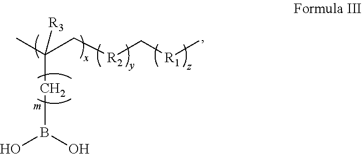

Wherein, R1 or R2 is selected from the following structures:

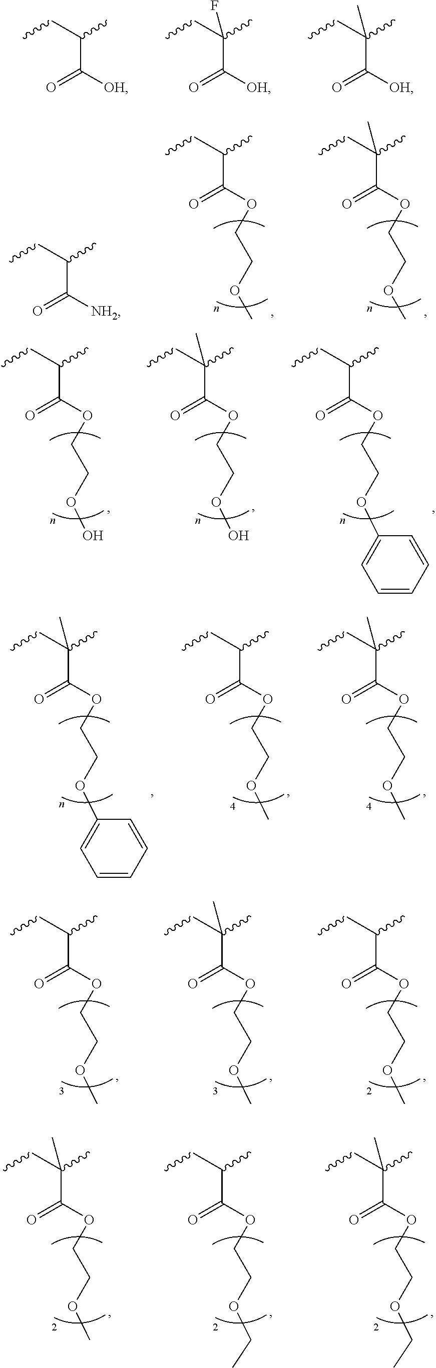

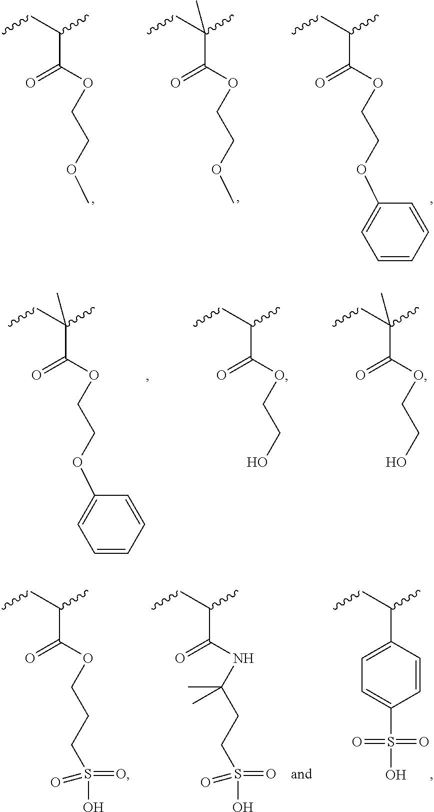

wherein n is an integer greater than or equal to 0;
R3 is selected from the following structures:

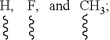

m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90).

In some embodiments, wherein the polymer comprising at least one boronic acid group is selected from:

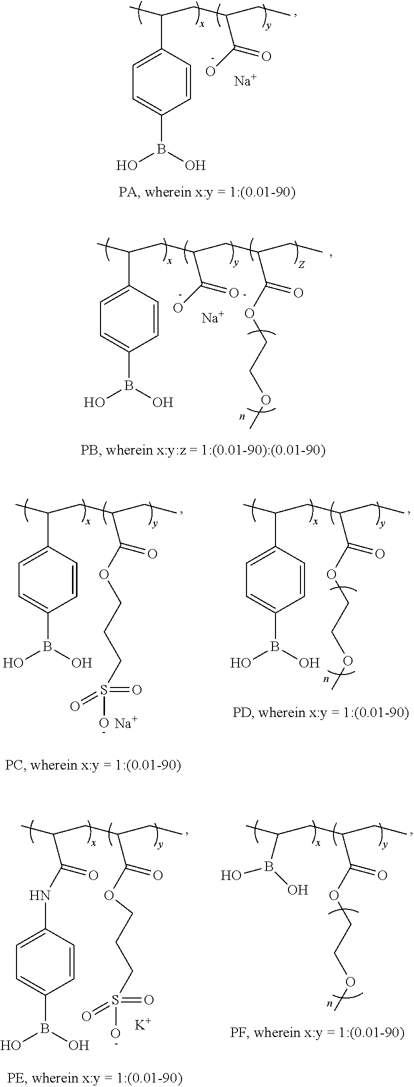

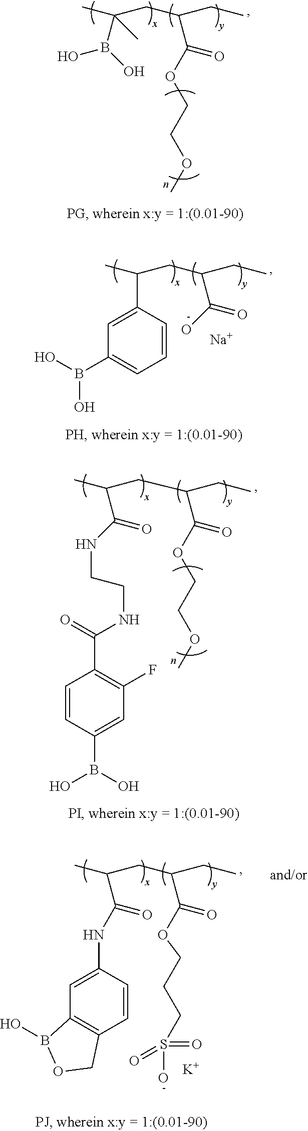

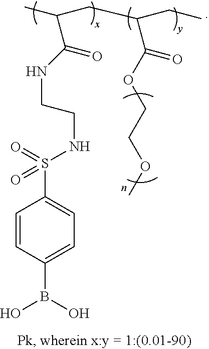

In some embodiments, wherein the polymer comprising at least one boronic acid group is selected from:

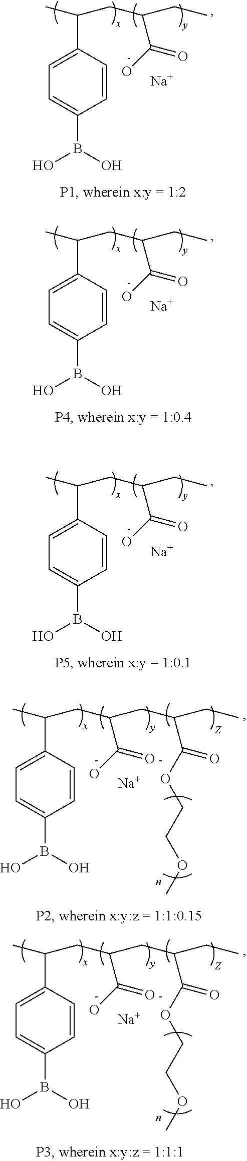

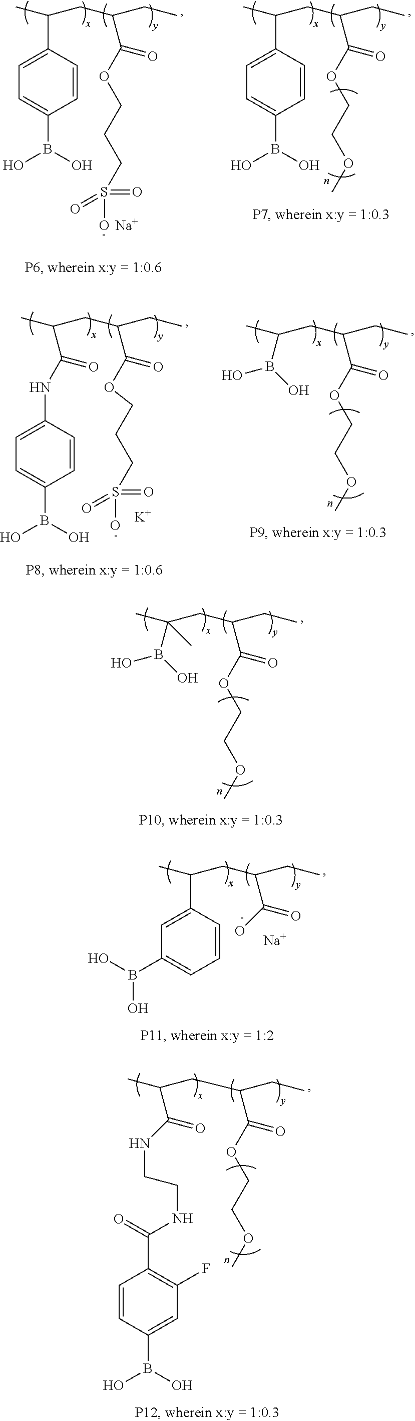

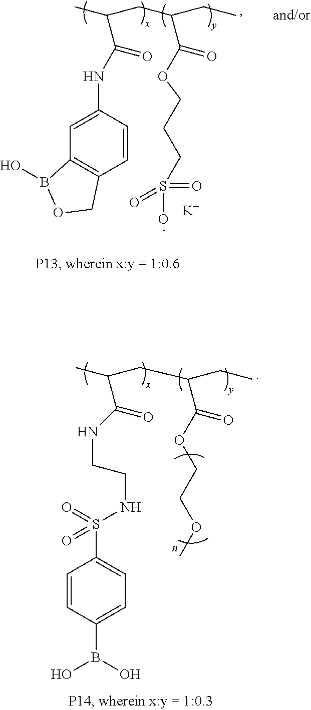

The present application also provides a use of a polymer comprising at least one boronic acid group in the preparation of a medicament, the medicament is used to treat or prevent saccharide-related diseases or disorders.

In some embodiments, according to the use of the present application, wherein the saccharide-related diseases or disorders comprise obesity, diabetes and/or fatty liver.

In some embodiments, according to the use of the present application, wherein the medicament is used to reduce the saccharide level.

In some embodiments, according to the use of the present application, wherein the medicament is used to prevent an increase of the saccharide level.

In some embodiments, according to the use of the present application, wherein the medicament is used to prevent or slow down the absorption of the saccharide.

In some embodiments, according to the use of the present application, wherein the saccharide comprises: monosaccharide, disaccharide, polysaccharide and/or substance comprising the monosaccharide, the disaccharide and/or the polysaccharide.

In some embodiments, according to the use of the present application, wherein the medicament comprises a therapeutically or prophylactically effective amount of the polymer comprising at least one boronic acid group as the therapeutically or prophylactically active ingredient of the medicament.

In some embodiments, according to the use of the present application, wherein the medicament is formulated as a preparation suitable for oral administration.

In some embodiments, according to the use of the present application, wherein the polymer has a structure represented by Formula I,

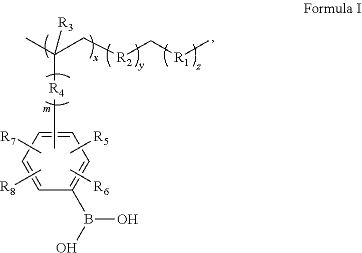

Wherein, R1 or R2 is selected from the following structures and salts thereof:

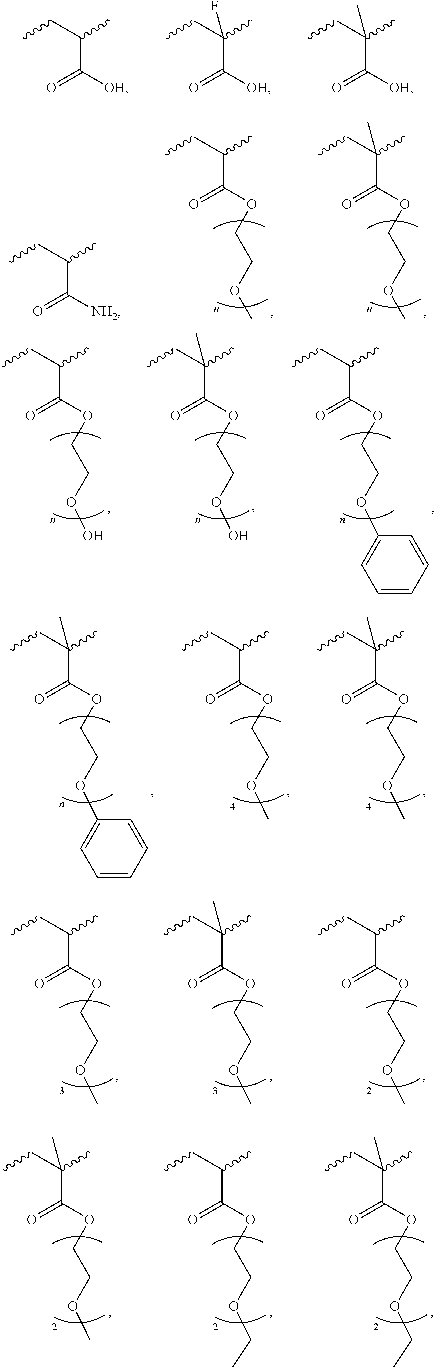

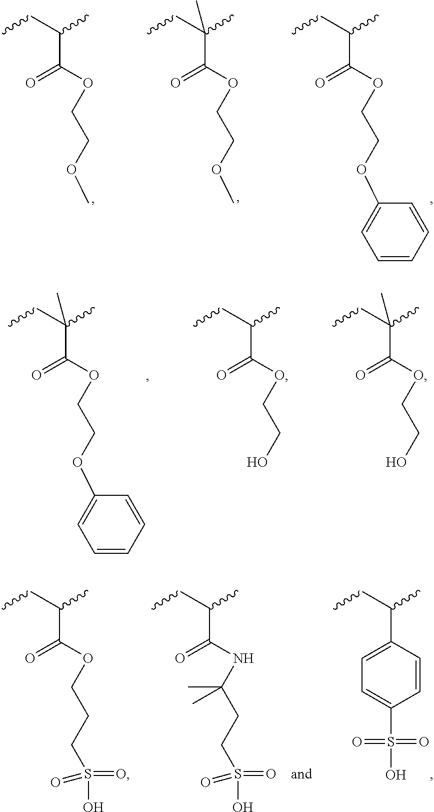

wherein n is an integer greater than or equal to 0;
R3 is selected from the following structures:

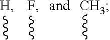

R4 is selected from the following structures:

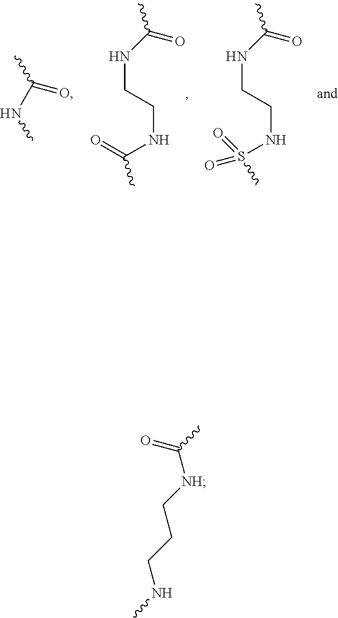

R5 or R6 or R7 or R8 is selected from the following structures:

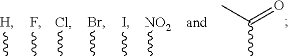

m is an integer greater than or equal to 0, x is a positive integer greater than or equal to 1, y and z are integers greater than or equal to 0, and when y is not equal to 0, x:y=1:(0.000001-90); when z is not equal to 0, x:z=1:(0.000001-90);
Alternatively, the polymer has a structure represented by Formula II,

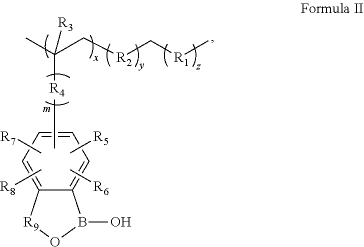

Wherein, R1 or R2 is selected from the following structures and salts thereof:

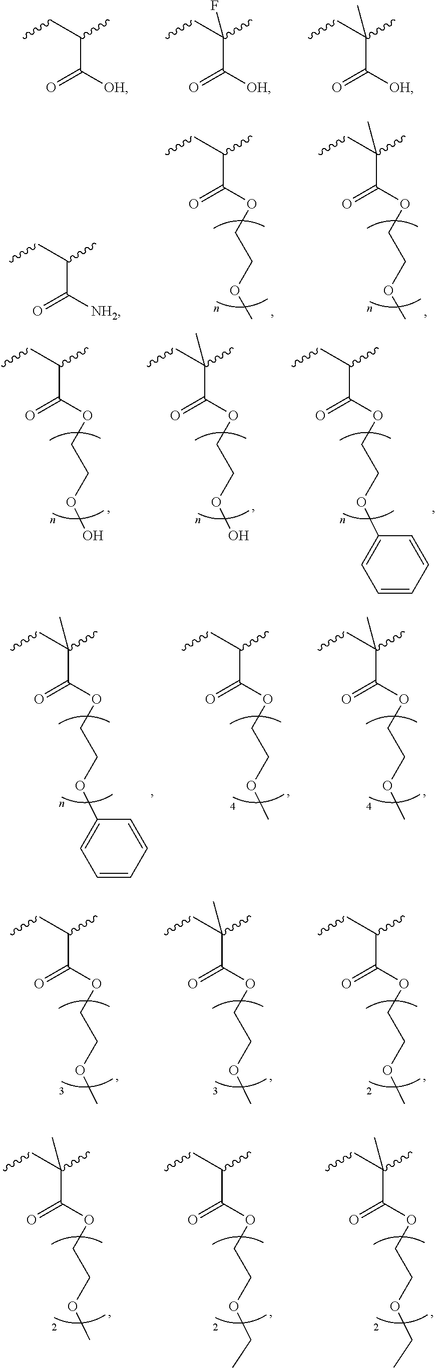

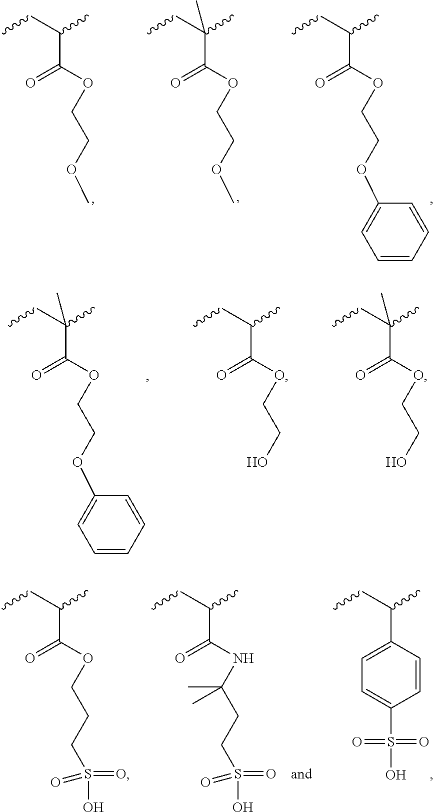

wherein n is an integer greater than or equal to 0;
R3 is selected from the following structures:

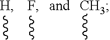

R4 is selected from the following structures:

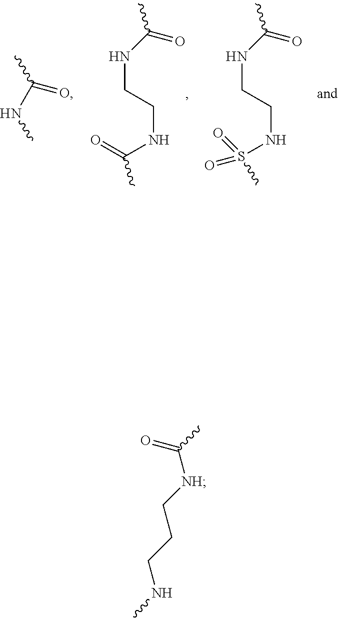

R5 or R6 or R7 or R8 is selected from the following structures:

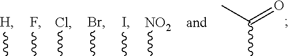

R9 is selected from the following structures:

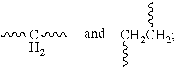

m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90);
Alternatively, the polymer has a structure represented by Formula III,

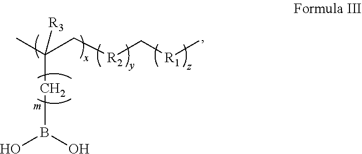

Wherein, R1 or R2 is selected from the following structures:

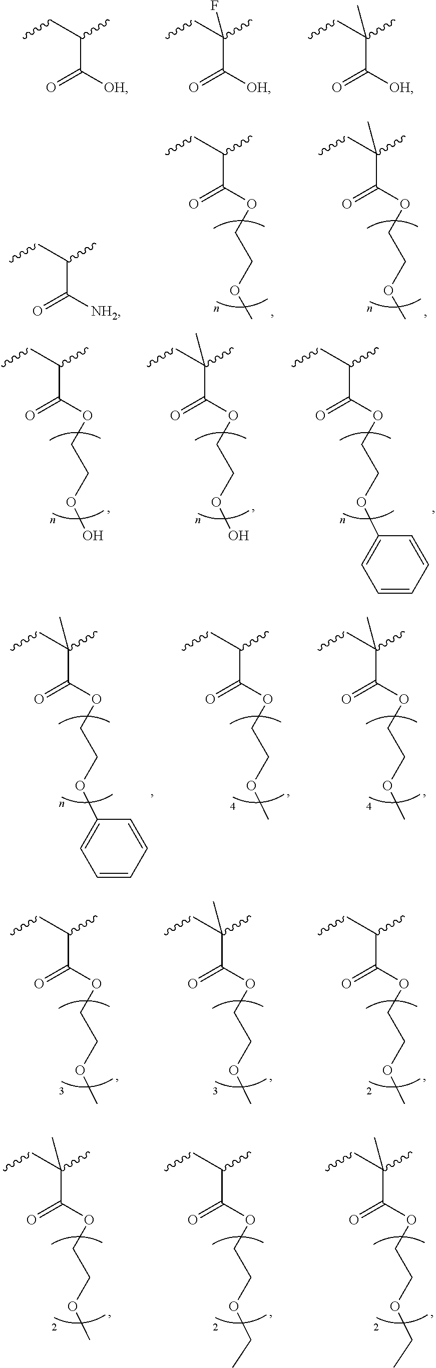

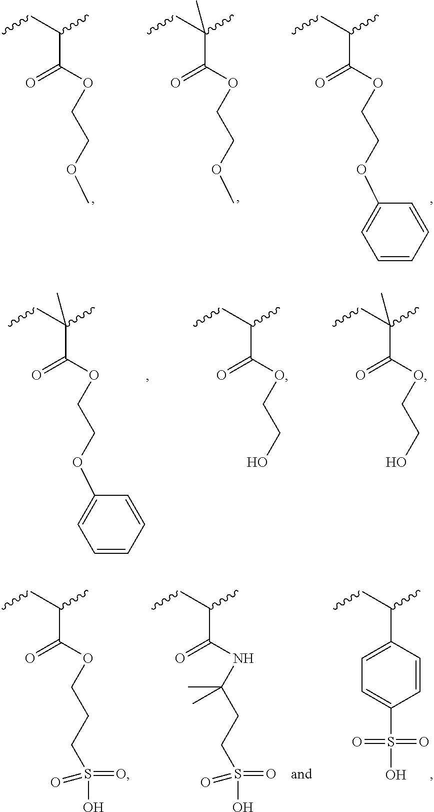

wherein n is an integer greater than or equal to 0;
R3 is selected from the following structures:

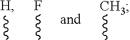

m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90).

In some embodiments, according to the use of the present application, wherein the polymer comprising at least one boronic acid group is selected from:

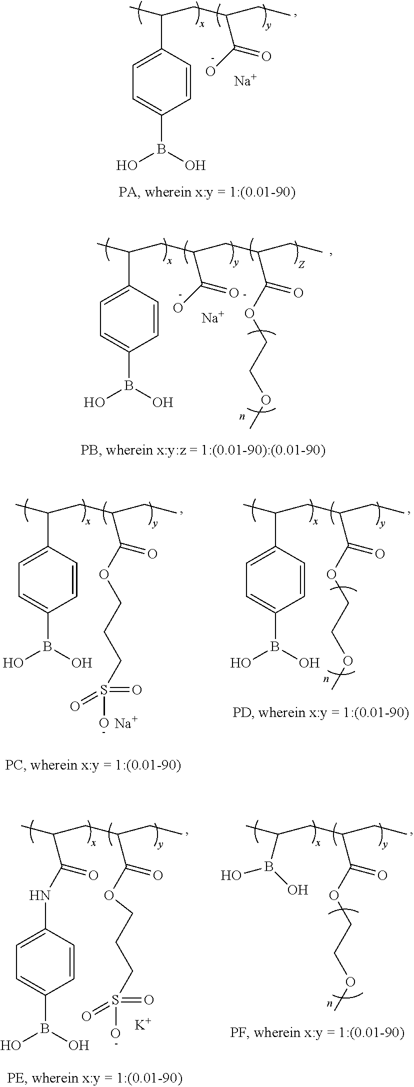

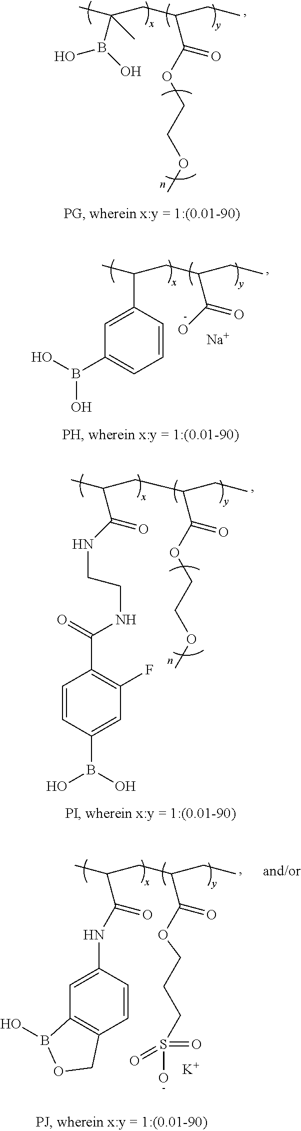

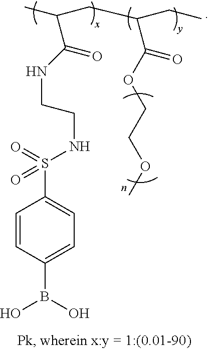

In some embodiments, according to the use of the present application, wherein the polymer is selected from:

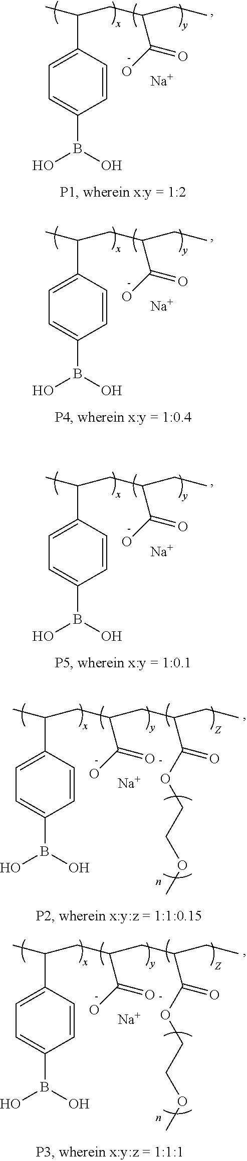

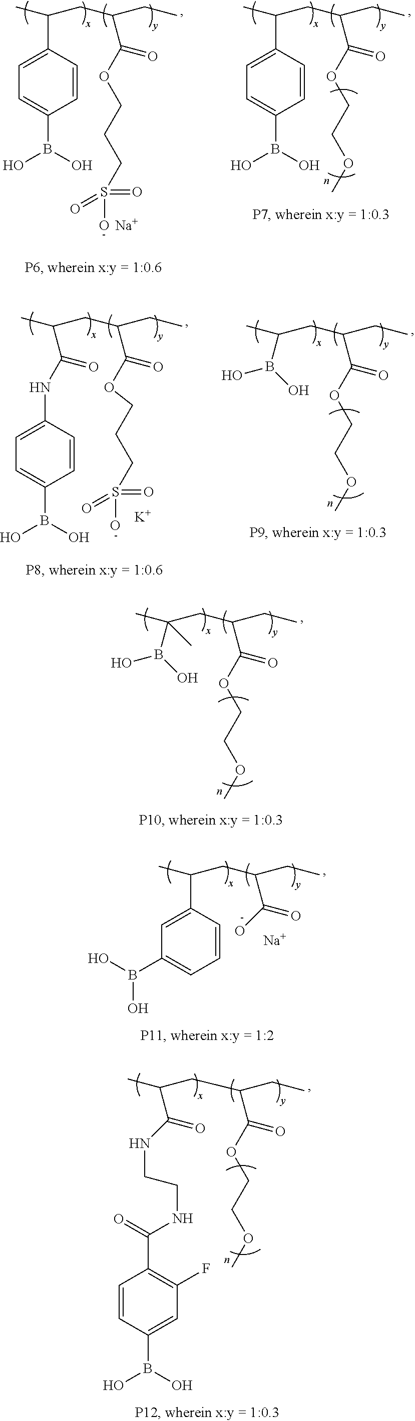

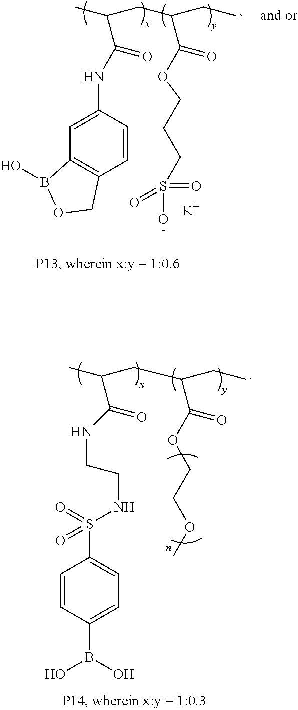

The present application also provides a pharmaceutical composition, the pharmaceutically active ingredient of which comprises a polymer comprising at least one boronic acid group.

In some embodiments, according to the pharmaceutical composition of the present application, wherein the polymer has a structure represented by Formula I,

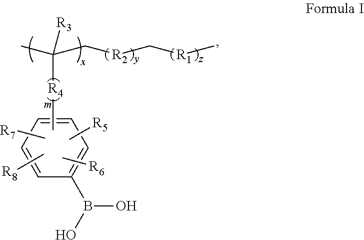

Wherein, R1 or R2 is selected from the following structures and salts thereof:

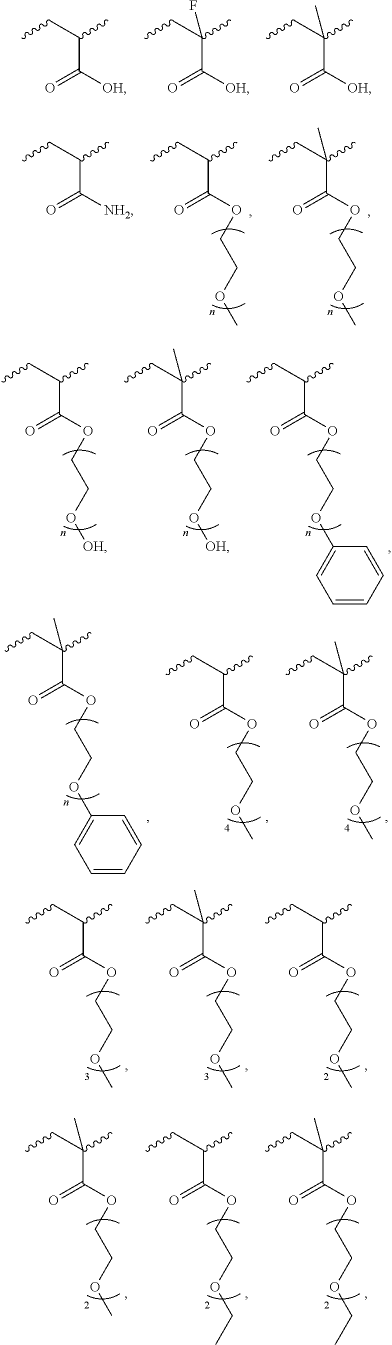

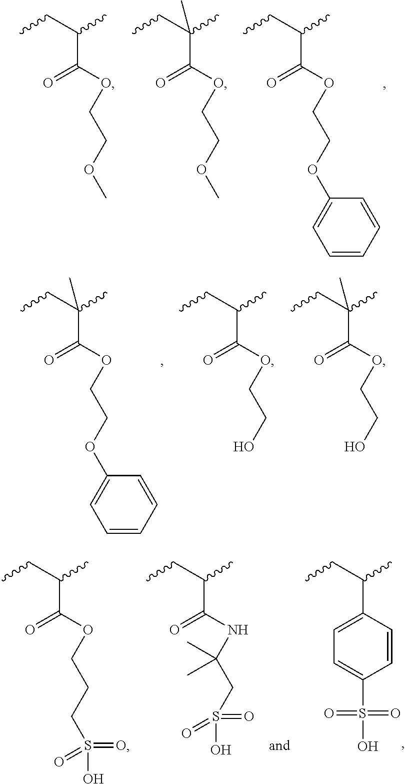

wherein n is an integer greater than or equal to 0;
R3 is selected from the following structures:

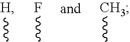

R4 is selected from the following structures:

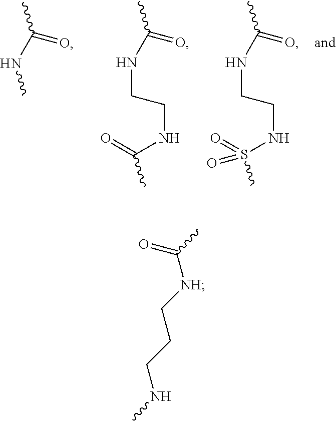

R5 or R6 or R7 or R8 is selected from the following structures:

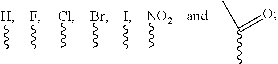

m is an integer greater than or equal to 0, x is a positive integer greater than or equal to 1, y and z are integers greater than or equal to 0, and when y is not equal to 0, x:y=1:(0.000001-90); when z is not equal to 0, x:z=1:(0.000001-90);
Alternatively, the polymer has a structure represented by Formula II,

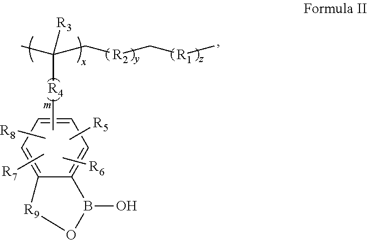

Wherein, R1 or R2 is selected from the following structures and salts thereof:

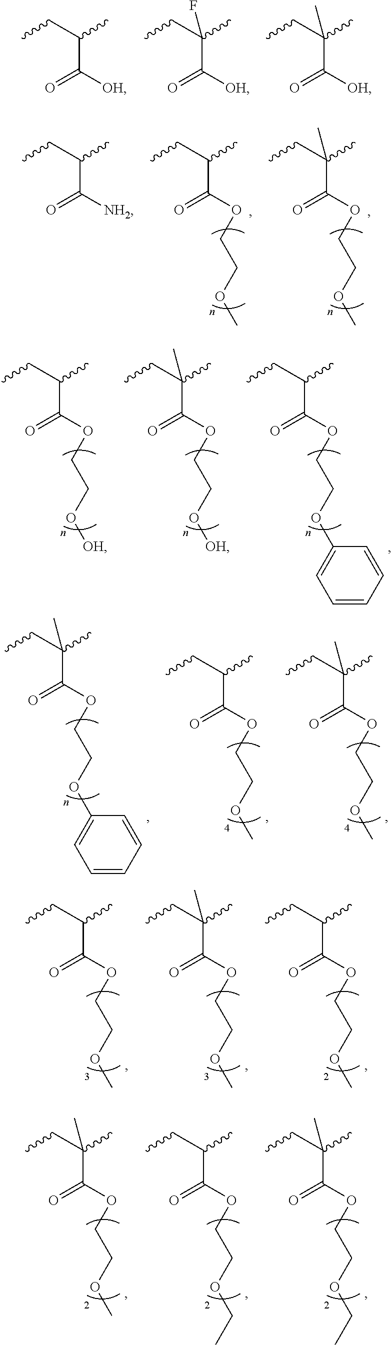

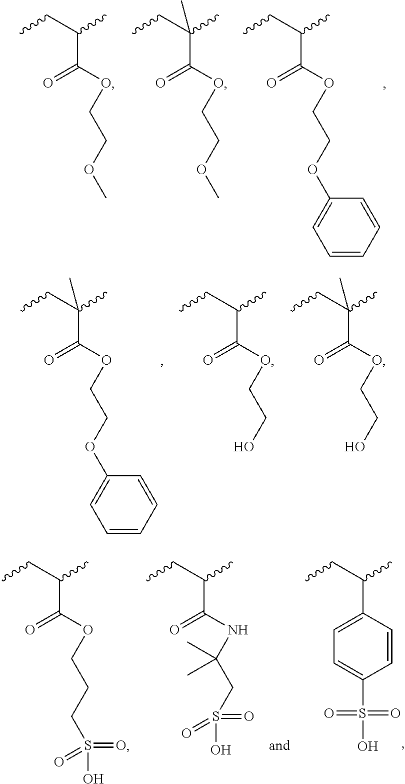

wherein n is an integer greater than or equal to 0;
R3 is selected from the following structures:

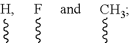

R4 is selected from the following structures:

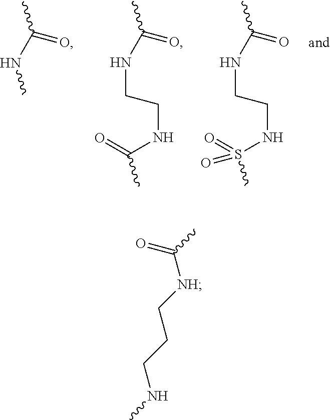

R5 or R6 or R7 or R8 is selected from the following structures:

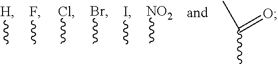

R9 is selected from the following structures:

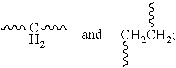

m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90);
Alternatively, the polymer has a structure represented by Formula III,

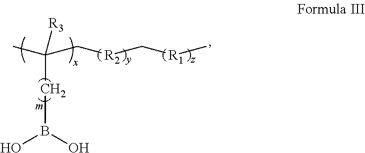

Wherein, R1 or R2 is selected from the following structures:

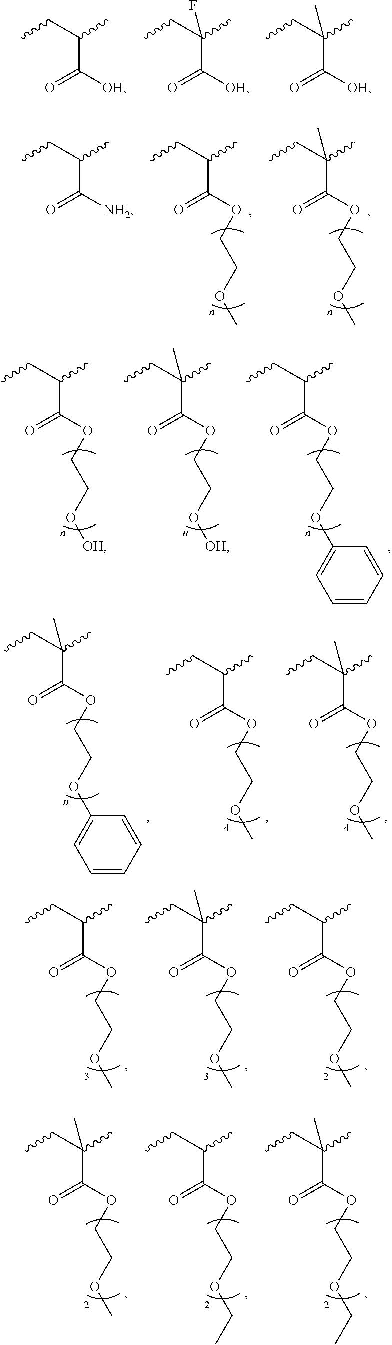

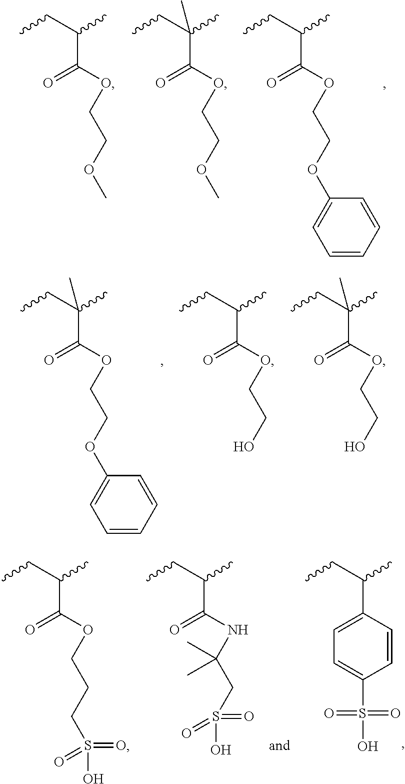

wherein n is an integer greater than or equal to 0;
R3 is selected from the following structures:

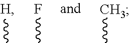

m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90).

In some embodiments, according to the pharmaceutical composition of the present application, wherein the polymer comprising at least one boronic acid group is selected from:

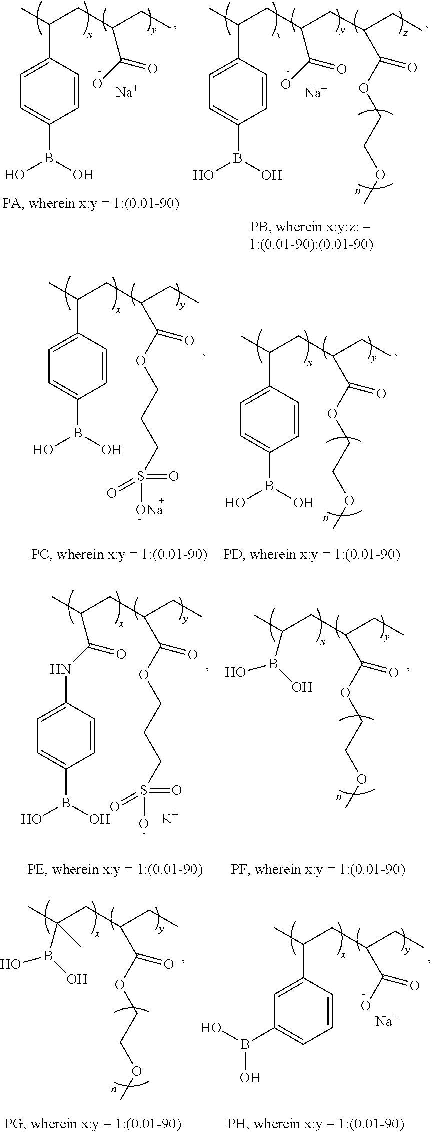

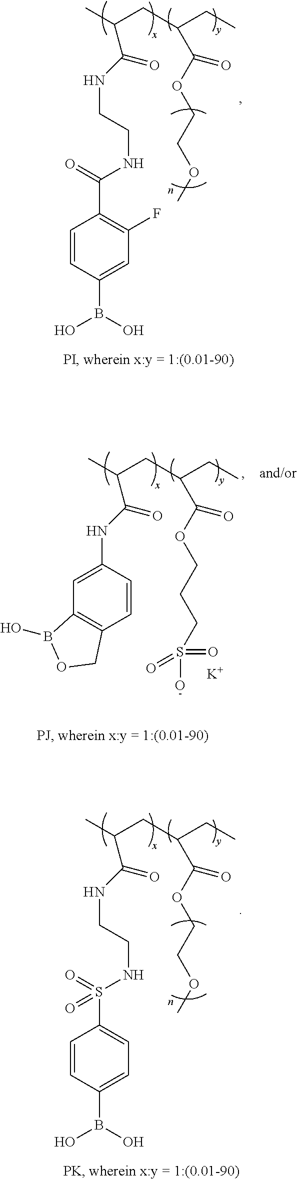

In some embodiments, according to the pharmaceutical composition of the present application, wherein the polymer is selected from the following group:

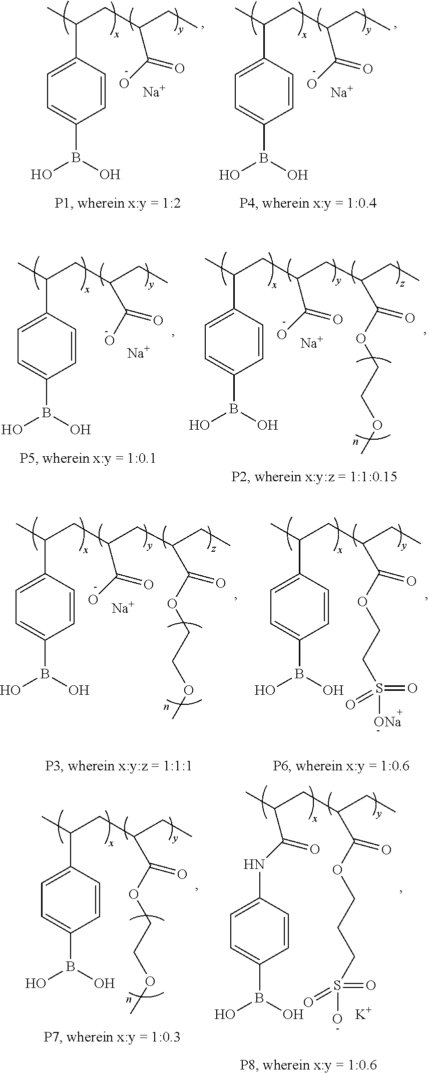

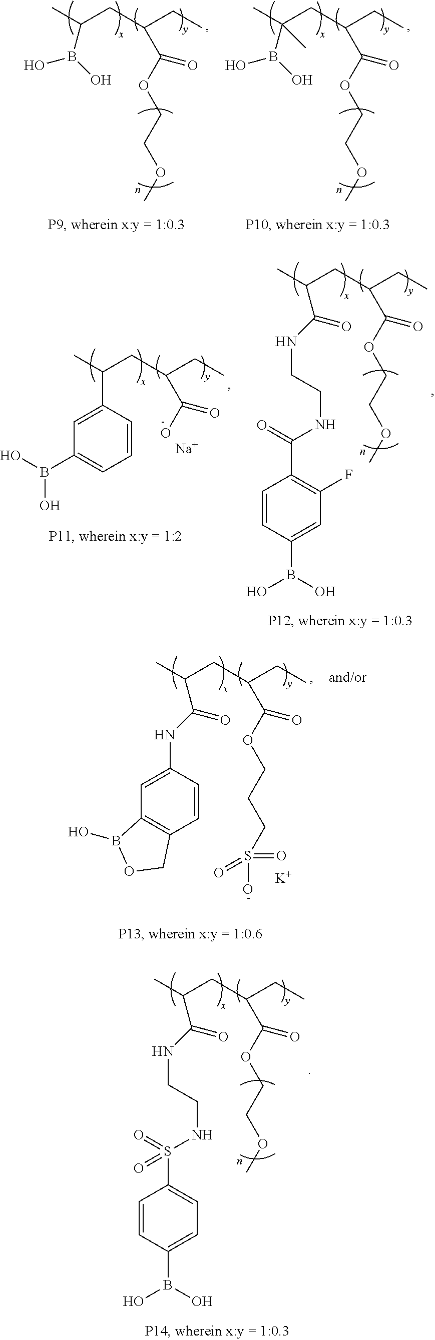

In some embodiments, according to the pharmaceutical composition of the present application, wherein the pharmaceutical activity comprises that, compared with a control group, the proportion of the saccharide that has been degraded enzymatically in the subject to whom the polymer comprising at least one boronic acid group is administered is reduced, and the control group is the subject to whom the polymer comprising at least one boronic acid group is not administered.

In some embodiments, according to the pharmaceutical composition of the present application, wherein the enzymatic hydrolysis comprises enzymatic hydrolysis by a related saccharidase, the related saccharidase comprises a glycosylase.

In some embodiments, according to the pharmaceutical composition of the present application, which is used to reduce the saccharide level.

In some embodiments, according to the pharmaceutical composition of the present application, which is used to prevent an increase in the saccharide level.

In some embodiments, according to the pharmaceutical composition of the present application, wherein the saccharide is selected from: monosaccharide, disaccharide, polysaccharide, and/or substance comprising the monosaccharide, the disaccharide and/or the polysaccharide.

In some embodiments, according to the pharmaceutical composition of the present application, which is used to treat or prevent saccharide-related diseases or disorders.

In some embodiments, according to the pharmaceutical composition of the present application, the saccharide-related diseases or disorders are selected from: obesity, diabetes and/or fatty liver.

In some embodiments, according to the pharmaceutical composition of the present application, which does not comprise other hypoglycemic drugs as the pharmaceutically active ingredient.

In some embodiments, according to the pharmaceutical composition of the present application, wherein the other hypoglycemic drugs are selected from insulin and the analogue thereof, insulin secretagogue, metformin drug, α-glucosidase inhibitor, insulin sensitizer, peroxisome proliferator-activated receptor agonist (PPAR agonist), GPR40 agonist, JNK inhibitor, pan-AMPK activator, incretin analogue, glucokinase agonist (GKA), G protein-coupled receptor agonist (GPCR agonists), SGLT1 inhibitor, SGLT2 inhibitor, DPP-4 inhibitor, glucagon receptor agonist (GCGR agonist), GIP receptor agonist, GSK-3 inhibitor, amylin analogue, vanadium-containing compound, GFAT inhibitor, 11β-HSD1 inhibitor, Sirtuin-1 (SIRT-1) agonist, PTP1B inhibitor, PI3K agonist, GLP-2 receptor agonist, and/or GLP-1 receptor agonist.

In some embodiments, according to the pharmaceutical composition of the present application, which is formulated as a preparation for oral administration.

Those skilled in the art can easily perceive other aspects and advantages of the present application from the detailed description below. In the following detailed description, only exemplary embodiments of the present application are shown and described. As those skilled in the art will recognize, the content of this application enables those skilled in the art to make changes to the disclosed specific embodiments without departing from the spirit and scope of the invention involved in this application. Correspondingly, the drawings and descriptions in the specification of the present application are merely exemplary, rather than restrictive.

### BRIEF DESCRIPTION OF THE DRAWINGS

The specific features of the invention involved in this application are shown in the appended claims. The characteristics and advantages of the invention involved in this application can be better understood by referring to the exemplary embodiments and the accompanying drawings described in detail below. A brief description of the drawings is as follows:

FIGS. 1-12 show the chemical reaction process for preparing the polymer comprising at least one boronic acid group of the present application and the polymer of the comparative example;

FIG. 13 shows the concentration of glucose in the solution outside the dialysis bag;

FIG. 14 shows the area under the curve (AUC) after plotting the concentration of glucose outside the dialysis bag against time;

FIG. 15 shows the body weight changes of mice;

FIG. 16 shows the distribution of the polymer of the present application in mice in vivo;

FIG. 17 shows the blood glucose concentration in the oral glucose tolerance test (OGTT) and the intraperitoneal glucose tolerance test (IPGTT);

FIG. 18 shows the area under the curve (AUC) in the oral glucose tolerance test (OGTT) and the intraperitoneal glucose tolerance test (IPGTT);

FIGS. 19-20 show the blood glucose concentration and the area under the curve (AUC) of mice after oral administration of glucose, respectively;

FIGS. 21-22 show the blood glucose concentration and the area under the curve (AUC) of mice after oral administration of maltose, respectively;

FIGS. 23-24 show the blood glucose concentration and the area under the curve (AUC) of mice after oral administration of sucrose, respectively;

FIGS. 25-26 show the blood glucose concentration and the area under the curve (AUC) of mice after oral administration of dextrin, respectively;

FIGS. 27-29 show the blood glucose concentration of mice after oral administration of blueberry jam, Coca Cola, rice gruel, respectively;

FIG. 30 shows the elevated blood glucose values of mice after oral administration of real food;

FIGS. 31-34 show the blood glucose concentration of mice with diet-induced obesity after oral administration of glucose, sucrose, maltose and dextrin, respectively;

FIGS. 35-38 show the area under the curve (AUC) of mice with diet-induced obesity after oral administration of glucose, sucrose, maltose and dextrin, respectively;

FIGS. 39-41 show the blood glucose concentration of mice with diet-induced obesity after oral administration of blueberry jam, Coca Cola, and rice gruel, respectively;

FIG. 42 shows the elevated blood glucose values of mice with diet-induced obesity after oral administration of real food;

FIGS. 43-44 show the blood glucose concentration and the area under the curve (AUC) of Streptozotocin (STZ)—induced mice after oral administration of glucose, respectively;

FIG. 45 shows the relative contents of total cholesterol, triglycerides and free fatty acids in the livers of the three groups of mice;

FIG. 46 shows the optical microphotographs of liver sections of the three groups of mice after being stained with Oil Red 0.

### DETAILED DESCRIPTION

The following specific examples illustrate the implementation of the invention of the present application. Those familiar with this technology can easily understand other advantages and effects of the invention of this application from the content disclosed in this specification.

In the present application, the term “saccharide-related diseases or disorders” generally refers to diseases or disorders caused by the effects of saccharides on the body. For example, in the present application, the saccharide-related diseases or disorders may be diabetes, fatty liver, or obesity. For another example, in the present application, the saccharide-related diseases or disorders may be type I diabetes or type II diabetes.

In the present application, the term “polymer comprising at least one boronic acid group” generally refers to a class of polymer comprising one or more boronic acid groups. For example, the polymer comprising at least one boronic acid group described in the present application may have a structure shown in any one of Formula I, Formula II, and Formula III described below. For another example, the polymer comprising at least one boronic acid group described in the present application may have a structure shown in any one of PA, PB, PC, PD, PE, PF, PG, PH, PI, PJ, and PK as described below. For another example, the polymer comprising at least one boronic acid group described in the present application may also have a structure shown in any one of P1, P2, P3, P4, P5, P6, P7, P8, P9, P10, P11, P12, P13 and P14.

In the present application, the term “therapeutically or prophylactically effective amount” generally refers to a dose that is effective for treatment or prevention. The specific dosage level will depend on a number of pharmacokinetic factors, including the activity of the used polymer comprising at least one boronic acid group as described in the present application, the route of administration, the time of administration, the rate of excretion of the polymer of the present application, the duration of treatment, other drugs used in combination with the polymer of the present application, the age, gender, weight, condition, general health and previous medical history of the treated patient, as well as similar factors known in the medical field. A physician or veterinarian with ordinary skills in the art can easily determine and prescribe the desired effective amount of the polymer of the present application.

In the present application, the term “saccharide level” generally refers to the content of polyhydroxy aldehydes or polyhydroxy ketones and their condensation polymers and certain derivatives. For example, in the present application, the saccharide level may be the content of monosaccharides, disaccharides or polysaccharides. For another example, in the present application, the saccharide level may be the concentration of glucose in the blood.

In the present application, the term “pharmaceutically active ingredient” generally refers to a class of substances that have a pharmacological activity in the prevention, diagnosis, symptomatic relief or treatment of diseases or that can affect the functions or structure of the body. For example, in the present application, the pharmaceutically active ingredient may be the polymer comprising at least one boronic acid group described in the present application, and after the subject has been administered the polymer comprising at least one boronic acid group, compared with the control group, the proportion of the saccharide that has been degraded enzymatically is reduced, and the control group is the subject to whom the polymer comprising at least one boronic acid group is not administered.

In the present application, the term “glycosylases”, of which the enzymatic code is EC 3.2, generally refers to a class of hydrolases (enzymatic code is EC 3), which plays an important role in the process of hydrolysis and synthesis of biosugar and glycoconjugates. For example, in the present application, the glycosylase may comprise glycosidase (enzymatic number EC 3.2.1). For another example, in the present application, the glycosylase may comprise at least one of α-glucosidase, α-amylase, pullulanase, debranching enzyme, maltase, sucrase, lactase, fungal glucanase, β-amylase and glucoamylase.

In the present application, the term “direct interaction” generally refers to a corresponding effect through a direct interaction between substances. For example, in the present application, the polymer comprising at least one boronic acid group can directly interact with the saccharide. For example, the polymer comprising at least one boronic acid group can directly interact with the saccharide through covalent bonds or intermolecular interactions, thereby preventing the saccharide bound to the polymer comprising at least one boronic acid group from being degraded enzymatically, thus preventing the saccharide from being absorbed by the body. For another example, the polymer comprising at least one boronic acid group can be directly bound with the nucleophilic group contained in the saccharide, such as a hydroxyl group, or an amino group, or a sulfhydryl group, or a carboxyl group through a covalent chemical reaction, thereby preventing the saccharide reacted with the polymer comprising at least one boronic acid group from being degraded enzymatically, thus preventing the saccharide from being absorbed by the body. For another example, the polymer comprising at least one boronic acid group can be directly bound by a covalent chemical reaction between the boronic acid group and the nucleophilic group on the surface of the saccharide aggregate, such as a hydroxyl group or an amino group or a sulfhydryl group or a carboxyl group, thereby preventing the saccharide aggregate from being degraded enzymatically, thus preventing the saccharide from being absorbed by the body, thus reducing the proportion of the saccharide that has been degraded enzymatically in the subject, thus reducing the saccharide level in the subject, or preventing an increase of the saccharide level in the subject, or preventing or slowing down the absorption of the saccharide by the subject, or treating or preventing saccharide-related diseases or disorders. In addition, non-“direct interaction” usually refers to the fact that there is no direct interaction between substances to exert the corresponding effect. For example, the boronic acid group is only used as a component of the hypoglycemic drug carrier, and only plays the roles of loading, transporting and releasing the hypoglycemic drug. In the absence of the hypoglycemic drug, the boronic acid group cannot directly reduce the saccharide level in the subject, then the interaction between the boronic acid group-containing substance and the saccharide does not belong to the “direct interaction” as described in the present application. For another example, the boronic acid group is only used as a component of the insulin carrier, and the release of insulin is triggered through the interaction between the boronic acid group and the saccharide, which in turn allows the insulin to exert a hypoglycemic effect. However, the boronic acid group does not directly exert a hypoglycemic effect, so, the interaction between the boronic acid group-containing substance and the saccharide does not belong to the “direct interaction” as described in the present application.

In the present application, the term “other hypoglycemic drugs” generally refers to drugs that can reduce the saccharide level. For example, in the present application, the other hypoglycemic drugs may be selected from insulin and the analogue thereof, insulin secretagogues, metformin drugs, α-glucosidase inhibitors, insulin sensitizers, peroxisome proliferator-activated receptor agonists (PPAR agonists), GPR40 agonists, JNK inhibitors, pan-AMPK activators, incretin analogues, glucokinase agonists (GKA), G protein-coupled receptor agonists (GPCR agonists), SGLT1 inhibitors, SGLT2 inhibitors, DPP-4 inhibitors, glucagon receptor agonists (GCGR agonists), GIP receptor agonists, GSK-3 inhibitors, amylin analogues, vanadium-containing compounds, GFAT inhibitors, 11β-HSD1 inhibitors, Sirtuin-1 (SIRT-1) agonists, PTP1B inhibitors, PI3K agonists, GLP-2 receptor agonists, and/or GLP-1 receptor agonists. Among them, the α-glucosidase inhibitors may comprise acarbose, which is a marketed hypoglycemic drug that inhibits the activity of α-glucosidase to reduce the digestion and absorption of polysaccharides and disaccharides such as starch and maltose by human body, thereby reducing the blood glucose after a meal, but it has no effect on monosaccharide.

In the present application, the term “diabetes” generally refers to a group of metabolic diseases characterized by hyperglycemia. For example, in the present application, the diabetes may be type I diabetes. For another example, in the present application, the diabetes may be type II diabetes.

In the present application, the term “comprising” generally refers to the inclusion of explicitly specified features, but not excluding other elements.

In the present application, the term “about” generally refers to varying within a range of 0.5%-10% above or below the specified value, for example, varying within a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below the specified value.

In one aspect, the present application provides a method for treating or preventing saccharide-related diseases or disorders, comprising administering a therapeutically or prophylactically effective amount of a polymer comprising at least one boronic acid group to a subject having or at risk of having the saccharide-related diseases or disorders. Wherein, the saccharide-related diseases or disorders may be selected from: obesity, diabetes and/or fatty liver. Wherein, diabetes may be type I diabetes or type II diabetes.

In another aspect, the present application provides a method for reducing the saccharide level of a subject, comprising administering a therapeutically or prophylactically effective amount of a polymer comprising at least one boronic acid group to the subject.

In yet another aspect, the present application provides a method for preventing an increase in the saccharide level of a subject, comprising administering a therapeutically or prophylactically effective amount of a polymer comprising at least one boronic acid group to the subject.

In the fourth aspect, the present application provides a method for preventing or slowing down the absorption of saccharide by a subject, comprising administering a therapeutically or prophylactically effective amount of a polymer comprising at least one boronic acid group to the subject.

Saccharides and Saccharide Levels

In the present application, the saccharides may be selected from: monosaccharides, disaccharides, polysaccharides, and/or substances comprising the monosaccharides, the disaccharides and/or the polysaccharides. For example, the saccharides may be glucose or fructose, may also be maltose, starch or glycogen, and may also be sucrose or dextrin. For another example, the saccharides may be food comprising monosaccharides, disaccharides and/or polysaccharides, e.g., fruits, jam, beverages, gruel or rice.

In the present application, the saccharide level may be the saccharide level of the subject after a meal. For example, the saccharide level may be the saccharide level after breakfast, may also be the saccharide level after lunch, after dinner or after supper, and may also be the saccharide level after eating snacks. For example, the saccharide level may be the saccharide level 2 hours after a meal.

In the present application, the saccharide level may be the blood glucose concentration, which may usually be detected by a blood glucose meter. The blood glucose concentration of normal people may be usually: 3.9-5.6 mmol/1 on an empty stomach, and less than 7.8 mmol/12 hours after a meal. When the fasting blood glucose exceeds (including) 7.0 mmol/1 or/and the blood glucose 2 hours after a meal exceeds (including) 11.1 mmol/1, or the blood glucose at any time exceeds (including) 11.1 mmol/1, the subject may be considered as a person with hyperglycemia or a person with a higher saccharide level.

In the present application, the saccharide level may also be the concentration of glycosylated hemoglobin (HbA1c) in the blood. Glycosylated hemoglobin is the product of the combination of hemoglobin in red blood cells and saccharides in serum, which may usually be detected by a glycosylated hemoglobin analyzer. The ratio of glycosylated hemoglobin in normal people may be 4%-6%. If the ratio is higher than this range, it may be considered that the saccharide level in the body is relatively high.

In the present application, the saccharide level may also be the concentration of glycosylated serum protein (GSP) in the blood. Glycosylated serum protein is the product of a non-enzymatic saccharification reaction between the glucose in the blood and the N-terminal amino group of albumin and other protein molecules, which may usually be detected by a nitroblue tetrazolium colorimetric method (NBT method) or a ketoamine oxidase method. The concentration range of glycosylated serum protein in normal people may: NBT method: <285 μmol/L, ketoamine oxidase method: 122-236 μmol/L. If higher than this range, it may be considered that the saccharide level in the body is relatively high.

In the present application, the saccharide level may also be the urine glucose concentration. The so-called urine glucose concentration generally refers to the glucose concentration in the urine, which may be usually be detected by Benedict urine glucose qualitative examination method or a urine glucose test paper method. Normal people have very little urine glucose, or there should be no glucose in the urine, so the urine glucose test of normal people should be negative. If the urine glucose test is positive, it may be be considered that the body contains higher saccharide, or the saccharide level in the body is relatively high.

A Polymer Comprising at Least One Boronic Acid Group

In the present application, the polymer comprising at least one boronic acid group may have a structure represented by Formula I,

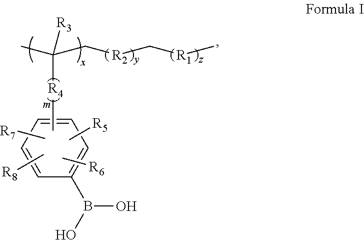

Wherein, R1 or R2 is selected from the following structures and salts thereof:

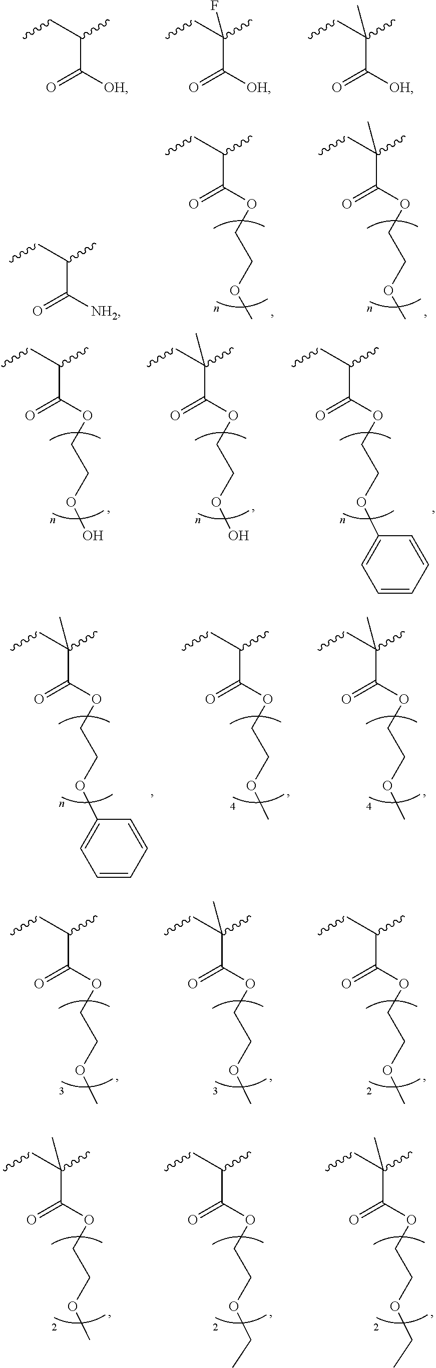

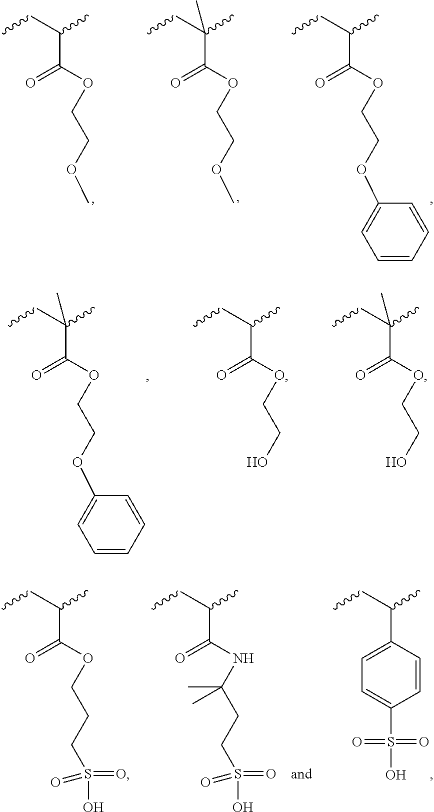

wherein n is an integer greater than or equal to 0;
R3 is selected from the following structures:

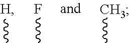

R4 is selected from the following structures:

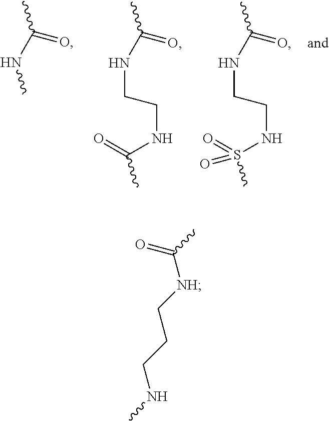

R5 or R6 or R7 or R8 is selected from the following structures:

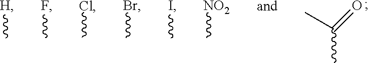

m is an integer greater than or equal to 0, x is a positive integer greater than or equal to 1, y and z are integers greater than or equal to 0, and when y is not equal to 0, x:y=1:(0.000001-90); when z is not equal to 0, x:z=1:(0.000001-90);
Alternatively, the polymer may have a structure represented by Formula II,

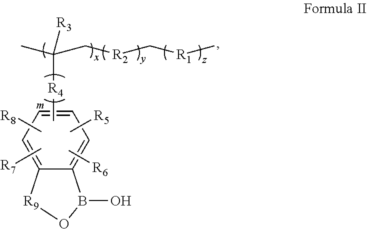

Wherein, R1 or R2 is selected from the following structures and salts thereof:

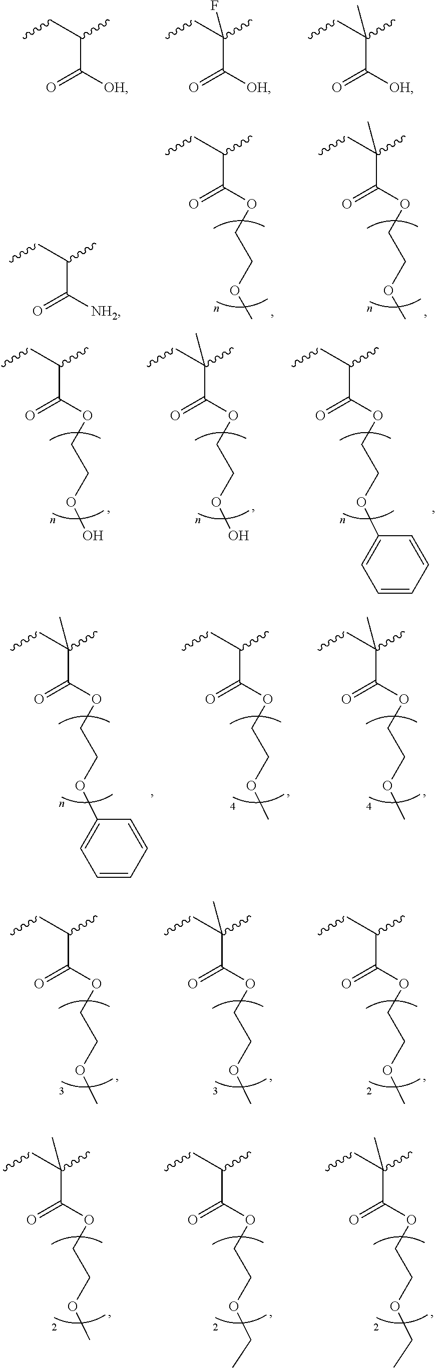

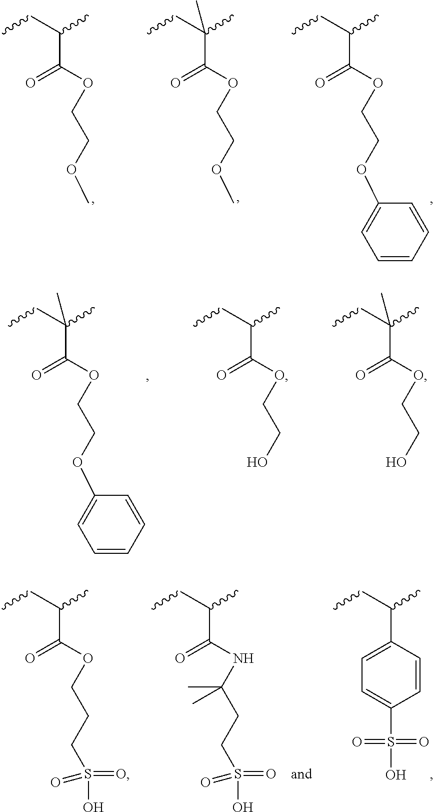

wherein n is an integer greater than or equal to 0;
R3 is selected from the following structures:

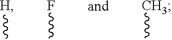

R4 is selected from the following structures:

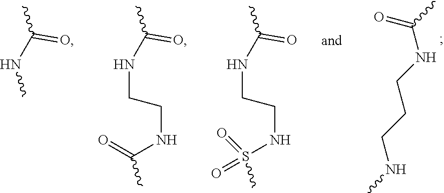

R5 or R6 or R7 or R8 is selected from the following structures:

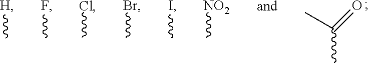

R9 is selected from the following structures:

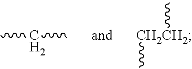

m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90);
Alternatively, the polymer may have a structure represented by Formula III,

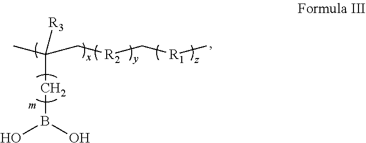

Wherein, R1 or R2 is selected from the following structures:

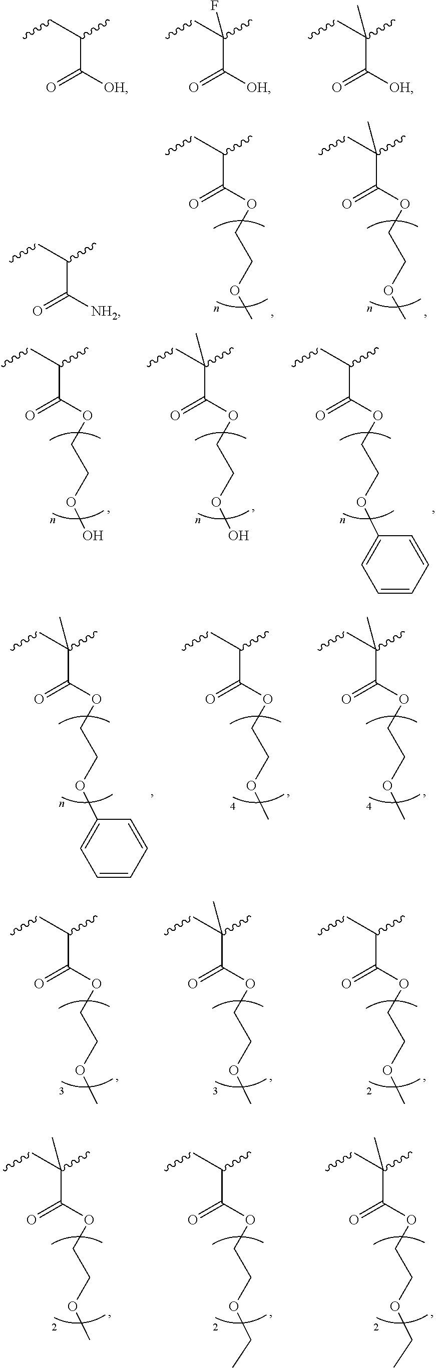

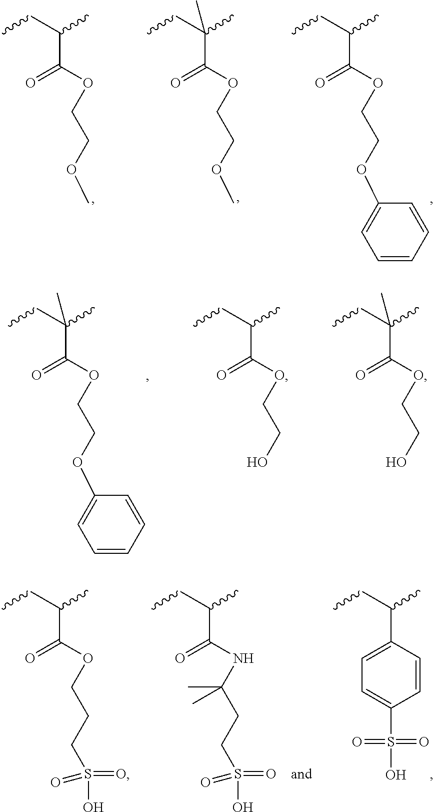

wherein n is an integer greater than or equal to 0;
R3 is selected from the following structures:

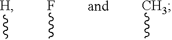

m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90).

In the present application, the polymer comprising at least one boronic acid group may be selected from:

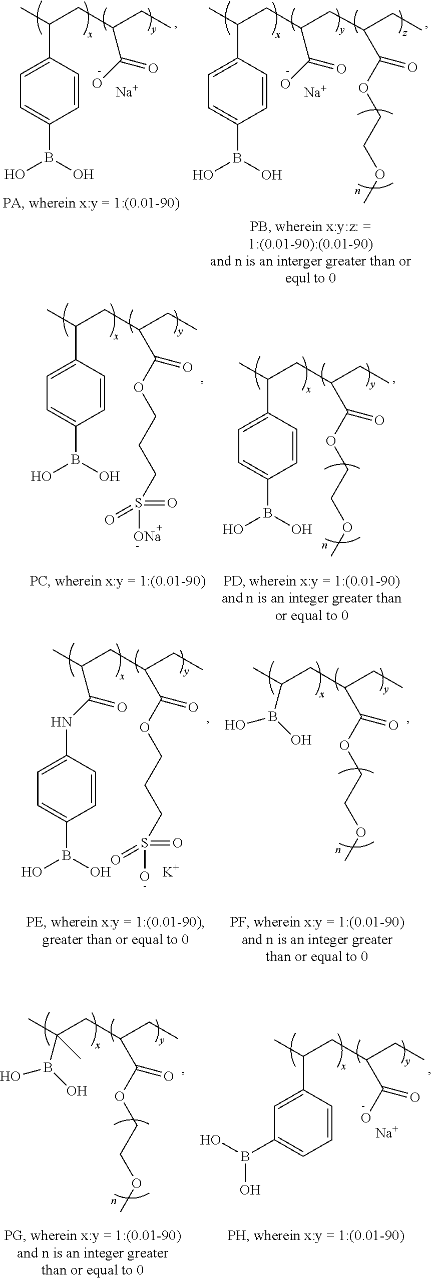

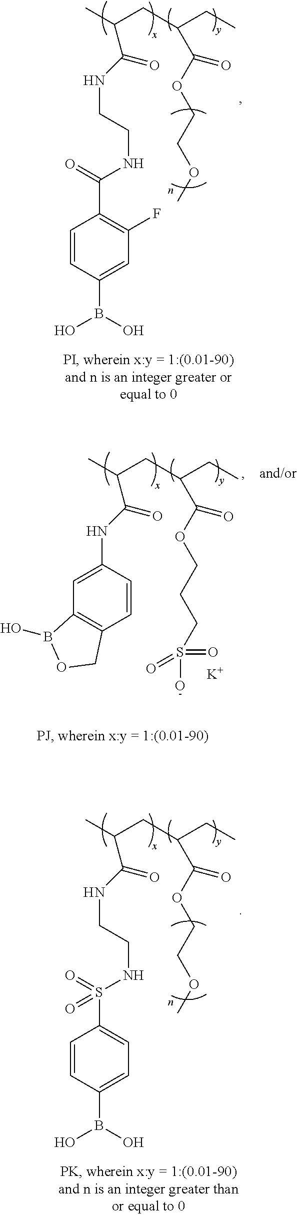

In the present application, the polymer comprising at least one boronic acid group may be selected from:

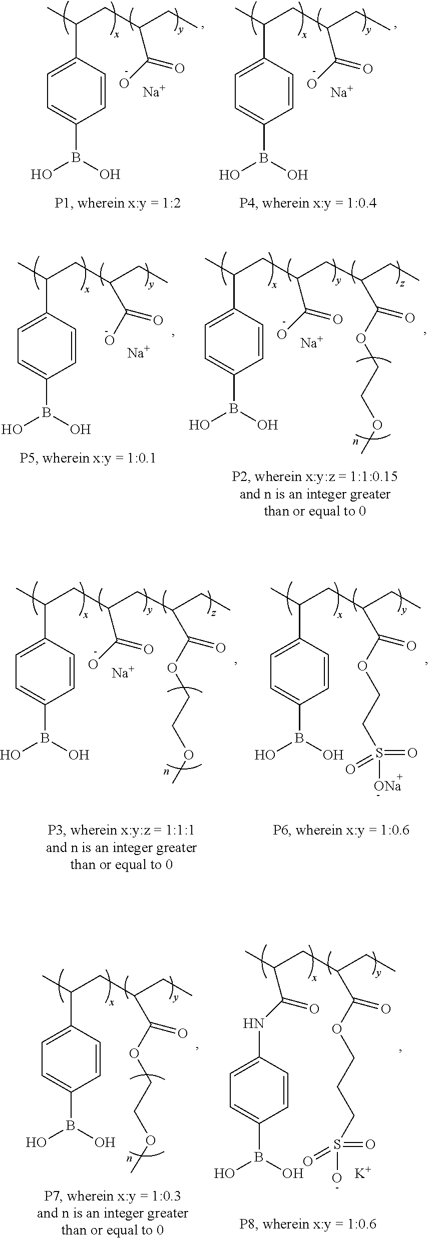

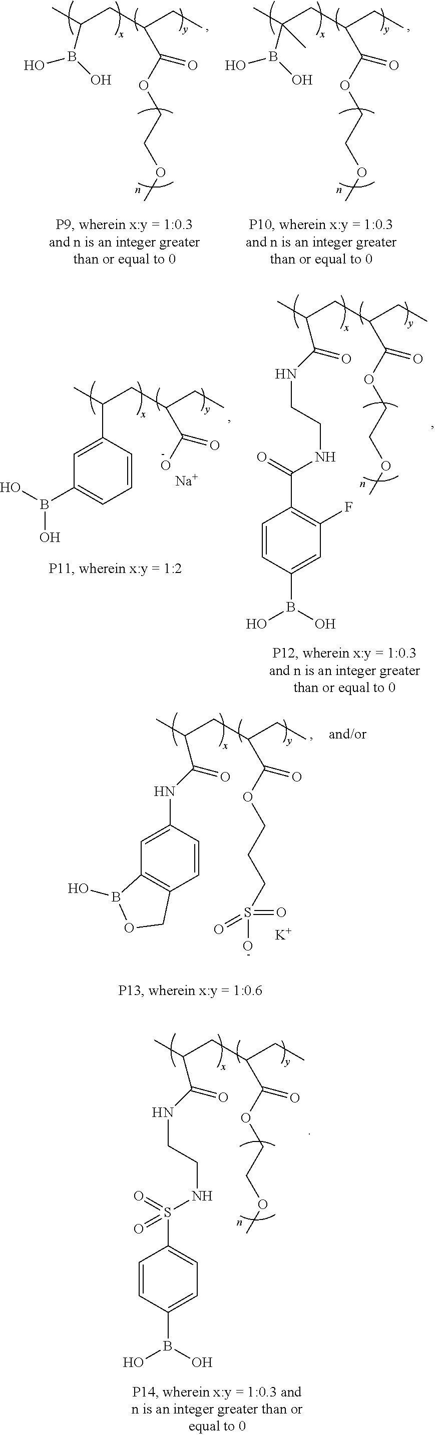

In the present application, the polymer comprising at least one boronic acid group may be formulated as an oral preparation, so that the subject may orally take the polymer comprising at least one boronic acid group described in the present application.

The Proportion of the Saccharide that has been Degraded Enzymatically is Reduced

In the present application, the polymer comprising at least one boronic acid group may be administered as a pharmaceutically active ingredient. Wherein, the pharmaceutical activity may comprise, compared with a control group, the proportion of the saccharide that has been degraded enzymatically in the subject to whom the polymer comprising at least one boronic acid group is administered is reduced, and the control group is the subject to whom the polymer comprising at least one boronic acid group is not administered. For example, in some embodiments, subjects in the experimental group are administered the polymer comprising at least one boronic acid group described in the present application, while those in the control group are not administered the polymer comprising at least one boronic acid group described in the present application, the subjects in the experimental group and the control group ingest the same type and content of saccharide (such as maltose, sucrose, starch, dextrin, glycogen), and the blood glucose concentration of the subjects is detected after a period of time (e.g., 2 hours after the ingestion of saccharide). The detected blood glucose concentration may be divided by the theoretical glucose concentration to get the saccharide digestibility. The lower the saccharide digestibility, the lower the proportion of saccharide that has been degraded enzymatically. The experimental results show that the saccharide digestibility of the experimental group is significantly lower than that of the control group, indicating that the saccharides are more bound by the polymer of the present application and less degraded by enzymes. In other words, compared with subjects to whom the polymer of the present application is not administered, the proportion of the saccharide that has been degraded enzymatically in the subject to whom the polymer of the present application is administered is reduced, thus indicating that the polymer of the present application has a strong ability to inhibit the degradation of saccharides by enzymes, and has the effect of reducing the digestion and absorption of saccharides by the body.

It should be noted that the theoretical glucose concentration may be calculated by the following method:
The theoretical glucose concentration of maltose=Initial maltose concentration;
The theoretical glucose concentration of sucrose=Initial sucrose concentration÷2;
The theoretical glucose concentration of starch=Initial starch concentration;
The theoretical glucose concentration of dextrin=Initial dextrin concentration;
The theoretical glucose concentration of glycogen=Initial glycogen concentration.

In addition, it should also be noted that, in some embodiments, saccharides may not need to be enzymatically degraded before being absorbed by the human body, such as monosaccharides, so when the polymer comprising at least one boronic acid group described in the present application is administered as a pharmaceutically active ingredient, the polymer interacts with the saccharide directly, thereby reducing the digestion and absorption of the saccharides by the body. During this process, since no enzymatic hydrolysis process is involved, the pharmaceutical activity may not comprise, compared with a control group, the proportion of the saccharide that has been degraded enzymatically in the subject to whom the polymer comprising at least one boronic acid group is administered is reduced, and the control group is the subject to whom the polymer comprising at least one boronic acid group is not administered.

In the present application, the enzymatic hydrolysis may comprise enzymatic hydrolysis by a related saccharidase, and the related saccharidase may comprise glycosylase. Wherein, the glycosylase may comprise glycosidase. In addition, the glycosylase may also comprise at least one of α-glucosidase, α-amylase, pullulanase, debranching enzyme, maltase, sucrase, lactase, fungal glucanase, β-amylase and glucoamylase.

Direct Interaction with Saccharides

In the present application, the polymer comprising at least one boronic acid group may interact with the saccharide directly. For example, the polymer comprising at least one boronic acid group may directly interact with the saccharide through covalent bonds or intermolecular interactions, thereby preventing the saccharide bound to the polymer comprising at least one boronic acid group from being degraded enzymatically, thus preventing the saccharide from being absorbed by the body. For another example, the polymer comprising at least one boronic acid group can be directly bound by a covalent chemical reaction between the boronic acid group and the nucleophilic group contained in the saccharide, such as a hydroxyl group, or an amino group, or a sulfhydryl group, or a carboxyl group, thereby preventing the saccharide reacted with the polymer comprising at least one boronic acid group from being degraded enzymatically, thus preventing the saccharide from being absorbed by the body. For another example, the polymer comprising at least one boronic acid group can be directly bound by a covalent chemical reaction between the boronic acid group and the nucleophilic group on the surface of the saccharide aggregate, such as a hydroxyl group or an amino group or a sulfhydryl group or a carboxyl group, thereby preventing the saccharide aggregate from being degraded enzymatically, thus preventing the saccharide from being absorbed by the body, thus reducing the proportion of the saccharide that has been degraded enzymatically in the subject, thus reducing the saccharide level in the subject, or preventing an increase of the saccharide level in the subject, or preventing or slowing down the absorption of the saccharide by the subject, or treating or preventing saccharide-related diseases or disorders.

In addition, it should be noted that, although some substances also have the effect of reducing the saccharide level or preventing an increase of the saccharide level or preventing or slowing down the absorption of the saccharide by the body, they do not belong to the direct interaction with saccharides as described in the present application. For example, a certain substance is only used as a carrier of hypoglycemic drugs and does not interact with the saccharide during the process of lowering the blood glucose, then this substance does not belong to the direct interaction with saccharides as described in the present application. For another example, a certain substance triggers or releases a certain hypoglycemic drug by interacting with the saccharide, thereby exerting a hypoglycemic effect through using this hypoglycemic drug, then this substance does not belong to the direct interaction with saccharides as described in the present application, either. For another example, a certain substance mainly stimulates pancreatic β cells to produce and release insulin, thereby reducing the blood glucose concentration with the insulin, then this substance does not belong to the direct interaction with saccharides as described in the present application, either. For another example, a certain substance prevents the saccharide from being degraded enzymatically mainly by reducing the activity of an enzyme (e.g., glucosidase) that hydrolyzes the saccharide or inactivating the enzyme, thereby delaying the absorption of saccharide by the body, thus reducing the postprandial blood glucose, then this substance does not belong to the direct interaction with saccharides as described in the present application, either. For another example, a certain substance reduces the glucose concentration in the blood mainly by increasing the ingestion and utilization of glucose by peripheral tissues (such as muscle and fat), then this substance does not belong to the direct interaction with saccharides as described in the present application, either. For another example, a certain substance reduces the insulin resistance mainly by improving the sensitivity of cells to the action of insulin, thereby reducing the blood glucose concentration with the insulin, then this substance does not belong to the direct interaction with saccharides as described in the present application, either. For another example, the boronic acid group of a substance is only used as a component of the hypoglycemic drug carrier, and only plays the roles of loading, transporting and releasing the hypoglycemic drug, and in the absence of the hypoglycemic drug, the boronic acid group cannot directly reduce the saccharide level in the subject, so the interaction between the substance containing the boronic acid group and the saccharide does not belong to the “direct interaction” as described in the present application. For another example, the boronic acid group of a substance is only used as a component of the insulin carrier, and the release of insulin is triggered by the action between the boronic acid group and the saccharide, which in turn enables insulin to exert a hypoglycemic effect, while the boronic acid group does not directly exert a hypoglycemic effect, so the interaction between the substance containing the boronic acid group and the saccharide does not belong to the “direct interaction” as described in the present application, either.

Other Hypoglycemic Drugs

In the present application, other hypoglycemic drugs may be administered to the subject before, simultaneously or after the administration of the polymer comprising at least one boronic acid group. Among them, the other hypoglycemic drugs may be selected from insulin and the analogue thereof, insulin secretagogues, metformin drugs, α-glucosidase inhibitors, insulin sensitizers, peroxisome proliferator-activated receptor agonists (PPAR agonists), GPR40 agonists, JNK inhibitors, pan-AMPK activators, incretin analogues, glucokinase agonists (GKA), G protein-coupled receptor agonists (GPCR agonists), SGLT1 inhibitors, SGLT2 inhibitors, DPP-4 inhibitors, glucagon receptor agonists (GCGR agonists), GIP receptor agonists, GSK-3 inhibitors, amylin analogues, vanadium-containing compounds, GFAT inhibitors, 11β-HSD1 inhibitors, Sirtuin-1 (SIRT-1) agonists, PTP1B inhibitors, PI3K agonists, GLP-2 receptor agonists, and/or GLP-1 receptor agonists.

Administration

In the present application, the administration may be administering orally, for example, oral administration. In addition, the administration may also be injection, for example, intravenous injection or intramuscular injection. For another example, the administration may also be intracavitary injection, for example, intraperitoneal injection.

Use of a Polymer Comprising at Least One Boronic Acid Group in the Preparation of a Medicament

In the fifth aspect, the present application also provides a use of a polymer comprising at least one boronic acid group in the preparation of a medicament, the medicament is used to treat or prevent saccharide-related diseases or disorders. Wherein, the saccharide-related diseases or disorders may comprise obesity, diabetes and/or fatty liver. For example, diabetes may be type I diabetes or type II diabetes.

In the use of the present application, wherein the medicament may be used to reduce the saccharide level, may also be used to prevent an increase in the saccharide level, and may also be used to prevent or slow down the absorption of the saccharide. Wherein, the saccharide may comprise: monosaccharide, disaccharide, polysaccharide and substance comprising the monosaccharide, the disaccharide and/or the polysaccharide. For example, the saccharide may be glucose or fructose, may also be maltose, and may also be sucrose or dextrin. For another example, the saccharide may be food comprising monosaccharide, disaccharide and/or polysaccharide, e.g., fruits, jam, beverages, gruel or rice.

In the use of the present application, the medicament may comprise a therapeutically or prophylactically effective amount of the polymer comprising at least one boronic acid group as the therapeutically or prophylactically active ingredient of the medicament. The medicament may be formulated as a preparation suitable for oral administration, so that the subject can orally take the medicament described in the present application.

In the use of the present application, the polymer may have a structure as shown in any one of the above Formula I, Formula II and Formula III, may also have a structure as shown in any one of PA, PB, PC, PD, PE, PF, PG, PH, PI, PJ and PK above, and may also have a structure as shown in any one of P1, P2, P3, P4, P5, P6, P7, P8, P9, P10, P11, P12, P13 and P14 below. Therefore, its specific structure will not be repeated here.

Pharmaceutical Composition

In the sixth aspect, the present application also provides a pharmaceutical composition, the pharmaceutically active ingredient of which comprise a polymer comprising at least one boronic acid group.

In the pharmaceutical composition of the present application, the polymer may have a structure as shown in any one of the above Formula I, Formula II and Formula III, may also have a structure as shown in any one of PA, PB, PC, PD, PE, PF, PG, PH, PI, PJ and PK above, and may also have a structure as shown in any one of P1, P2, P3, P4, P5, P6, P7, P8, P9, P10, P11, P12, P13 and P14 below. Therefore, its specific structure will not be repeated here.

In the pharmaceutical composition of the present application, the pharmaceutical activity may comprise, compared with a control group, the proportion of the saccharide that has been degraded enzymatically in the subject to whom the polymer comprising at least one boronic acid group is administered is reduced, and the control group is the subject to whom the polymer comprising at least one boronic acid group is not administered. For example, as described above, in some embodiments, the proportion of the saccharide that has been degraded enzymatically can be determined by the saccharide digestibility. The lower the saccharide digestibility, the lower the proportion of the saccharide that has been degraded enzymatically. The specific experimental and calculating methods will not be repeated here.

In the pharmaceutical composition of the present application, the enzymatic hydrolysis may comprise enzymatic hydrolysis by a related saccharidase, and the related saccharidase may comprise glycosylase. Wherein, the glycosylase may comprise glycosidase. In addition, the glycosylase may also comprise at least one of α-glucosidase, α-amylase, pullulanase, debranching enzyme, maltase, sucrase, lactase, fungal glucanase, β-amylase and glucoamylase.

The pharmaceutical composition of the present application may be used to reduce the saccharide level, may also be used to prevent an increase of the saccharide level, and may also be used to treat or prevent saccharide-related diseases or disorders. Wherein, the saccharides may be selected from: monosaccharides, disaccharides, polysaccharides, and/or substances comprising the monosaccharides, the disaccharides and/or the polysaccharides. For example, the saccharide may be glucose or fructose, may also be maltose, and may also be sucrose or dextrin. For another example, the saccharide may be food comprising monosaccharides, disaccharides and/or polysaccharides, e.g., fruits, jam, beverages, gruel or rice. In addition, the saccharide-related diseases or disorders may be selected from: obesity, diabetes and/or fatty liver. For example, the diabetes may be type I diabetes or type II diabetes.

The pharmaceutical composition of the present application may not comprise other hypoglycemic drugs as the pharmaceutically active ingredient, wherein the other hypoglycemic drugs may be selected from insulin and the analogue thereof, insulin secretagogues, metformin drugs, α-glucosidase inhibitors, insulin sensitizers, peroxisome proliferator-activated receptor agonists (PPAR agonists), GPR40 agonists, JNK inhibitors, pan-AMPK activators, incretin analogues, glucokinase agonists (GKA), G protein-coupled receptor agonists (GPCR agonists), SGLT1 inhibitors, SGLT2 inhibitors, DPP-4 inhibitors, glucagon receptor agonists (GCGR agonists), GIP receptor agonists, GSK-3 inhibitors, amylin analogues, vanadium-containing compounds, GFAT inhibitors, 11β-HSD1 inhibitors, Sirtuin-1 (SIRT-1) agonists, PTP1B inhibitors, PI3K agonists, GLP-2 receptor agonists, and/or GLP-1 receptor agonists.

In addition, the pharmaceutical composition of the present application may be formulated as a preparation for oral administration, so that the subject can orally take the pharmaceutical composition of the present application.

Without intending to be limited by any theory, the following examples are only to illustrate the polymer comprising at least one boronic acid group, the use and the pharmaceutical composition of the present application, and are not intended to limit the scope of the present application.

### EXAMPLES

### Example 1. Synthesis of the Polymer P1 Comprising at Least One Boronic Acid Group Described in the Present Application

The polymer P1 comprising at least one boronic acid group described in the present application, i.e., poly-(4-vinylphenylboronic acid-r-acrylic acid)-1-2, the synthetic steps of which are as follows:

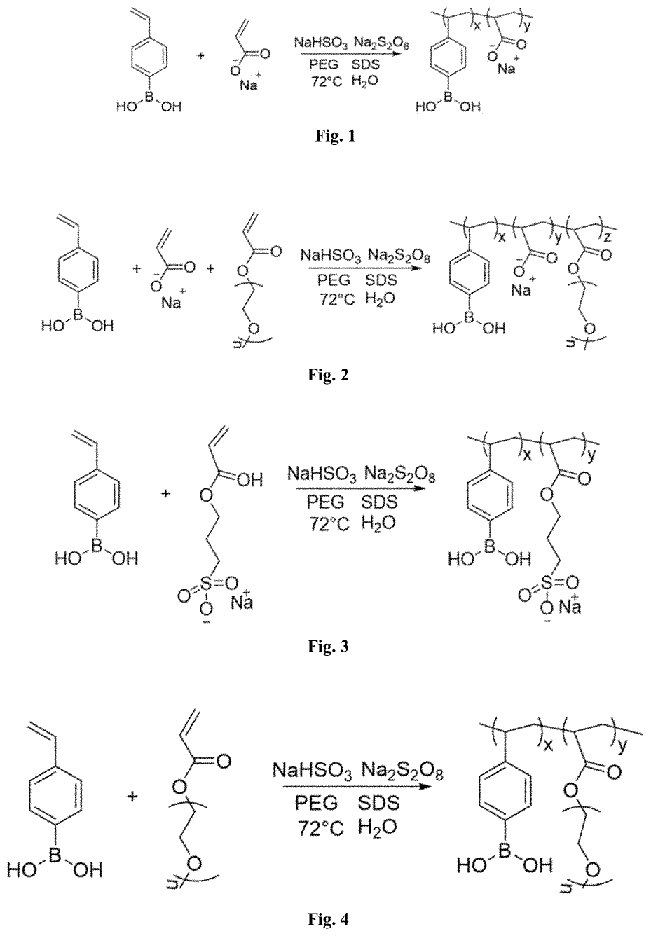

Into 40 mL of an aqueous solution comprising 4-vinylphenylboronic acid (1480.0 mg, 10.0 mmol), acrylic acid (1440.0 mg, 20.0 mmol), sodium bisulfate (94.7 mg, 0.9 mmol), polyethylene glycol (PEG, weight-average molecular weight 400, 15780.0 mg, 39.5 mmol), and sodium dodecyl sulfate (SDS, 3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Model: Sartorius Vivoflow 50, the corresponding molecular weight cut-off MWCO was 10000) driven by a peristaltic pump. After then, it was freeze-dried to obtain a white powder P1, the average yield of which was 91%. The chemical reaction process of the above synthetic steps is shown in FIG. 1, where x:y=1:2. The synthesized P1 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., the characteristic chemical shifts) at 7.52, 6.74, 2.19, 1.72, 1.56, indicating that P1 has the chemical structure of the reaction product in FIG. 1.

### Example 2. Synthesis of the Polymer P2 Comprising at Least One Boronic Acid Group Described in the Present Application

The polymer P2 comprising at least one boronic acid group described in the present application, i.e., poly-(4-vinylphenylboronic acid-r-acrylic acid-r-polyethylene glycol monoacrylate)-1-1-0.15, the synthetic steps of which are as follows:

Into 40 mL of an aqueous solution containing 4-vinylphenylboronic acid (1480.0 mg, 10.0 mmol), acrylic acid (720.0 mg, 10.0 mmol), polyethylene glycol monoacrylate (weight-average molecular weight 480, 720.0 mg, 1.5 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Model: Sartorius Vivoflow 50, the corresponding molecular weight cut-off MWCO was 10000) driven by a peristaltic pump. After then, it was freeze-dried to obtain a white powder P2, the average yield of which was 85%. The chemical reaction process of the above synthetic steps is shown in FIG. 2, where x:y:z=1:1:0.15 and n is an integer greater than or equal to 0. The synthesized P2 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., the characteristic chemical shifts) at 7.69, 6.78, 3.79-2.97, 2.17-1.00, indicating that P2 has the chemical structure of the reaction product in FIG. 2.

### Example 3. Synthesis of the Polymer P3 Comprising at Least One Boronic Acid Group Described in the Present Application

The polymer P3 comprising at least one boronic acid group described in the present application, i.e., poly-(4-vinylphenylboronic acid-r-acrylic acid-r-polyethylene glycol monoacrylate)-1-1-1, the synthetic steps of which are as follows:

Into 40 mL of an aqueous solution containing 4-vinylphenylboronic acid (1480.0 mg, 10.0 mmol), acrylic acid (720.0 mg, 10.0 mmol), polyethylene glycol monoacrylate (weight-average molecular weight 480, 4800.0 mg, 10.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white powder P3, the average yield of which was 87%. The chemical reaction process of the above synthetic steps is shown in FIG. 2, where x:y:z=1:1:1 and n is an integer greater than or equal to 0. The synthesized P3 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 7.72, 6.84, 3.88-3.59, 2.41-1.56, indicating that P3 has the chemical structure of the reaction product in FIG. 2.

### Example 4. Synthesis of the Polymer P4 Comprising at Least One Boronic Acid Group Described in the Present Application

The polymer P4 comprising at least one boronic acid group described in the present application, i.e., poly-(4-vinylphenylboronic acid-r-acrylic acid)-1-0.4, the synthetic steps of which are as follows:

Into 40 mL of an aqueous solution containing 4-vinylphenylboronic acid (1480.0 mg, 10.0 mmol), acrylic acid (288.0 mg, 4.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white powder P4, the average yield of which was 88%. The chemical reaction process of the above synthetic steps is shown in FIG. 1, where x:y=1:0.4. The synthesized P4 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 7.59, 6.74, 1.92-1.19, indicating that P4 has the chemical structure of the reaction product in FIG. 1.

### Example 5. Synthesis of the Polymer P5 Comprising at Least One Boronic Acid Group Described in the Present Application

The polymer P5 comprising at least one boronic acid group described in the present application, i.e., poly-(4-vinylphenylboronic acid-r-acrylic acid)-1-0.1, the synthetic steps of which are as follows:

Into 40 mL of an aqueous solution containing 4-vinylphenylboronic acid (1480.0 mg, 10.0 mmol), acrylic acid (72.0 mg, 1.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white powder P5, the average yield of which was 86%. The chemical reaction process of the above synthetic steps is shown in FIG. 1, where x:y=1:0.1. The synthesized P5 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 7.52, 6.74, 2.19-1.15, indicating that P5 has the chemical structure of the reaction product in FIG. 1.

### Example 6. Synthesis of the Polymer P6 Comprising at Least One Boronic Acid Group Described in the Present Application

The polymer P6 comprising at least one boronic acid group described in the present application, i.e., poly-(4-vinylphenylboronic acid-r-(3-sulfopropyl acrylate potassium salt))-1-0.6, the synthetic steps of which are as follows:

Into 40 mL of an aqueous solution containing 4-vinylphenylboronic acid (1480.0 mg, 10.0 mmol), 3-sulfopropyl acrylate potassium salt (1392.0 mg, 6.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white powder P6, the average yield of which was 89%. The chemical reaction process of the above synthetic steps is shown in FIG. 3, where x:y=1:0.6. The synthesized P6 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 7.56, 6.89, 2.86, 2.63-0.85, indicating that P6 has the chemical structure of the reaction product in FIG. 3.

### Example 7. Synthesis of the Polymer P7 Comprising at Least One Boronic Acid Group Described in the Present Application

The polymer P7 comprising at least one boronic acid group described in the present application, i.e., poly-(4-vinylphenylboronic acid-r-polyethylene glycol monoacrylate)-1-0.3, the synthetic steps of which are as follows:

Into 40 mL of an aqueous solution containing 4-vinylphenylboronic acid (1480.0 mg, 10.0 mmol), polyethylene glycol monoacrylate (weight-average molecular weight 480, 1440.0 mg, 3.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white solid P7, the average yield of which was 90%. The chemical reaction process of the above synthetic steps is shown in FIG. 4, where x:y=1:0.3 and n is an integer greater than or equal to 0. The synthesized P7 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 7.66, 6.77, 4.12-2.77, 2.15-1.05, indicating that P7 has the chemical structure of the reaction product in FIG. 4.

### Example 8. Synthesis of the Polymer P8 Comprising at Least One Boronic Acid Group Described in the Present Application

The polymer P8 comprising at least one boronic acid group described in the present application, i.e., poly-(4-acrylamidophenylboronic acid-r-(3-sulfopropyl acrylate potassium salt))-1-0.6, the synthetic steps of which are as follows:

Into a tetrahydrofuran (THF) solution containing 4-aminophenylboronic acid (1.37 g, 10 mmol) was added 10 ml triethylamine (Et₃N), then added dropwise a solution of acryloyl chloride (0.95 g, 10 mmol) in tetrahydrofuran slowly, stirred at room temperature (r.t.) for 4 hours, and subjected to rotary evaporation to remove the solvent to obtain 4-acrylamidophenylboronic acid. Into 40 mL of an aqueous solution containing 4-vinylphenylboronic acid (1480.0 mg, 10.0 mmol), 3-sulfopropyl acrylate potassium salt (1392.0 mg, 6.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white powder P8, the average yield of which was 89%. The chemical reaction process of the above synthetic steps is shown in FIG. 5, where x:y=1:0.6. The synthesized P8 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 7.85, 4.10, 2.83-0.94, indicating that P8 has the chemical structure of the reaction product in FIG. 5.

### Example 9. Synthesis of the Polymer P9 Comprising at Least One Boronic Acid Group Described in the Present Application

The polymer P9 comprising at least one boronic acid group described in the present application, i.e., poly-(vinylboronic acid-r-polyethylene glycol monoacrylate)-1-0.3, the synthetic steps of which are as follows:

Into 40 mL of an aqueous solution containing 4-vinylboronic acid (720.0 mg, 10.0 mmol), polyethylene glycol monoacrylate (weight-average molecular weight 480, 1440.0 mg, 3.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white solid P9, the average yield of which was 89%. The chemical reaction process of the above synthetic steps is shown in FIG. 6, where x:y=1:0.3 and n is an integer greater than or equal to 0. The synthesized P9 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 4.20, 3.55, 2.10-0.94, indicating that P9 has the chemical structure of the reaction product in FIG. 6.

### Example 10. Synthesis of the Polymer P10 Comprising at Least One Boronic Acid Group Described in the Present Application

The polymer P10 comprising at least one boronic acid group described in the present application, i.e., poly-(2-propenylboronic acid-r-polyethylene glycol monoacrylate)-1-0.3, the synthetic steps of which are as follows:

Into 40 mL of an aqueous solution containing 2-propenylboronic acid (860.0 mg, 10.0 mmol), polyethylene glycol monoacrylate (weight-average molecular weight 480, 1440.0 mg, 3.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white solid P10, the average yield of which was 89%. The chemical reaction process of the above synthetic steps is shown in FIG. 7, where x:y=1:0.3 and n is an integer greater than or equal to 0. The synthesized P10 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 4.15, 3.53, 2.20-0.83, indicating that P10 has the chemical structure of the reaction product in FIG. 7.

### Example 11. Synthesis of the Polymer P11 Comprising at Least One Boronic Acid Group Described in the Present Application

The polymer P11 comprising at least one boronic acid group described in the present application, i.e., poly-(3-vinylphenylboronic acid-r-acrylic acid)-1-2, the synthetic steps of which are as follows:

Into 40 mL of an aqueous solution containing 3-vinylphenylboronic acid (1480.0 mg, 10.0 mmol), acrylic acid (1440.0 mg, 20.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white powder P11, the average yield of which was 87%. The chemical reaction process of the above synthetic steps is shown in FIG. 8, where x:y=1:2. The synthesized P11 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 7.80, 7.09, 2.63-1.12, indicating that P11 has the chemical structure of the reaction product in FIG. 8.

### Example 12. Synthesis of the Polymer P12 Comprising at Least One Boronic Acid Group Described in the Present Application

The polymer P1 comprising at least one boronic acid group described in the present application2, i.e., poly-(N-acryloyl-N′-3-fluorophenylboronic acid p-formyl-ethylenediamine-r-polyethylene glycol monoacrylate)-1-0.3, the synthetic steps of which are as follows:

Into a solution of tert-butoxycarbonyl ethylenediamine (1.60 g, 10 mmol) in tetrahydrofuran was added 10 mL triethylamine, and then added dropwise a solution of acryloyl chloride (0.95 g, 10 mmol) in tetrahydrofuran slowly, stirred for 4 hours, subjected to rotary evaporation to remove the solvent, washed with saturated brine, and dried to obtain tert-butoxycarbonyl protected acryloyl ethylamine. Into a solution of tert-butoxycarbonyl protected acrylamido ethylamine (2.42 g, 10 mmol) in dichloromethane (40 mL) was added trifluoroacetic acid (TFA, 20 mL) and stirred for one hour, subjected to rotary evaporation for multiple times to remove trifluoroacetic acid, to obtain acrylamido ethylamine. Into a solution of 4-carboxyl-3-fluorophenylboronic acid (1.84 g, 10 mmol) in tetrahydrofuran were added 1-ethyl-(3-dimethylamino propyl)carbodiimide hydrochloride (1.92 g, 10 mmol) and 1-hydroxybenzotriazole (1.35 g, 10 mmol), stirred for 1 hour, and then added a solution of acrylamido ethylamine (1.14 g, 10 mmol) in tetrahydrofuran, stirred for 12 hours, subjected to rotary evaporation to remove the solvent, washed with saturated brine, to obtain N-acryloyl-N′-3-fluorophenylboronic acid p-formyl-ethylenediamine. Into 40 mL of an aqueous solution containing N-acryloyl-N′-3-fluorophenylboronic acid p-formyl-ethylenediamine (280.0 mg, 10.0 mmol), polyethylene glycol monoacrylate (weight-average molecular weight 480, 1440.0 mg, 3.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white solid P12, the average yield of which was 59%. The chemical reaction process of the above synthetic steps is shown in FIG. 9, where x:y=1:0.3 and n is an integer greater than or equal to 0. The synthesized P12 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 8.01, 7.73, 7.20, 4.25, 3.57, 3.43, 2.95-0.91, indicating that P12 has the chemical structure of the reaction product in FIG. 9.

### Example 13. Synthesis of the Polymer P13 Comprising at Least One Boronic Acid Group Described in the Present Application

The polymer P13 comprising at least one boronic acid group described in the present application, i.e., poly-((5-amino-2-(hydroxymethyl)phenylboronic acid cyclic monoester acrylamide)-r-(3-sulfopropyl acrylate potassium salt))-1-0.6, the synthetic steps of which are as follows:

Into a tetrahydrofuran solution containing (5-amino-2-(hydroxymethyl)phenylboronic acid cyclic monoester (1.63 g, 10 mmol) was added 10 ml triethylamine, and then added dropwise a solution of acryloyl chloride (0.95 g, 10 mmol) in tetrahydrofuran slowly, stirred for 4 hours, and subjected to rotary evaporation to remove the solvent, to obtain 5-amino-2-(hydroxymethyl)phenylboronic acid cyclic monoester acrylamide. Into 40 mL of an aqueous solution containing 5-amino-2-(hydroxymethyl)phenylboronic acid cyclic monoester acrylamide (2.17 g, 10 mmol), 3-sulfopropyl acrylate potassium salt (1.39 g, 6 mmol), sodium bisulfite (94.7 mg, 0.9 mmol), polyethylene glycol (weight-average molecular weight 400, 15.78 g, 39.5 mmol), and sodium dodecyl sulfate (3.95 g, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white powder P13, the average yield of which was 89%. The chemical reaction process of the above synthetic steps is shown in FIG. 10, where x:y=1:0.6. The synthesized P13 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 7.29, 4.08, 2.79-1.54, indicating that P13 has the chemical structure of the reaction product in FIG. 10.

### Example 14. Synthesis of the Polymer P14 Comprising at Least One Boronic Acid Group Described in the Present Application

The polymer P14 comprising at least one boronic acid group described in the present application, i.e., poly-(N-acryloyl-N′-sulfonylphenyl boronic acid ethylenediamine-r-polyethylene glycol monoacrylate)-1-0.3, the synthetic steps of which are as follows:

Into a solution of tert-butoxycarbonyl ethylenediamine (1.60 g, 10 mmol) in tetrahydrofuran was added 10 mL triethylamine, and then added dropwise a solution of acryloyl chloride (0.95 g, 10 mmol) in tetrahydrofuran slowly, stirred for 4 hours, subjected to rotary evaporation to remove the solvent, washed with saturated brine, and dried to obtain tert-butoxycarbonyl protected acrylamido ethylamine. Into tert-butoxycarbonyl protected acrylamido ethylamine (2.42 g, 10 mmol) in dichloromethane (40 mL) was added trifluoroacetic acid (20 mL) and stirred for one hour, subjected to rotary evaporation for multiple times to remove trifluoroacetic acid, to obtain acrylamido ethylamine. Into a solution of 4-sulfonylphenylboronic acid (2.02 g, 10 mmol) in tetrahydrofuran were added 1-ethyl-(3-dimethylamino propyl)carbodiimide hydrochloride (1.92 g, 10 mmol) and 1-hydroxybenzotriazole (1.35 g, 10 mmol), stirred for 1 hour, and then added a solution of acrylamido ethylamine (1.14 g, 10 mmol) in tetrahydrofuran, stirred for 12 hours, subjected to rotary evaporation to remove the solvent, washed with saturated brine, to obtain N-acryloyl-N′-sulfonylphenyl boronic acid ethylenediamine. Into 40 mL of an aqueous solution containing N-acryloyl-N′-sulfonylphenyl boronic acid ethylenediamine (280.0 mg, 10.0 mmol), polyethylene glycol monoacrylate (weight-average molecular weight 480, 1440.0 mg, 3.0 mmol), sodium bisulfite (94.7 mg, 0.9 mmol), polyethylene glycol (weight-average molecular weight 400, 15.78 g, 39.5 mmol), and sodium dodecyl sulfate (3.95 g, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white solid P14, the average yield of which was 62%. The chemical reaction process of the above synthetic steps is shown in FIG. 11, where x:y=1:0.3 and n is an integer greater than or equal to 0. The synthesized P14 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 8.01, 7.83, 4.35-3.22, 2.33-1.02, indicating that P14 has the chemical structure of the reaction product in FIG. 11.

### Comparative Example 1. Synthesis of Polymer D1

Polymer D1 for comparison with the polymer of the present application, i.e., poly-(styrene-r-acrylic acid)-1-2, the synthetic steps of which are as follows:

Into 40 mL of an aqueous solution containing styrene (1041.5 mg, 10.0 mmol), acrylic acid (1440.0 mg, 20.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white powder D1, the average yield of which was 75%. The chemical reaction process of the above synthetic steps is shown in FIG. 12, where x:y=1:2. The synthesized D1 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 7.25, 2.53, 2.0, indicating that D1 has the chemical structure of the reaction product in FIG. 12.

### Comparative Example 2. Synthesis of Polymer D2

Polymer D2 for comparison with the polymer of the present application, i.e., poly-(4-vinylphenylboronic acid-r-acrylic acid)-0.01-1, the synthetic steps of which are as follows:

Into 40 mL of an aqueous solution containing 4-vinylphenylboronic acid (148.0 mg, 1.0 mmol), acrylic acid (1440.0 mg, 20.0 mmol), sodium bisulfite (94.7 mg, 0.9 mmol), polyethylene glycol (weight-average molecular weight 400, 15780.0 mg, 39.5 mmol), and sodium dodecyl sulfate (3950.0 mg, 13.7 mmol) was added dropwise 2 mL of an aqueous solution of sodium persulfate (47.4 mg, 0.2 mmol). They were stirred overnight in an oil bath at 72° C. to react and polymerize. The reaction solution was adjusted to neutral using an aqueous solution of sodium hydroxide at 1 mol/L, and purified and concentrated using an ultrafiltration membrane package (Sartorius Vivoflow 50, MWCO 10000) driven by a peristaltic pump. After then, it was freeze-dried to obtain a white powder D2, the average yield of which was 93%. The chemical reaction process of the above synthetic steps is shown in FIG. 1, where x:y=0.01:1. The synthesized D2 was detected by proton nuclear magnetic resonance spectroscopy ¹HNMR (the parameter is 400 MHz; the deuterated solvent is deuterium oxide, i.e., D₂O; the chemical shift is in ppm), with the peak positions (i.e., characteristic chemical shifts) at 7.50, 6.76, 2.19, 1.72, 1.56, indicating that D2 has the chemical structure of the reaction product in FIG. 1.

### Example 15-53. Effect of the Polymer Comprising at Least One Boronic Acid Group Described in the Present Application in Inhibiting the Degradation of Disaccharides or Polysaccharides by Enzymes in Simulated Intestinal Fluid

The experimental method is as follows:

Disaccharides: A solution of the polymer of the present application or the polymer of the comparative example at a mass percent of 1 wt. %, a solution of disaccharides at 1 wt. % and a solution of α-glucosidase at 0.1 wt. % were respectively formulated with simulated intestinal fluid. An experimental group and a control group were set respectively. For the experimental group: Each 10 mL of the three above solutions were mixed, and reacted in a 37° C. shaker at 200 rpm for 4 hours. For the control group: Each 10 mL of the disaccharide solution and the α-glucosidase solution were mixed, and reacted in a 37° C. shaker at 200 rpm for 4 hours. A glucose assay kit was employed to detect the glucose concentration. 6 parallel samples were set for each group.

Polysaccharides: A solution of the polymer of the present application at a mass percent of 1 wt. %, a solution of polysaccharides at 1 wt. %, and a solution of (0.1 wt. % amylase+0.1 wt. % α-glucosidase) were respectively formulated with simulated intestinal fluid. An experimental group and a control group were set respectively. For the experimental group: Each 10 mL of the above three solutions were mixed, and reacted in a 37° C. shaker at 200 rpm for 4 hours. For the control group: Each 10 mL of the polysaccharide solution and the (amylase+α-glucosidase) solution were mixed, and reacted in a 37° C. shaker at 200 rpm for 4 hours. A glucose assay kit was employed to detect the glucose concentration. 6 parallel samples were set for each group.

The detected glucose concentration is divided by the theoretical glucose concentration, to get the saccharide digestibility. Wherein, the theoretical glucose concentration is calculated by a method as follows:
The theoretical glucose concentration of maltose=Initial maltose concentration;
The theoretical glucose concentration of sucrose=Initial sucrose concentration÷2;
The theoretical glucose concentration of starch=Initial starch concentration;
The theoretical glucose concentration of dextrin=Initial dextrin concentration;
The theoretical glucose concentration of glycogen=Initial glycogen concentration.

The specific parameters and results of the experiment are shown in Table 1. It can be seen that, compared with the control group as well as the polymers D1 and D2 of the comparative examples in the experimental group, the polymers of the present application in the experimental group have lower saccharide digestibility, indicating that the saccharides are more bound by the polymer of the present application and less degraded by enzymes, further indicating that the polymer of the present application has a strong ability to inhibit the degradation of disaccharides or polysaccharides by enzymes, and has the effect of reducing the digestion and absorption of disaccharides or polysaccharides by the body, thereby reducing the blood glucose.

TABLE 1
Specific parameters and results of Examples 15-53

| | Saccharide | | Polymer | | Enzyme | | | |
|---|---|---|---|---|---|---|---|---|
| | | Concentration | | Concentration | | concentration | | |
| Example | Saccharide | (mass | Polymer | (mass | Enzyme | (mass | | Saccharide |
| No. | type | percent) | Name | percent) | Type | percent) | pH | digestibility |
| 15 | Maltose | 1% | None | 0 | α-glucosidase | 0.1% | 6.8 | 85% |
| 16 | Maltose | 2% | | | | | | 82% |
| 17 | Maltose | 5% | | | | | | 79% |
| 18 | Sucrose | 1% | | | α-glucosidase | | | 65% |
| 19 | Dextrin | | | | α-glucosidase + | | | 80% |
| 20 | Glycogen | | | | amylase | | | 50% |
| 21 | Starch | | | | | | | 70% |
| 22 | Maltose | 1% | P1 | 1% | α-glucosidase | 0.1% | | 7% |
| 23 | | 2% | | | | | | 9% |
| 24 | | 5% | | | | | | 13% |
| 25 | Sucrose | 1% | | | | | | 4% |
| 26 | Dextrin | | | | α-glucosidase + | | | 5% |
| 27 | Glycogen | | | | amylase | | | 3% |
| 28 | Starch | | | | | | | 4% |
| 29 | Maltose | 1% | P2 | 1% | α-glucosidase | 0.1% | | 7% |
| 30 | | 2% | | | | | | 4% |
| 31 | | 5% | | | | | | 6% |
| 32 | Sucrose | 1% | | | | | | 5% |
| 33 | Dextrin | | | | α-glucosidase + | | | 7% |
| 34 | Glycogen | | | | amylase | | | 4% |
| 35 | Starch | | | | | | | 6% |
| 36 | Maltose | 1% | P6 | 1% | α-glucosidase | | | 77% |
| 37 | Sucrose | | | | | | | 33% |
| 38 | Dextrin | | | | α-glucosidase + | 0.1% | | 51% |
| 39 | Glycogen | | | | amylase | | | 29% |
| 40 | Starch | | | | | | | 40% |
| 41 | Maltose | 1% | P3 | 1% | α-glucosidase | 0.1% | | 66% |
| 42 | | | P4 | | | | | 5% |
| 43 | | | P5 | | | | | 19% |
| 44 | | | P7 | | | | | 79% |
| 45 | | | P8 | | | | | 79% |
| 46 | | | P9 | | | | | 50% |
| 47 | | | P10 | | | | | 44% |
| 48 | | | P11 | | | | | 23% |
| 49 | | | P12 | | | | | 29% |
| 50 | | | P13 | | | | | 49% |
| 51 | | | P14 | | | | | 30% |
| 52 | | | D1 | | | | | 85% |
| 53 | | | D2 | | | | | 82% |

### Example 54-64. Effect of In Vitro Simulated Gastric Acid Conditions on the Effect of the Polymer of the Present Application in Inhibiting the Degradation of Disaccharides

Experimental method: A solution of the polymer of the present application at a mass percent of 1 wt. %, a solution of disaccharides at 1 wt. % and a solution of α-glucosidase at 0.1 wt. % were respectively formulated with simulated intestinal fluid. A variable pH experimental group, a constant pH experimental group and a control group are set, specifically referring to the pH column in Table 2, wherein the constant pH means that the pH remains constant during the experiment, and the variable pH means that pH changes during the experiment. For the variable pH experimental group: 10 mL of the polymer solution was firstly adjusted to pH 2.0 with 0.8 mmol/L of hydrochloric acid, incubated at a constant temperature of 37° C. for 30 min, then adjusted to pH 6.8 with 1 mmol/L of sodium bicarbonate solution; and then 10 mL of α-glucosidase solution and 10 mL of disaccharide solution were immediately added into the polymer solution at the same time; and after the mixed solution was incubated at 37° C. for 4 hours, a glucose assay kit was employed to detect the glucose concentration. For the constant pH experimental group: Each 10 mL of the above three solutions were mixed, incubated at 37° C. for 4 hours; and a glucose assay kit was employed to detect the glucose concentration. For the control group: Each 10 mL of the disaccharide solution and the α-glucosidase solution were mixed, incubated at 37° C. for 4 hours; and a glucose assay kit was employed to detect the glucose concentration.

The obtained glucose concentration is divided by the theoretical glucose concentration to get the saccharide digestibility, wherein, the calculation method of the theoretical glucose concentration is the same as that used in Examples 15-53, so it will not be repeated here. The specific parameters and results of the experiment are shown in Table 2. The results show that, acidic solution treatment did not reduce the efficiency of the polymer of the present application in inhibiting the saccharide degradation, thus indicating that after gastric acid treatment, the polymer of the present application can quickly restore the ability to inhibit the degradation of disaccharides by enzymes under the action of alkaline intestinal fluid. That is to say, when the polymer of the present application is orally administered, the polymer experiences the pH changes when passing through the gastrointestinal tract, i.e., the polymer firstly enters the acidic stomach, where the polymer does not bind to the saccharide, but the saccharide cannot be absorbed in the stomach, either; then the polymer enters the small intestine, where the pH is elevated, the polymer of the present application quickly restore the ability to bind to the saccharide, thereby effectively inhibiting the degradation of saccharides by corresponding enzymes in the small intestine, further effectively preventing the saccharides from being absorbed by the body.

TABLE 2
The specific parameters and results of the experiment in Examples 54-64

| | Saccharide | | Polymer | | Enzyme | | | |
|---|---|---|---|---|---|---|---|---|
| | | Concentration | | Concentration | | Concentration | | |
| Example | Saccharide | (mass | Polymer | (mass | Enzyme | (mass | | Saccharide |
| No. | type | percent) | Name | percent) | type | percent) | pH | digestibility |
| 54 | Maltose | 1% | None | 0 | α-glucosidase | 0.1% | Constant | 84% |
| 55 | Maltose | 1% | P1 | 1% | α-glucosidase | 0.1% | Constant | 7% |
| 56 | Maltose | 1% | P1 | 1% | α-glucosidase | 0.1% | Variable | 5% |
| 57 | Maltose | 1% | P2 | 1% | α-glucosidase | 0.1% | Constant | 7% |
| 58 | Maltose | 1% | P2 | 1% | α-glucosidase | 0.1% | Variable | 5% |
| 59 | Maltose | 1% | P6 | 1% | α-glucosidase | 0.1% | Constant | 71% |
| 60 | Maltose | 1% | P6 | 1% | α-glucosidase | 0.1% | Variable | 69% |
| 61 | Maltose | 1% | P9 | 1% | α-glucosidase | 0.1% | Constant | 48% |
| 62 | Maltose | 1% | P9 | 1% | α-glucosidase | 0.1% | Variable | 48% |
| 63 | Maltose | 1% | P10 | 1% | α-glucosidase | 0.1% | Constant | 48% |
| 64 | Maltose | 1% | P10 | 1% | α-glucosidase | 0.1% | Variable | 48% |

### Example 65. In Vitro Simulation of the Situation that the Polymer of the Present Application Inhibits the Absorption of Glucose by Human Body

Experimental method: A dialysis bag (Spectral Medicine, MWCO was 3500) was used as a physical barrier to simulate the small intestine wall, and the diffusion of glucose in the dialysis bag to the outside of the dialysis bag simulated the absorption process of glucose by the small intestine wall. An experimental group and a control group were set. For the experimental group: 8 mL of a mixed solution of glucose (300 mg/mL) and the polymer of the present application or the polymer of the comparative example (300 mg/mL) was formulated, the solution was added into the dialysis bag, then the dialysis bag was sealed well and placed into a 50 mL centrifuge tube, 30 mL of sodium polyacrylate solution (300 mg/mL) was added into the centrifuge tube to make the liquid inside and outside the dialysis bag level. For the control group: 8 mL of a mixed solution of glucose (300 mg/mL) and sodium polyacrylate (300 mg/mL) was formulated, the solution was added into the dialysis bag, then the dialysis bag was sealed well and placed into a 50 mL centrifuge tube, 30 mL of sodium polyacrylate solution (300 mg/mL) was added into the centrifuge tube to make the liquid inside and outside the dialysis bag level. The centrifuge tubes were placed in a constant temperature shaker at 37° C. 10 μL of the solutions outside the dialysis bags were taken at the set time points, respectively and diluted by 20 times. A glucose assay kit was then employed to detect the glucose concentration (mg/dL). 6 parallel samples were set for each group. The glucose concentration outside the dialysis bag is plotted against the time, with the glucose concentration of 0 as the baseline, to calculate the area under the curve (AUC, mg/dL*min). The area of the experimental group is divided by the area of the control group to get the relative absorption rate. The lower the value, the lower the percentage of glucose leaking out of the dialysis bag. The P values in the AUC results are determined by the t-test method (Student's t-test), wherein * represents P≤0.05,** represents P≤0.01,*** represents P≤0.001, **** represents P≤0.0001, ns represents no significant differences.

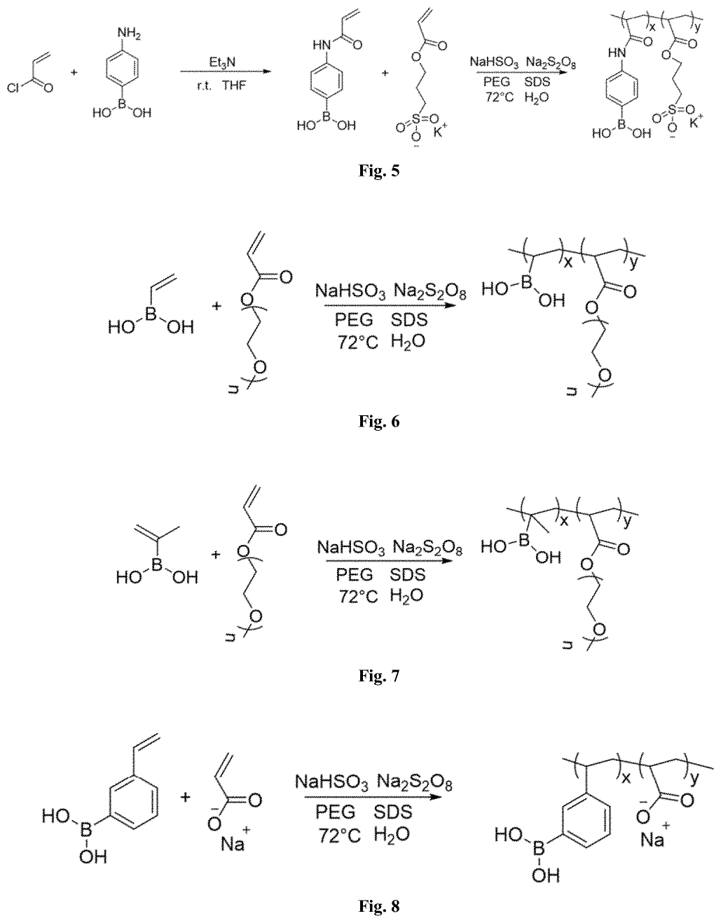

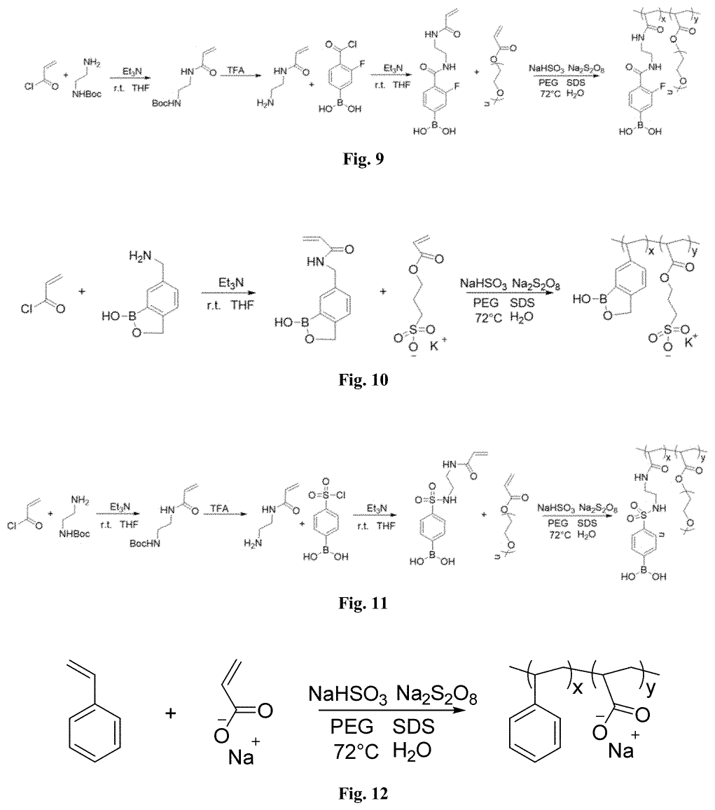

The above experimental results are shown in FIG. 13 and FIG. 14. Wherein, it can be seen from FIG. 13 that, the polymers P1, P2, P10 and P12 of the present application can slow down the diffusion of glucose to the outside of the dialysis bag, while the polymers D1 and D2 of the comparative examples cannot slow down the diffusion of glucose to the outside of the dialysis bag, thus indicating that the polymers of the present application can effectively inhibit the absorption of glucose by the small intestine. It can be seen from FIG. 14 that, the polymers P1, P2, P10 and P12 can significantly reduce the diffusion amount of glucose to the outside of the dialysis bag within 6 hours, while the polymers D1 and D2 of the comparative examples cannot significantly reduce the diffusion amount of glucose to the outside of the dialysis bag.

### Example 66. Acute Toxicological Experiment of the Polymers of the Present Application on C57BL/6J Mice

Experimental method: male C57BL/6J mice (6-8 weeks, about 25 g) were adapted to feeding for one week. The mice were fasted overnight the day before the experiment. The polymers of the present application were gavaged twice a day a dose of 2.5 g/kg for one week. An experimental group and a control group were set. The steps of the control group were the same as those of the experimental group, the only difference was that phosphate buffer saline solution (PBS) was used for each gavage. The survival rate, body weight, food and water intake, and other adverse effects of mice were continuously monitored.

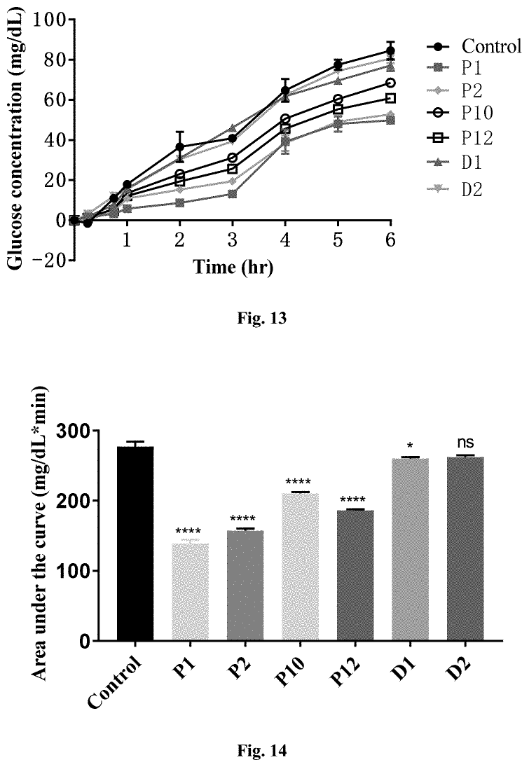

During the experiment, the mice behaved normally without any abnormalities. One week later, the mice were sacrificed and dissected. It was focused on observing whether there were problems such as intestinal obstruction. No abnormalities were observed in the experimental group and the control group. The body weight changes of mice are shown in FIG. 15. It can be seen that, there are no significant differences between the effects of the polymers P1, P2 of the present application on the body weight of mice and the effects of the PBS solution on the body weight of mice, indicating that oral administration of P1, P2 at large doses (5 g/kg/day) in a short time (1 week) will not show acute toxicity.

### Example 67. Effect of the Polymers of the Present Application on the Digestive Tract of C57BL/6J Mice

Experimental method: male C57BL/6J mice (6-8 weeks, about 25 g) were adapted to feeding for one week. The mice were fasted overnight the day before the experiment. An experimental group and a control group were set, the only difference between them was that the drinking water for mice in the experimental group contained the polymer P1 (5 wt. %) of the present application, while the drinking water for mice in the control group did not contain the polymers of the present application. Mice had free access to standard mouse food and drinking water for a week, and the body weight, water intake, food intake were observed, no significant difference was found. And there were no obvious abnormalities observed in mice during the process. After reaching the end of the time, the mice were sacrificed and the digestive tract from the stomach to the large intestine was taken out. The stomach, small intestine, and large intestine were respectively intercepted for tissue sectioning and pathological analysis, and no significant difference was found, indicating that the polymers of the present application would not affect the gastrointestinal tract.

### Example 68. Distribution of the Polymers of the Present Application in C57BL/6J Mice

Experimental method: The polymer P1 of the present application was stained with the coupling near-infrared dye Sulfo-Cy5 to study the biodistribution and elimination of the polymer P1 of the present application in a mouse model.

Into a 2-(N-morpholino)ethanesulfonic acid buffer solution (IVIES, 0.2 M, pH 6.0) of the polymer P1 (1 mmol carboxylic groups), 1-ethyl-(3-dimethylamino propyl)carbodiimide hydrochloride (EDC HCl, 0.1 mmol) and N-hydroxysulfosuccinimide (Sulfo-NHS, 0.1 mmol) was added dropwise a phosphate buffer saline solution (PBS, 0.8 M, pH 7.4) of Sulfo-Cy5-amine (10 μmol), reacted in dark overnight, dialyzed in dark, and freeze-dried in dark to obtain a blue powder sample, i.e., sulfo cyanine 5-labelled polymer P1 sample (Sulfo-Cy5-P1).

Male C57BL/6J mice (6-8 weeks, about 25 g) were adapted to feeding for one week. The mice were fasted overnight the day before the experiment. Sulfo-Cy5-P1 was dissolved in pure water (400 mg/mL). The Sulfo-Cy5-P1 solution was taken to gavage mice at t=0 (the gavage dose was 2.5 g/kg). After that, the mice were anesthetized with isoflurane at each set time point, and a small animal in vivo imaging instrument (IVIS Lumina III) was used for fluorescence imaging to observe the distribution of fluorescently labeled polymers in the mice. The excitation light and the emission light were set to 650 nm and 670 nm, respectively. The mice were euthanized at the set time point, dissected, and the main organs were taken out for in vitro fluorescence imaging.

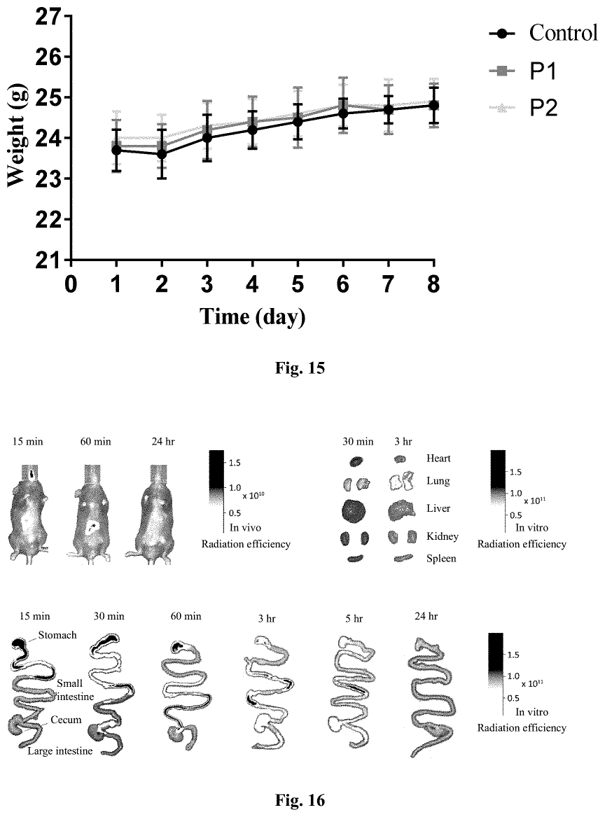

The experimental results are shown in FIG. 16, from which it can be seen that, the polymer P1 of the present application cannot be absorbed after entering the body of mice and can be completely excreted by the mice after 24 hours. In addition, P1 was not detected in various major organs (e.g., heart, lung, liver, kidney, spleen). Therefore, it is indicated that, the polymer P1 of the present application is non-absorbable while exerting a hypoglycemic effect in the body, and it will not enter other organs but only functions in the digestive tract, and can be completely excreted from the body.

### Example 69. Comparison of Oral Glucose Tolerance Test and Intraperitoneal Injection Glucose Tolerance Test in C57BL/6J Mice

Experimental method: Male C57BL/6J mice (6-8 weeks, about 25 g) were adapted to feeding for one week. The mice were fasted overnight the day before the experiment for 12-16 hours. Blood was taken from the tail and a blood glucose meter (Accu-Chek, Roche) was used to test the fasting blood glucose. The oral glucose tolerance experimental method was as follows: mice were divided into two groups equally (an experimental group and a control group), with 8-12 mice in each group. The only difference between the experimental group and the control group was that, mice in the experimental group were gavaged with the polymer P1 of the present application, while mice in the control group were gavaged with phosphate buffer saline solution (PBS). Mice in both the experimental group and the control group were gavaged with a glucose solution 15 min later. It should be noted that the above-mentioned polymer P1 of the present application and glucose were both dissolved in PBS, the doses of which were 1.5 g per kg mouse and 2 g per kg mouse respectively; and the gavage volume of each substance (i.e., the polymer of the present application, PBS, glucose) per mouse was fixed at 0.2 mL. At 15, 30, 60, 90, 120 min after the glucose gavage, a drop of blood was collected from the tail vein of the mice respectively, and the blood glucose level was tested with a blood glucose meter (Accu-Chek, Roche). The data points of blood glucose level are plotted over time, and the area under the curve (AUC) is calculated with the fasting blood glucose level as the baseline. The P values in the blood glucose curve are determined by two-way ANOVA, wherein * represents P≤0.05, ** represents P≤0.01, *** represents P≤0.001, **** represents P≤0.0001, ns represents not significant. The P values in the AUC results are determined by the t-test method (Student's t-test), wherein * represents P≤0.05, ** represents P≤0.01, *** represents P≤0.001, **** represents P≤0.0001, ns represents not significant. In the intraperitoneal injection glucose tolerance test, the treatment of mice was the same as that in the oral glucose tolerance test. The difference was that the glucose solution was injected intraperitoneally, and the changes in blood glucose level were also monitored at the same time points within 2 h.

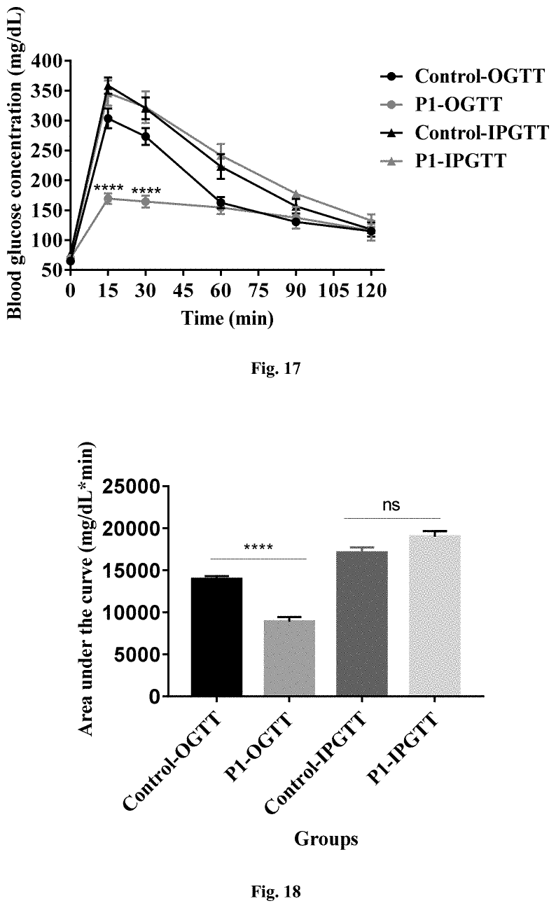

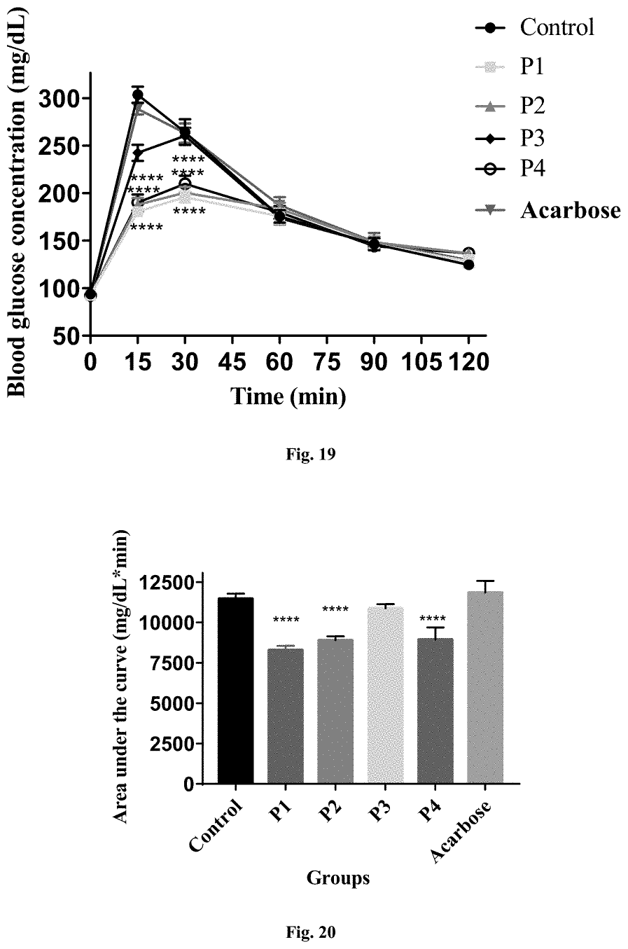

The results of the oral glucose tolerance test (OGTT) and the intraperitoneal glucose tolerance test (IPGTT) are shown in FIG. 17 and FIG. 18. It can be seen that, in the OGTT test, the blood glucose of mice in the experimental group (i.e., P1-OGTT) at 15 min and 30 min was significantly lower than that of the control group (i.e., control-OGTT); while in the IPGTT test, there was almost no difference between the blood glucose levels of the experimental group (i.e., P1-IPGTT) and the control group (i.e., control-IPGTT). This result indicates that the effect of the polymer P1 of the present application on reducing the blood glucose after oral administration of glucose is produced by acting with saccharides, rather than acting with the body, that is, it is a non-systemic effect.

### Example 70. Oral Glucose Tolerance Test of C57BL/6J Mice

Experimental method: Healthy male C57BL/6J mice (6-8 weeks, about 25 g) were adapted to feeding for one week. The mice were fasted overnight the day before the experiment for 12-16 hours. Blood was taken from the tail and a blood glucose meter (Accu-Chek, Roche) was used to test the fasting blood glucose. Mice were divided into three groups equally, i.e., an experimental group, an acarbose group and a control group, with 8-12 mice in each group. The only difference between the three groups was that, mice in the experimental group were gavaged with the polymer of the present application, mice in the acarbose group were gavaged with acarbose, and mice in the control group were gavaged with phosphate buffer saline solution (PBS). Mice in the three groups were all gavaged with a glucose solution 15 min later. It should be noted that the above-mentioned polymer of the present application, acarbose and glucose were all dissolved in PBS, the doses of which were 1.5 g per kg mouse, 10 mg per kg mouse and 2 g per kg mouse respectively; and the gavage volume of each substance (i.e., the polymer of the present application, acarbose, PBS, glucose) per mouse was fixed at 0.2 mL. At 15, 30, 60, 90, 120 min after the glucose gavage, a drop of blood was collected from the tail vein of the mice respectively, and the blood glucose level was tested with a blood glucose meter (Accu-Chek, Roche). The data points of blood glucose level are plotted over time, and the area under the curve (AUC) is calculated with the fasting blood glucose level as the baseline. The P values in the blood glucose curve are determined by two-way ANOVA, wherein * represents P≤0.05, ** represents P≤0.01, *** represents P≤0.001, **** represents P≤0.0001, ns represents not significant. The P values in the AUC results are determined by one-way ANOVA, wherein * represents P≤0.05, ** represents P≤0.01, *** represents P≤0.001, **** represents P≤0.0001, ns represents not significant.

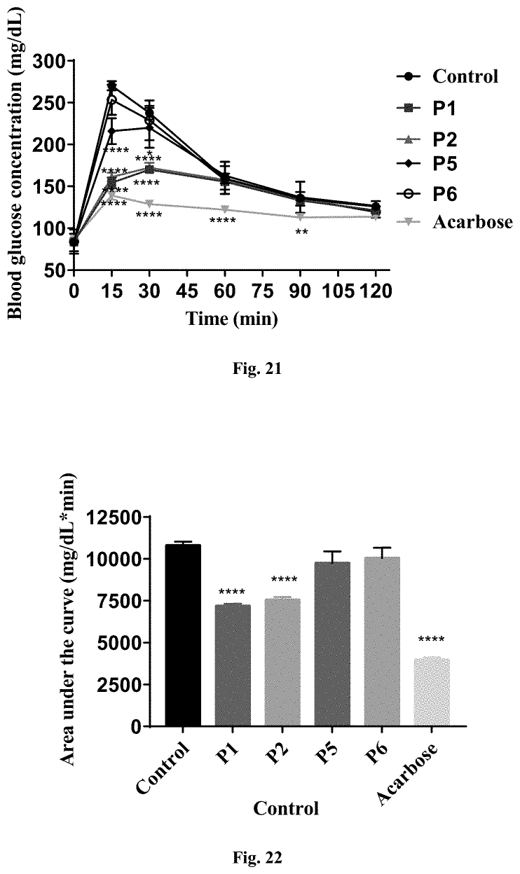

The results of the oral glucose tolerance test are shown in FIG. 19 and FIG. 20. It can be seen from FIG. 19 that, the blood glucose of the polymers P1, P2 and P4 of the present application at 15 min and 30 min was significantly lower than that of the control group; while there is almost no difference in blood glucose between the acarbose group and the control group. In addition, the results of the area under the curve shown in FIG. 20 semi-quantitatively show the hypoglycemic effect of the polymers of the present application. This result indicates that the polymers P1, P2, and P4 of the present application have the effect of reducing the blood glucose after oral administration of glucose.

### Example 71. Oral Maltose Tolerance Test of C57BL/6J Mice

The experimental steps of the oral maltose tolerance test (OMTT) are similar to those of Example 70, except that the gavage after 15 min is not a glucose solution, but a maltose solution. Therefore, the specific experimental steps will not be repeated here.

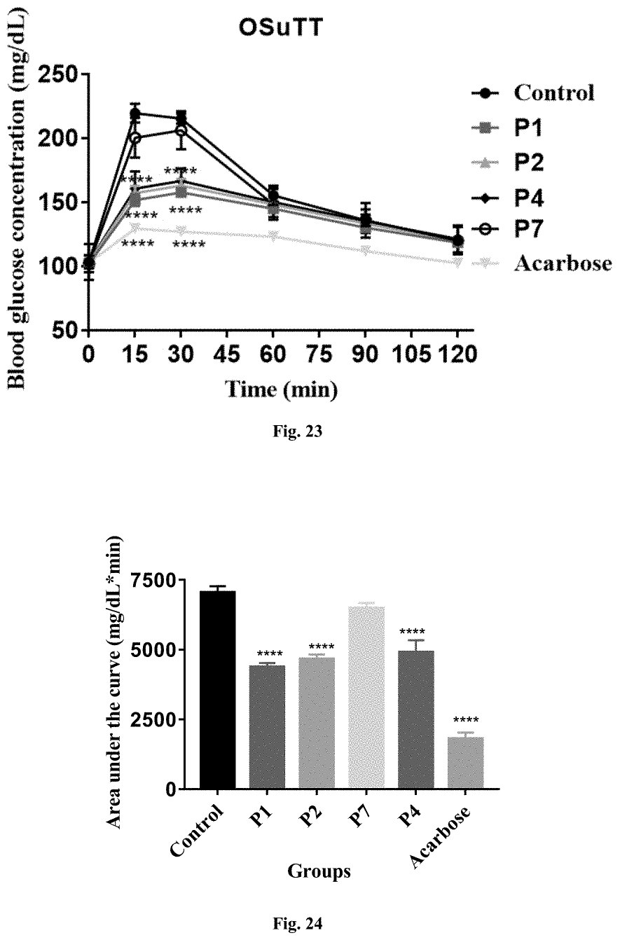

The results of the oral maltose tolerance test (OMTT) are shown in FIG. 21 and FIG. 22. It can be seen from FIG. 21 that, in the OMTT test, the blood glucose of the polymers P1 and P2 of the present application at 15 min and 30 min was significantly lower than that of the control group. Combined with the results shown in FIG. 22, it is illustrated that the polymers P1 and P2 of the present application have the effect of reducing blood glucose after oral administration of maltose.

### Example 72. Oral Sucrose Tolerance Test of C57BL/6J Mice

The experimental steps of the oral sucrose tolerance test (OSuTT) are similar to those of Example 70, except that the gavage after 15 min is not a glucose solution, but a sucrose solution. Therefore, the specific experimental steps will not be repeated here.

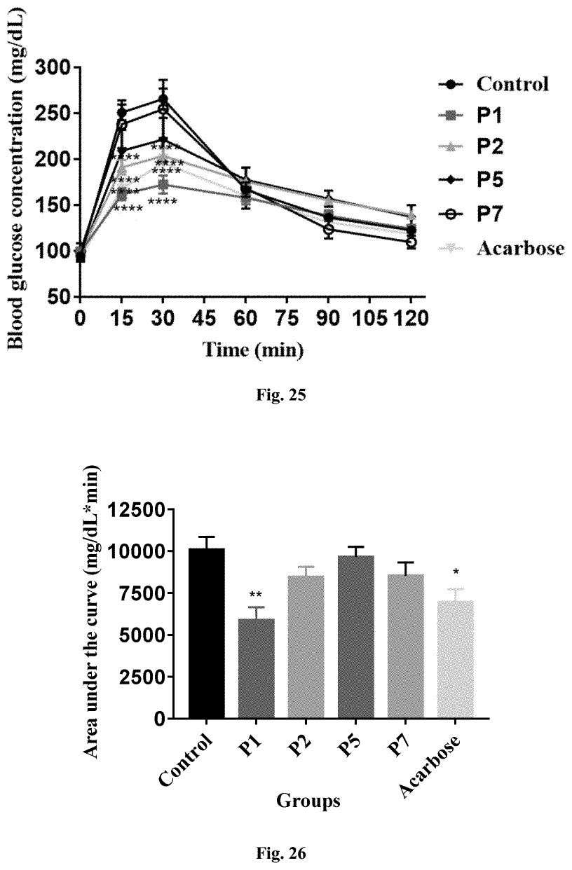

The results of the oral sucrose tolerance test (OSuTT) are shown in FIG. 23 and FIG. 24. It can be seen from FIG. 23 that, in the OSuTT test, the blood glucose of the polymers P1, P2 and P4 of the present application at 15 min and 30 min was significantly lower than that of the control group. Combined with the results shown in FIG. 24, it is illustrated that the polymers P1, P2 and P4 of the present application have the effect of reducing blood glucose after oral administration of sucrose.

### Example 73. Oral Dextrin Tolerance Test of C57BL/6J Mice

The experimental steps of the oral dextrin tolerance test (ODTT) are similar to those of Example 70, except that the gavage after 15 min is not a glucose solution, but a dextrin solution. Therefore, the specific experimental steps will not be repeated here.

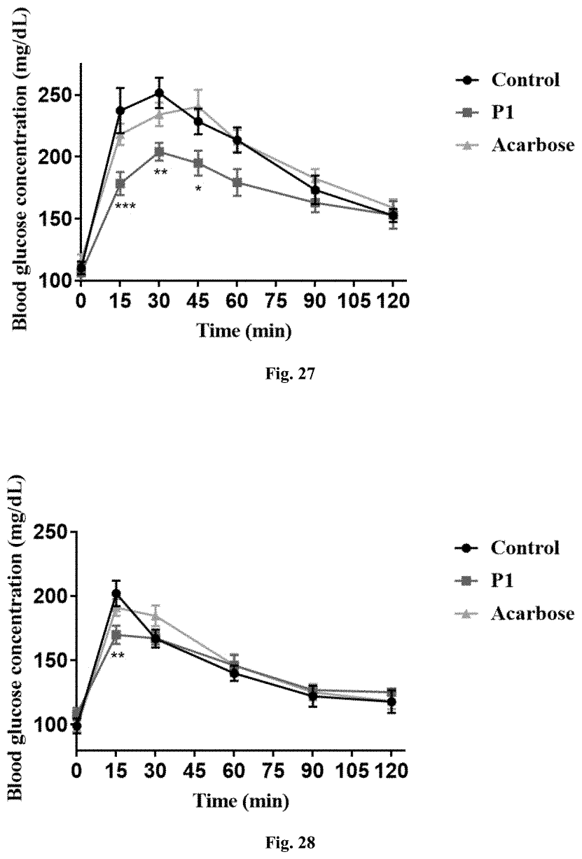

The results of the oral dextrin tolerance test (ODTT) are shown in FIG. 25 and FIG. 26. It can be seen from FIG. 25 that, in the ODTT test, the blood glucose of the polymer P1 of the present application at 15 min and 30 min was significantly lower than that of the control group. Combined with the results shown in FIG. 26, it is illustrated that the polymers P1 of the present application have the effect of reducing blood glucose after oral administration of dextrin.

### Example 74. Oral Real Food Tolerance Test of C57BL/6J Mice

Experimental method: Healthy male C57BL/6J mice (6-8 weeks, about 25 g) were adapted to feeding for one week. The mice were fasted overnight the day before the experiment for 12-16 hours. Blood was taken from the tail and a blood glucose meter (Accu-Chek, Roche) was used to test the fasting blood glucose. Mice were divided into three groups equally, i.e., an experimental group, an acarbose group and a control group, with 8-12 mice in each group. The only difference between the three groups was that, mice in the experimental group were gavaged with the polymer of the present application, mice in the acarbose group were gavaged with acarbose, and mice in the control group were gavaged with phosphate buffer saline solution (PBS). Mice in the three groups were all gavaged with real food homogenate 15 min later. It should be noted that the above-mentioned polymer of the present application and acarbose were both dissolved in PBS, the doses of which were 1.5 g per kg mouse and 10 mg per kg mouse respectively; and the gavage volumes of the polymer of the present application, acarbose, and PBS per mouse were all fixed at 0.2 mL. The above-mentioned real food comprises Chobe blueberry jam, classic Coca Cola and rice gruel. The types, contents and gavage volumes of saccharides contained in them were shown in Table 3, and they were used directly after homogenization. At 15, 30, 60, 90, 120 min after the gavage of real food homogenate, a drop of blood was collected from the tail vein of the mice respectively, and the blood glucose level was tested with a blood glucose meter (Accu-Chek, Roche). The data points of blood glucose level are plotted over time. The elevated blood glucose value of the mice after oral administration of real food is calculated by subtracting the fasting blood glucose value from the highest blood glucose value during the test period. The P values in the blood glucose curve are determined by two-way ANOVA, wherein * represents P≤0.05, ** represents P≤0.01, *** represents P≤0.001, **** represents P≤0.0001. The P values in the elevated blood glucose value results are determined by one-way ANOVA, wherein * represents P≤0.05, ** represents P≤0.01, *** represents P≤0.001, **** represents P≤0.0001.

TABLE 3
Type, content and gavage volume of saccharides
in real food of Example 74

| | Blueberry jam | Coca Cola | Rice gruel |
|---|---|---|---|
| Saccharide type | High fructose | High fructose | Starch |
| | syrup, sucrose | syrup, sucrose | |
| Saccharide | 62.5 | 10.6 | 10.0 |
| content/wt. % | | | |
| Gavage volume/mL | 0.2 | 0.4 | 0.2 |

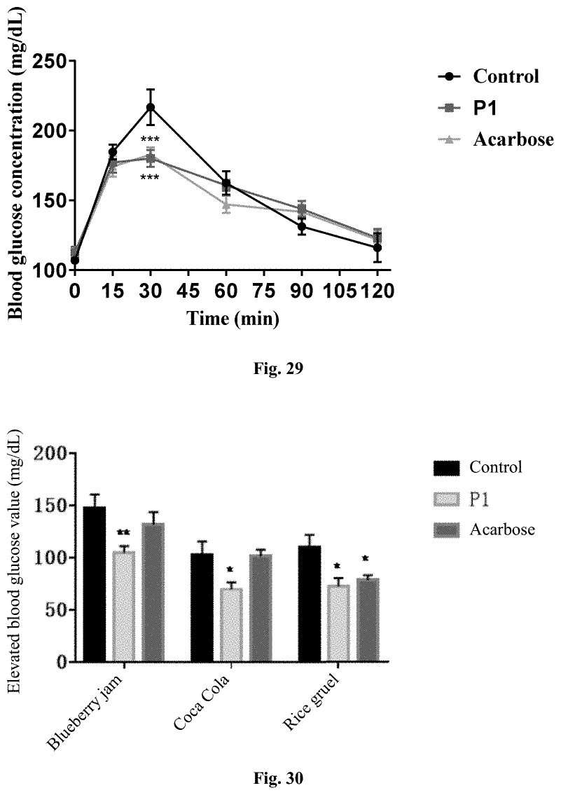

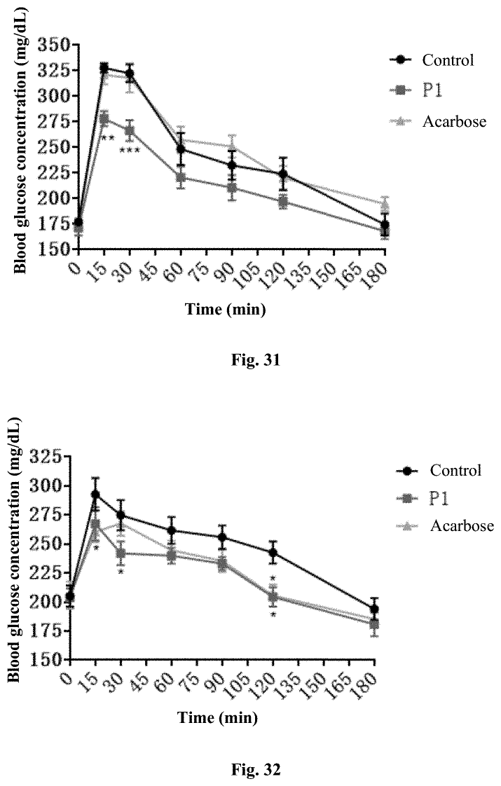

The experimental results are shown in FIG. 27, FIG. 28, FIG. 29 and FIG. 30. Wherein, FIG. 27-FIG. 29 show the blood glucose concentration of mice after oral administration of blueberry jam, Coca Cola and rice gruel, respectively, and FIG. 30 shows the elevated blood glucose value of mice after oral administration of real food.

It can be seen from FIG. 27 that, the blood glucose of the polymer P1 of the present application at 15 min, 30 min, 45 min was significantly lower than that of the control group. This result indicates that the polymer P1 of the present application has the effect of reducing the blood glucose after eating the blueberry jam, and its hypoglycemic effect was better than that of acarbose.

It can be seen from FIG. 28 that, the blood glucose of the polymer P1 of the present application at 15 min was significantly lower than that of the control group. This result indicates that the polymer P1 of the present application has the effect of reducing the blood glucose after drinking Cola, and its hypoglycemic effect is better than that of acarbose.

It can be seen from FIG. 29 that, the blood glucose of the polymer P1 of the present application at 15 min was significantly lower than that of the control group. This result indicates that the polymer P1 of the present application has the effect of reducing the blood glucose after eating the gruel.

It can be seen from FIG. 30 that, for food whose main saccharides are monosaccharides, e.g., Cola and blueberry jam, the hypoglycemic effect of the polymer P1 of the present application was significantly better than that of acarbose; while for food whose main saccharides are polysaccharides, e.g., gruel, the polymer of the present application P1 has a similar hypoglycemic effect to that of acarbose.

### Example 75. Oral Carbohydrate Tolerance Test of Mice with Diet-Induced Obesity (DIO)

The experimental steps of this example are similar to those of Example 70. The carbohydrates used in the experiment are specifically glucose, sucrose, maltose and dextrin. The difference is that the experiment of this example was aimed at mice with diet-induced obesity (DIO) instead of healthy mice. Specifically, male DIO mice (16 weeks, about 45 g) were chosen and adapted to feeding for one week. In addition, in this example, the doses of the polymer of the present application, acarbose and carbohydrates were 1 g per kg mouse, 10 mg per kg mouse, and 1 g per kg mouse. Therefore, the specific experimental steps will not be repeated here.

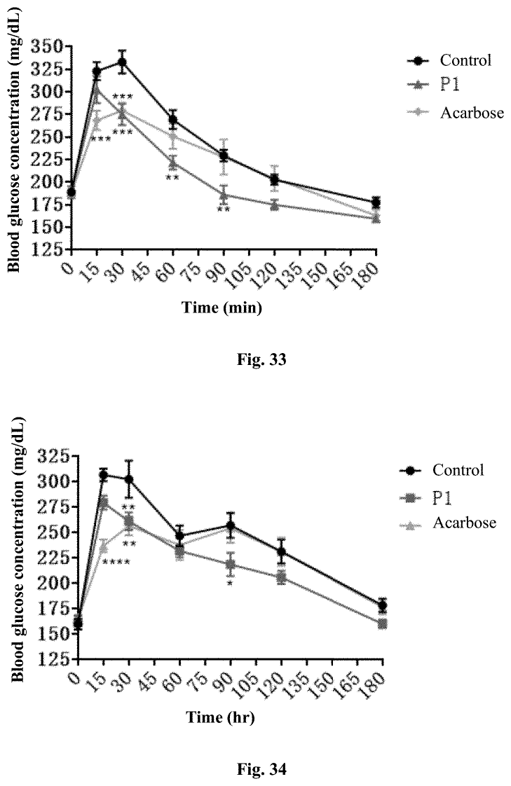

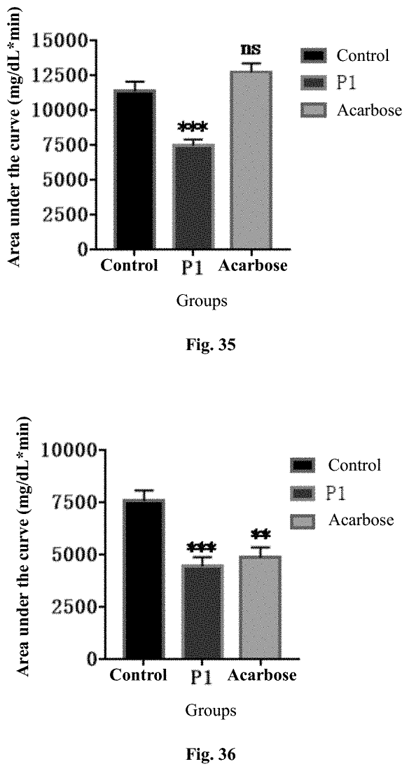

The experimental results are shown in FIGS. 31-38, wherein, FIGS. 31-34 show the blood glucose concentrations of mice with diet-induced obesity after oral administration of glucose, sucrose, maltose and dextrin, respectively; FIGS. 35-38 show the area under the curve (AUC) of mice with diet-induced obesity after oral administration of glucose, sucrose, maltose and dextrin, respectively.

It can be seen from FIGS. 31-38 that, for DIO mice that mimic type II diabetes, the polymer P1 of the present application had the effect of significantly reducing the postprandial blood glucose of oral carbohydrates (such as glucose, sucrose, maltose and dextrin), and the effects are better than those of the acarbose group.

### Example 76. Oral Real Food Tolerance Test of Mice with Diet-Induced Obesity (DIO)

Experimental method: Male mice with diet-induced obesity (DIO) (16 weeks, about 45 g) were adapted to feeding for one week. The mice were fasted overnight the day before the experiment for 12-16 hours. Blood was taken from the tail and a blood glucose meter (Accu-Chek, Roche) was used to test the fasting blood glucose. Mice were divided into three groups equally, i.e., an experimental group, an acarbose group and a control group, with 8-12 mice in each group. The only difference between the three groups was that, mice in the experimental group were gavaged with the polymer of the present application, mice in the acarbose group were gavaged with acarbose, and mice in the control group were gavaged with phosphate buffer saline solution (PBS). Mice in the three groups were all gavaged with real food homogenate 15 min later. It should be noted that the above-mentioned polymer of the present application and acarbose were both dissolved in PBS, the doses of which were 1.0 g per kg mouse and 10 mg per kg mouse respectively; and the gavage volumes of each substance (i.e., the polymer of the present application, acarbose, and PBS) per mouse were all fixed at 0.2 mL. The above-mentioned real food comprised Chobe blueberry jam, classic Coca Cola and rice gruel, which were used directly after being diluted with PBS and homogenized, and the types and contents, the dilution volume multiples and the gavage volumes of saccharides contained in them are seen in Table 4. At 15, 30, 60, 90, 120 min after the gavage of real food homogenate, a drop of blood was collected from the tail vein respectively, and the blood glucose level was tested with a blood glucose meter (Accu-Chek, Roche). The data points of blood glucose level are plotted over time. The elevated blood glucose value of the mice after oral administration of real food is calculated by subtracting the fasting blood glucose value from the highest blood glucose value during the test period. The P values in the blood glucose curve are determined by two-way ANOVA, wherein * represents P≤0.05, ** represents P≤0.01, *** represents P≤0.001, **** represents P≤0.0001. The P values in the elevated blood glucose value results are determined by one-way ANOVA, wherein * represents P≤0.05, ** represents P≤0.01, *** represents P≤0.001, **** represents P≤0.0001.

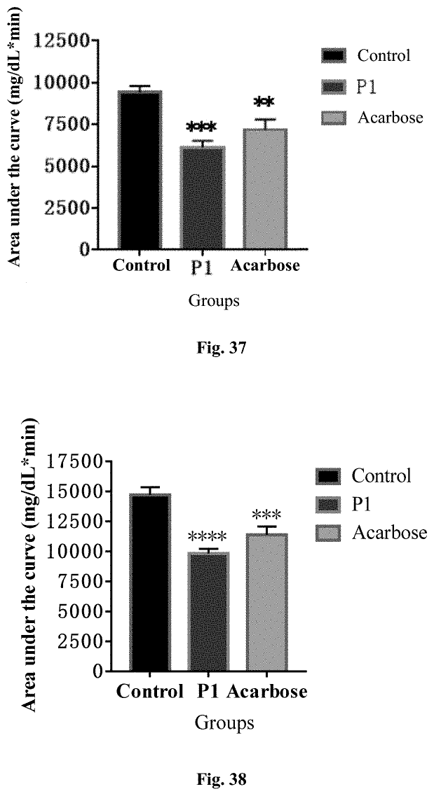

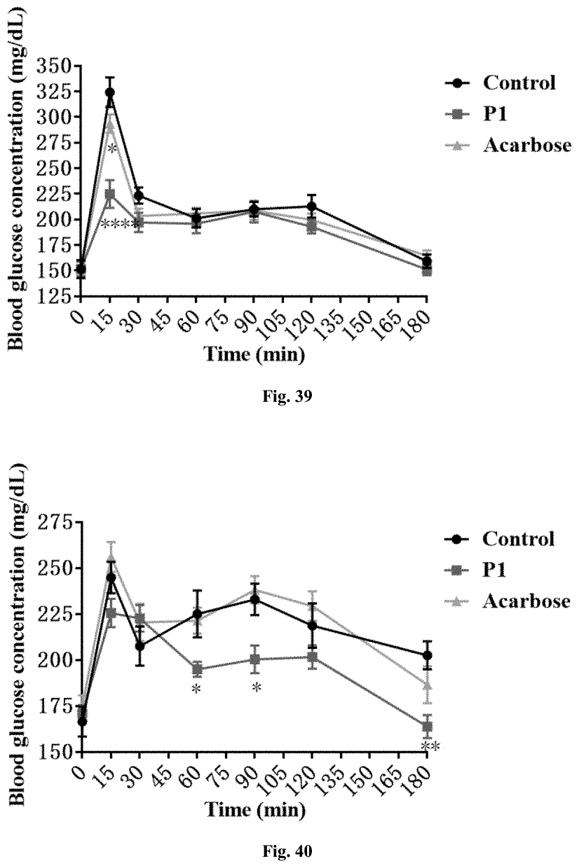

The experimental results are shown in FIGS. 39-42, wherein, FIGS. 39-41 show the blood glucose concentration of mice with diet-induced obesity after oral administration of blueberry jam, Coca Cola, and rice gruel, respectively, FIG. 42 shows the elevated blood glucose value of mice with diet-induced obesity after oral administration of real food.

It can be seen from FIGS. 39-42 that, for DIO mice that mimic type II diabetes, the polymer P1 of the present application has the effect of significantly reducing the blood glucose after oral administration of high-carbohydrate food (such as blueberry jam, Coca Cola and rice gruel), and the effects are better than those of the acarbose group.

TABLE 4
Types and contents, the dilution volume multiples and the gavage
volumes of saccharides contained in real food in Example 76

| | Blueberry jam | Coca Cola | Rice gruel |
|---|---|---|---|
| Saccharide type | High fructose | High fructose | Starch |
| | syrup, sucrose | syrup, sucrose | |
| Saccharide content/wt. % | 62.5 | 10.6 | 10.0 |
| Dilution volume multiple | 4 | 1 (i.e., not | 4 |
| (Volume after dilution/ | | diluted) | |
| Volume before dilution) | | | |
| Gavage volume/mL | 0.2 | 0.2 | 0.2 |

### Example 77. Oral Glucose Tolerance Test of Mice with Streptozotocin (STZ)—Induced Type I Diabetes

The experimental method of this example is similar to that of Example 70. The difference is only in that the experiment of this example is aimed at Streptozotocin-induced mice instead of healthy mice. Specifically, male STZ mice (16 weeks, about 45 g) were chosen and adapted to feeding for one week. Therefore, the specific experimental steps will not be repeated here. The area under the curve (AUC) is calculated with the lowest blood glucose value of each group at the last time point as the baseline. The P values in the AUC results are determined by one-way ANOVA, wherein * represents P≤0.05, ** represents P≤0.01, *** represents P≤0.001, **** represents P≤0.0001, ns represents not significant.

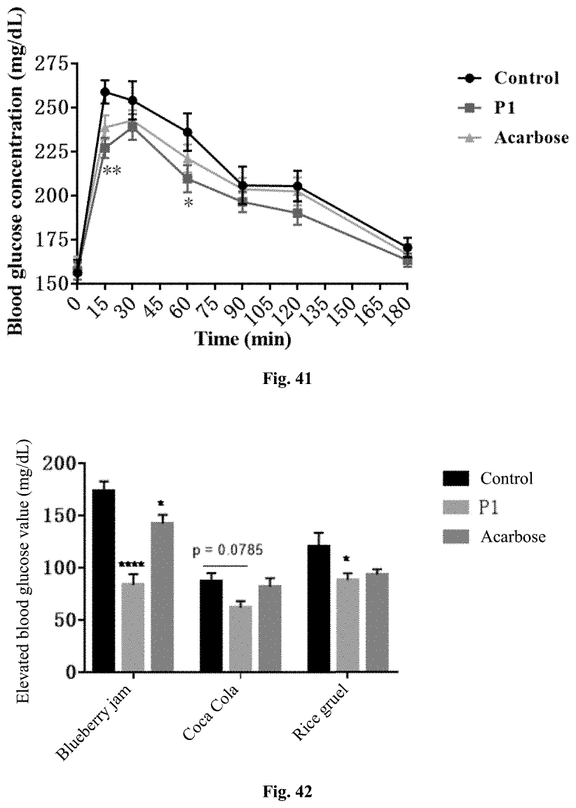

The experimental results are shown in FIG. 43 and FIG. 44, wherein, in FIG. 44, the panel PBS represents the control group. It can be seen from FIG. 43 and FIG. 44 that, for STZ-induced mice that mimic type I diabetes, the polymer P1 of the present application had the effect of significantly reducing the blood glucose after oral administration of glucose.

### Example 78. Early Model of Steatohepatitis of Fructose-Induced C57BL/6J Mice

Experimental method: male C57BL/6J mice (6-8 weeks, about 25 g) were adapted to feeding for one week. The mice were fasted overnight the day before the experiment. Mice were divided into three groups equally, the blank group, the fructose group and the prevention group. The drinking water for the blank group was normal drinking water, the drinking water for the fructose group was fructose solution (concentration of 20 wt. %), and the drinking water for the prevention group was a mixed solution of fructose solution (concentration of 20 wt. %) and the polymer of the present application (P1, concentration of 5 wt. %). The three groups of mice drank freely for 15 days. After 15 days, the mice were sacrificed, and the liver was taken out for biochemical and histological analysis. For biochemical testing, the liver homogenate was extracted, and the contents of total cholesterol, triglycerides and free fatty acids in the liver were detected using the corresponding kit. The normalized value of the three sets of experimental results were calculated. Among them, the normalized value of each group=the experimental result of each group/the experimental result of the blank group. For example, the normalized value of total cholesterol in the fructose group=the content of total cholesterol in the fructose group/the content of total cholesterol in the blank group. For another example, the normalized value of triglycerides in the prevention group=the content of triglycerides in the prevention group/the content of triglycerides in the blank group. For yet another example, the normalized value of free fatty acids in the blank group=the content of free fatty acids in the blank group/the content of free fatty acids in the blank group, i.e., equals to 1. P values are determined by one-way ANOVA, wherein * represents P≤0.05, ** represents P≤0.01, *** represents P≤0.001, **** represents P≤0.0001. In histological analysis, the liver was frozen and sectioned, and stained with Oil Red O to observe the accumulation of triglycerides in the liver.

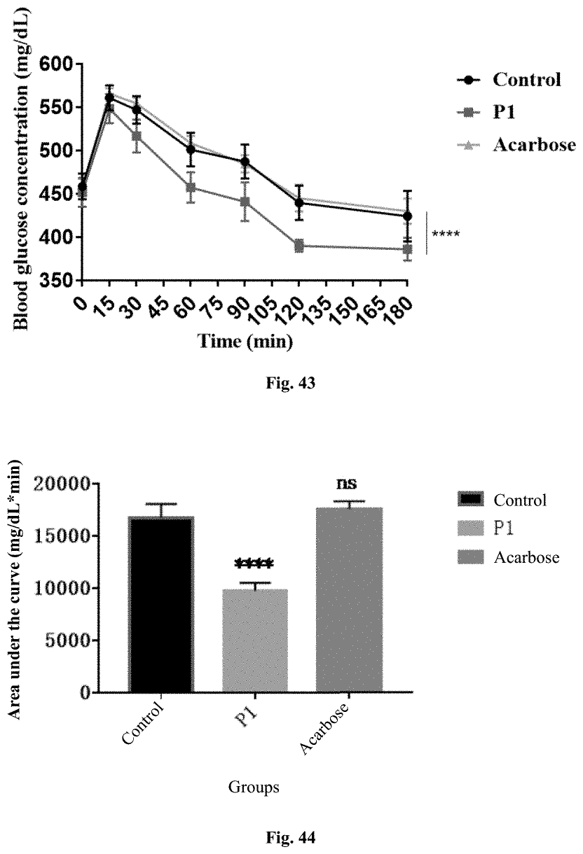

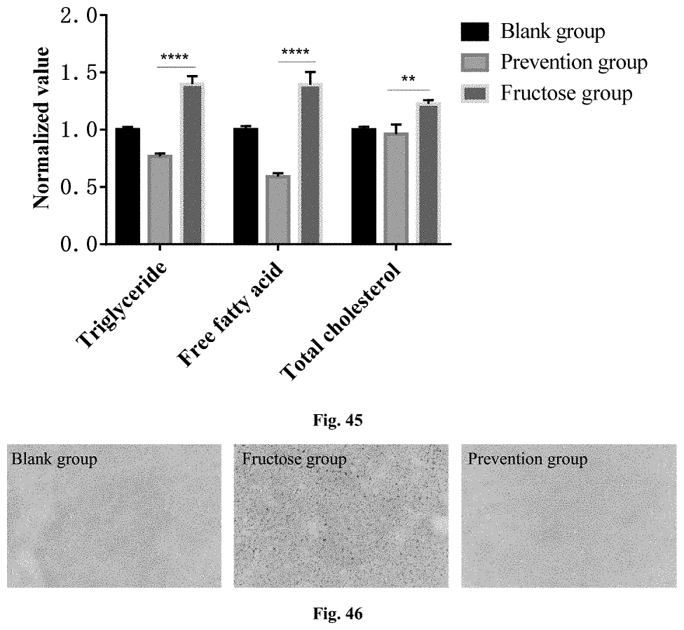

The experimental results are shown in FIG. 45 and FIG. 46. Wherein, FIG. 45 shows the relative contents of total cholesterol, triglycerides and free fatty acids in the livers of the three groups of mice. FIG. 46 shows the optical micrographs of the liver sections of the three groups of mice after being stained with Oil Red O, wherein the model of the optical microscope is Nikon NI-E upright microscope.

It can be seen from FIG. 45 that, the contents of total cholesterol, triglycerides and free fatty acids in the livers of mice in the prevention group are significantly lower than the contents of total cholesterol, triglycerides and free fatty acids in the fructose group. It can be seen from FIG. 46 that, the liver lipids (black particles) of mice in the prevention group are significantly less than those in the fructose group, indicating that the polymer of the present application has a preventive effect on early fatty hepatitis induced by fructose.

The foregoing detailed description is provided by way of explanation and examples, and is not intended to limit the scope of the appended claims. Various changes of the embodiments listed in the present application are obvious to a person of ordinary skill in the art, and are reserved within the scope of the appended claims and their equivalents.

What is claimed is:

1. A method for treating or preventing saccharide-related diseases or disorders, comprising administering a therapeutically or prophylactically effective amount of a polymer comprising at least one boronic acid group to a subject having or at risk of having the saccharide-related diseases or disorders.

2. A method for reducing a saccharide level of a subject, comprising administering a therapeutically or prophylactically effective amount of a polymer comprising at least one boronic acid group to the subject.

3. (canceled)

4. (canceled)

5. (canceled)

6. The method according to claim 1, wherein the polymer comprising at least one boronic acid group is administered as a pharmaceutically active ingredient.

7. The method according to claim 6, wherein the pharmaceutical activity comprises that, compared with a control group, the proportion of the saccharide that has been degraded enzymatically in the subject to whom the polymer comprising at least one boronic acid group is administered is reduced, and the control group is the subject to whom the polymer comprising at least one boronic acid group is not administered.

8. The method according to claim 7, wherein the enzymatic hydrolysis comprises enzymatic hydrolysis by a related saccharidase, and the related saccharidase comprises a glycosylase.

9. The method according to claim 1, wherein the polymer comprising at least one boronic acid group interacts with the saccharide directly.

10. The method according to claim 1, wherein before, simultaneously and/or after the administration of the polymer comprising at least one boronic acid group, other hypoglycemic drugs are administered to the subject.

11. The method according to claim 10, wherein the other hypoglycemic drugs are selected from insulin and the analogue thereof, insulin secretagogue, metformin drug, α-glucosidase inhibitor, insulin sensitizer, peroxisome proliferator-activated receptor agonist (PPAR agonist), GPR40 agonist, JNK inhibitor, pan-AMPK activator, incretin analogue, glucokinase agonist (GKA), G protein-coupled receptor agonist (GPCR agonists), SGLT1 inhibitor, SGLT2 inhibitor, DPP-4 inhibitor, glucagon receptor agonist (GCGR agonist), GIP receptor agonist, GSK-3 inhibitor, amylin analogue, vanadium-containing compound, GFAT inhibitor, 11β-HSD1 inhibitor, Sirtuin-1 (SIRT-1) agonist, PTP1B inhibitor, PI3K agonist, GLP-2 receptor agonist, and/or GLP-1 receptor agonist.

12. The method according to claim 1, wherein the saccharide is selected from: monosaccharide, disaccharide, polysaccharide, and/or substance comprising the monosaccharide, the disaccharide and/or the polysaccharide.

13. The method according to claim 1, wherein the administration is oral administration.

14. The method according to claim 1, wherein the polymer comprising at least one boronic acid group is formulated as an oral preparation.

15. The method according to claim 1, wherein the saccharide-related diseases or disorders are selected from: obesity, diabetes and/or fatty liver.

16. The method according to claim 1, wherein the polymer comprising at least one boronic acid group has a structure represented by Formula I, 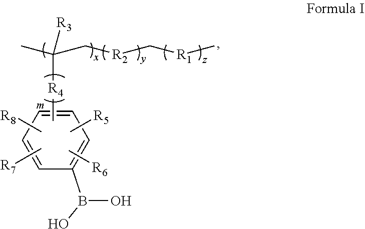
Wherein, R1 or R2 is selected from the following structures and salts thereof: 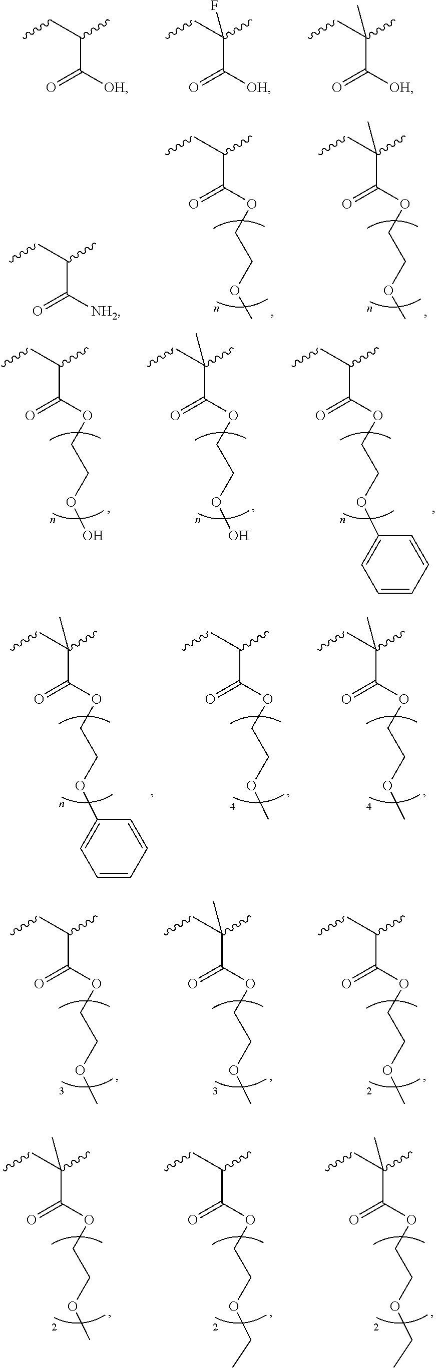 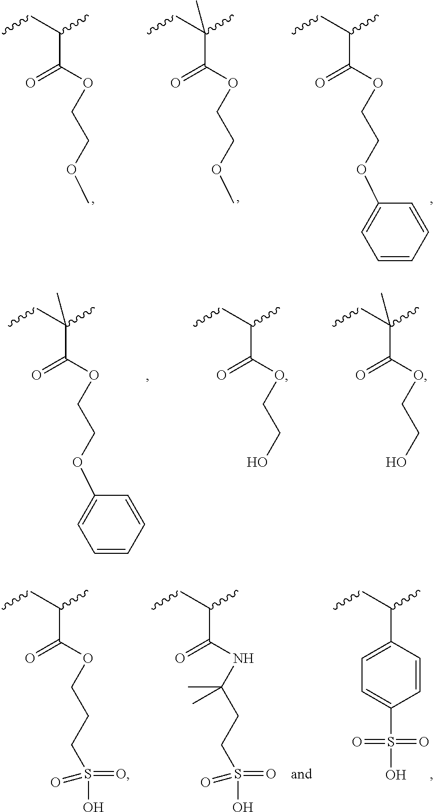
wherein n is an integer greater than or equal to 0;
R3 is selected from the following structures: 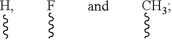
R4 is selected from the following structures: 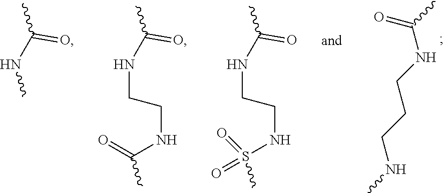
R5 or R6 or R7 or R8 is selected from the following structures: 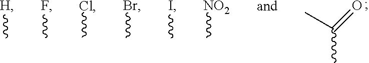
m is an integer greater than or equal to 0, x is a positive integer greater than or equal to 1, y and z are integers greater than or equal to 0, and when y is not equal to 0, x:y=1:(0.000001-90); when z is not equal to 0, x:z=1:(0.000001-90);
Alternatively, the polymer has a structure represented by Formula II, 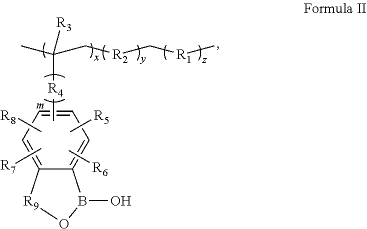
Wherein, R1 or R2 is selected from the following structures and salts thereof: 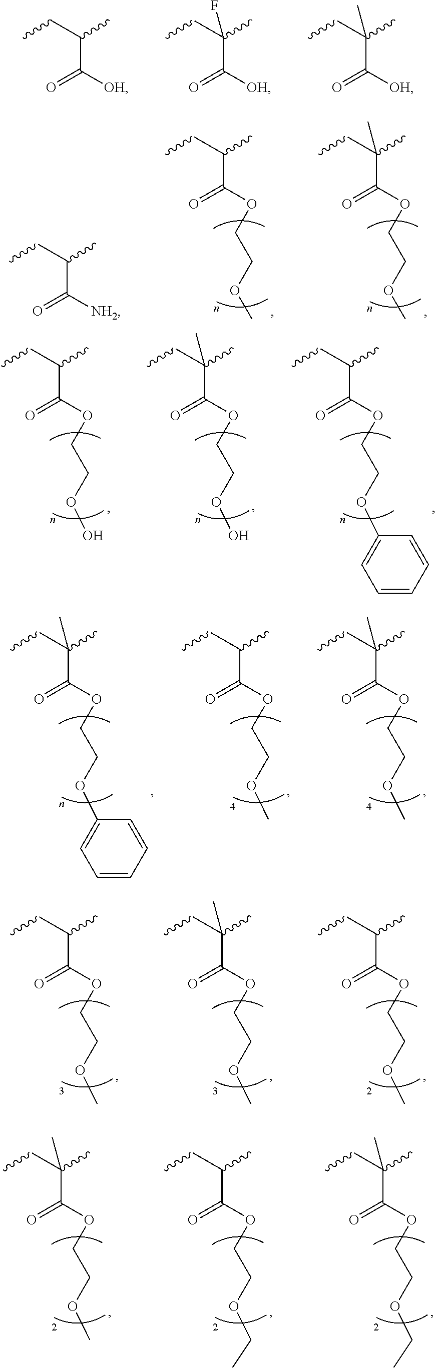 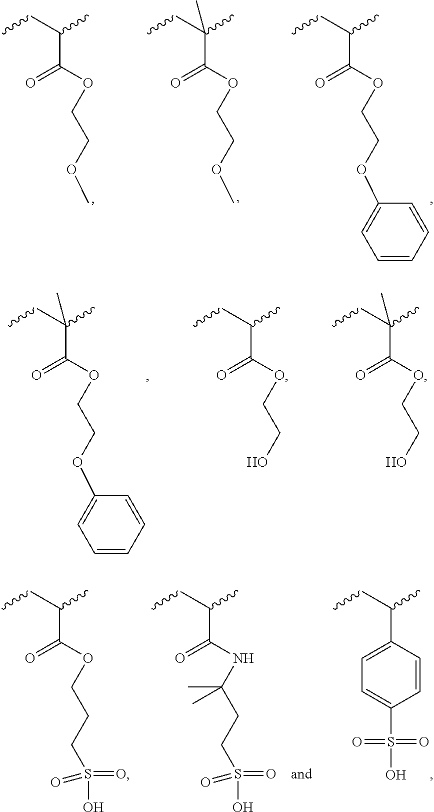
wherein n is an integer greater than or equal to 0;
R3 is selected from the following structures: 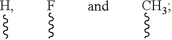
R4 is selected from the following structures: 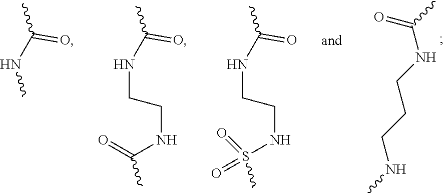
R5 or R6 or R7 or R8 is selected from the following structures: 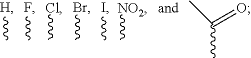
R9 is selected from the following structures: 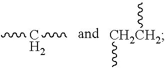
m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90);
Alternatively, the polymer has a structure represented by Formula III, 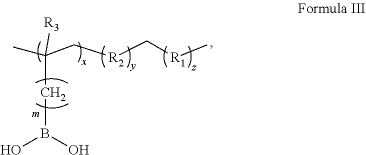
Wherein, R1 or R2 is selected from the following structures: 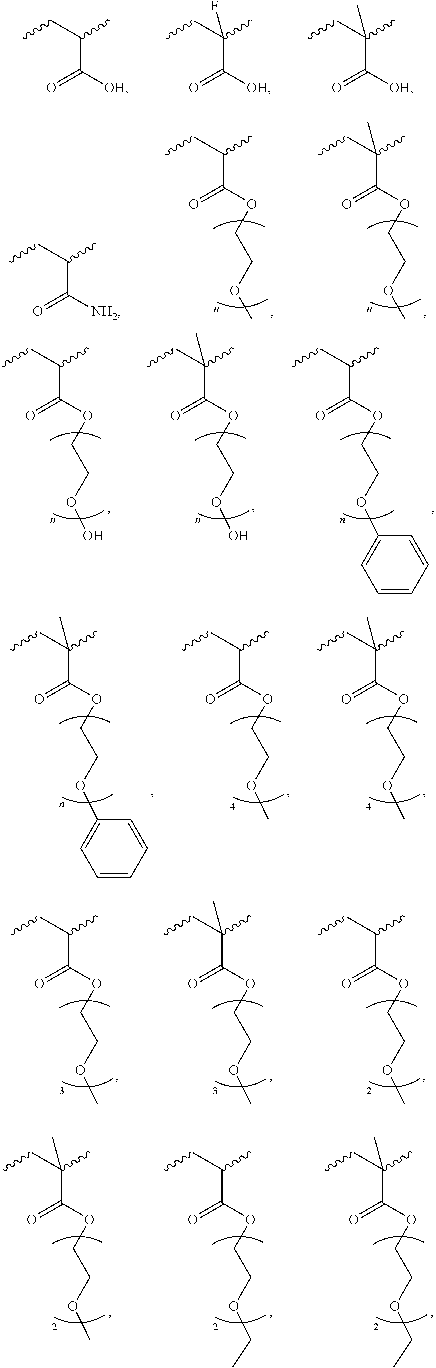 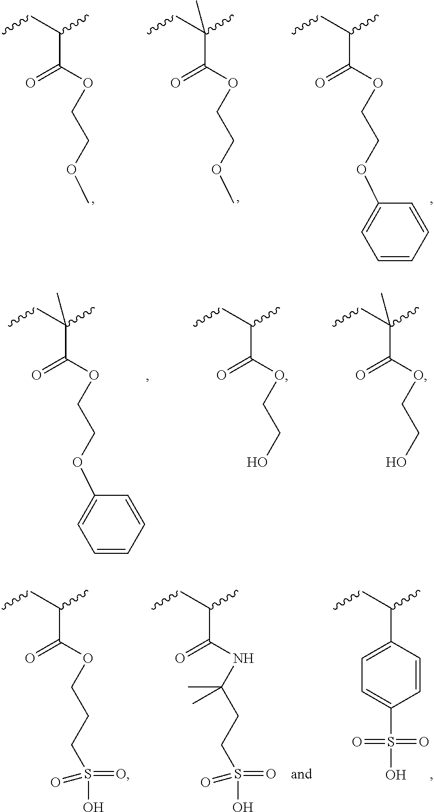
wherein n is an integer greater than or equal to 0;
R3 is selected from the following structures: 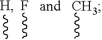
m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90).

17. The method according to claim 1, wherein the polymer comprising at least one boronic acid group is selected from:

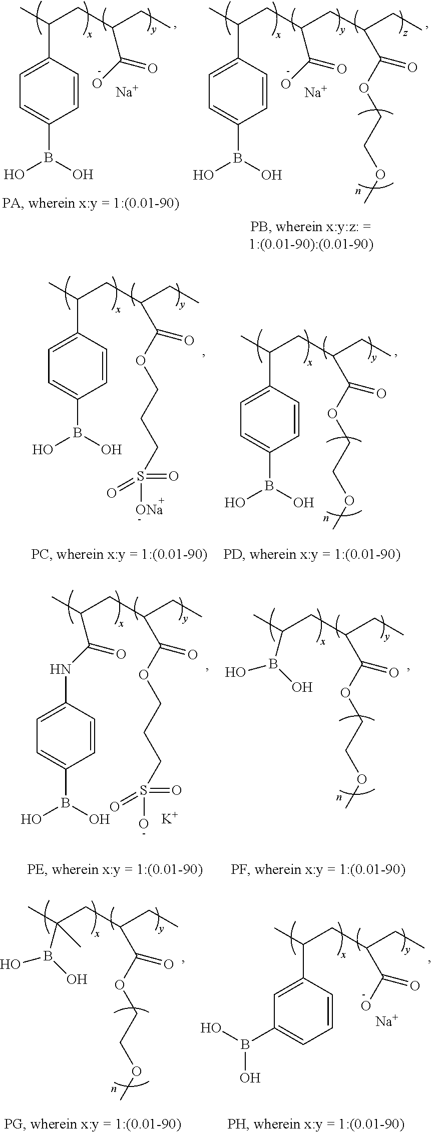

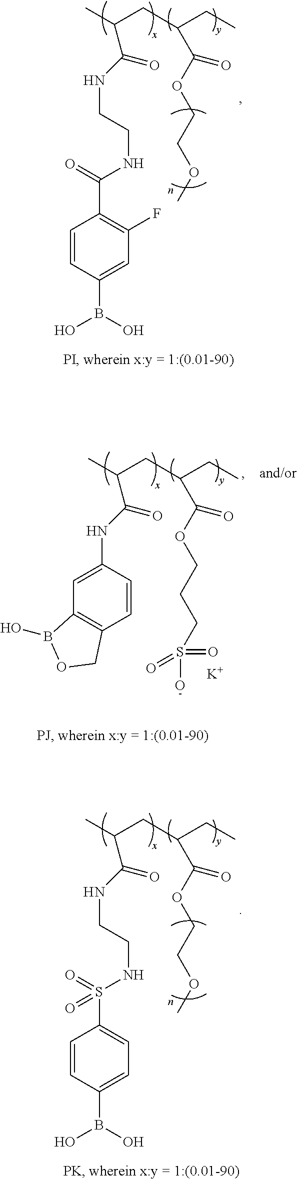

18. The method according to claim 1, wherein the polymer comprising at least one boronic acid group is selected from:

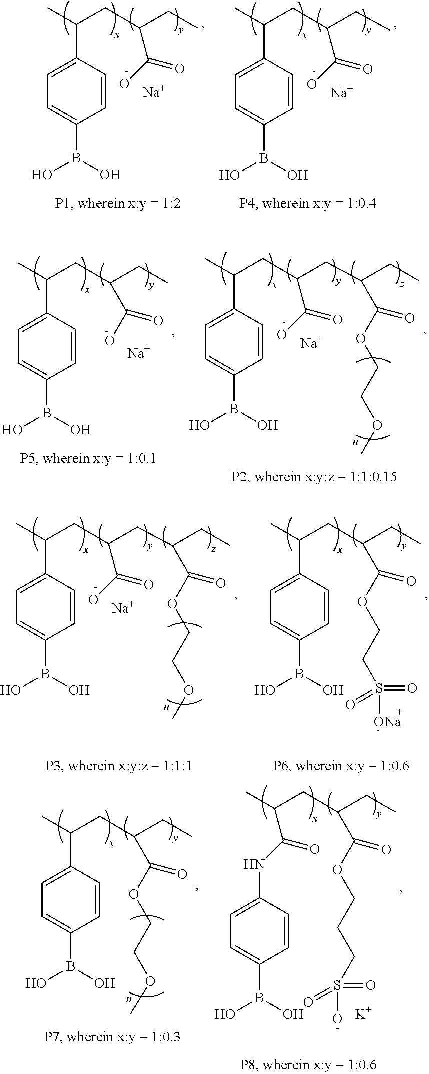

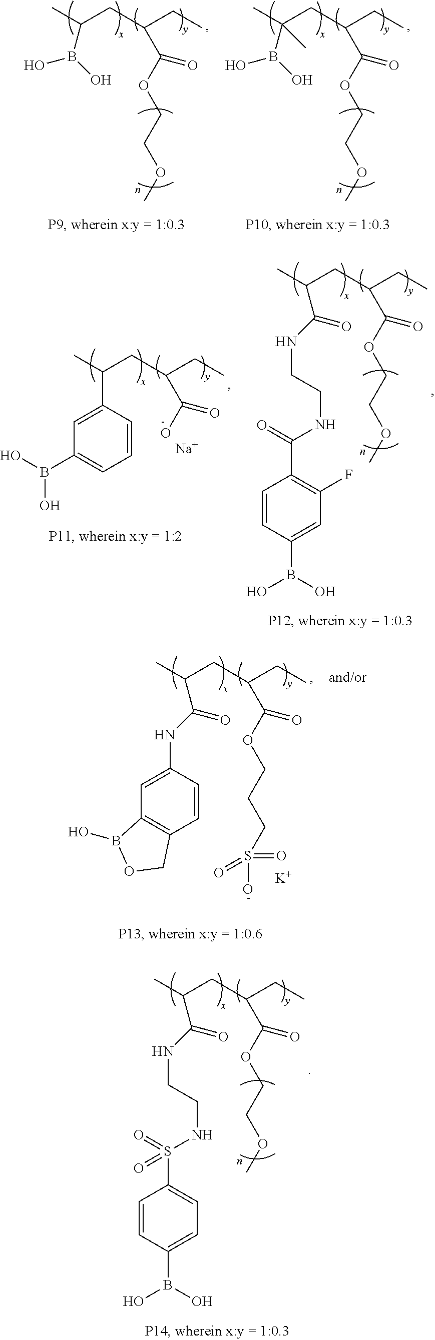

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A pharmaceutical composition, the pharmaceutically active ingredient of which comprises a polymer comprising at least one boronic acid group wherein the polymer has a structure represented by Formula I, 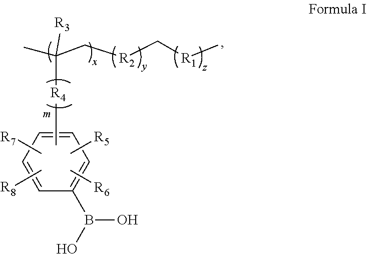
Wherein, R1 or R2 is selected from the following structures and salts thereof: 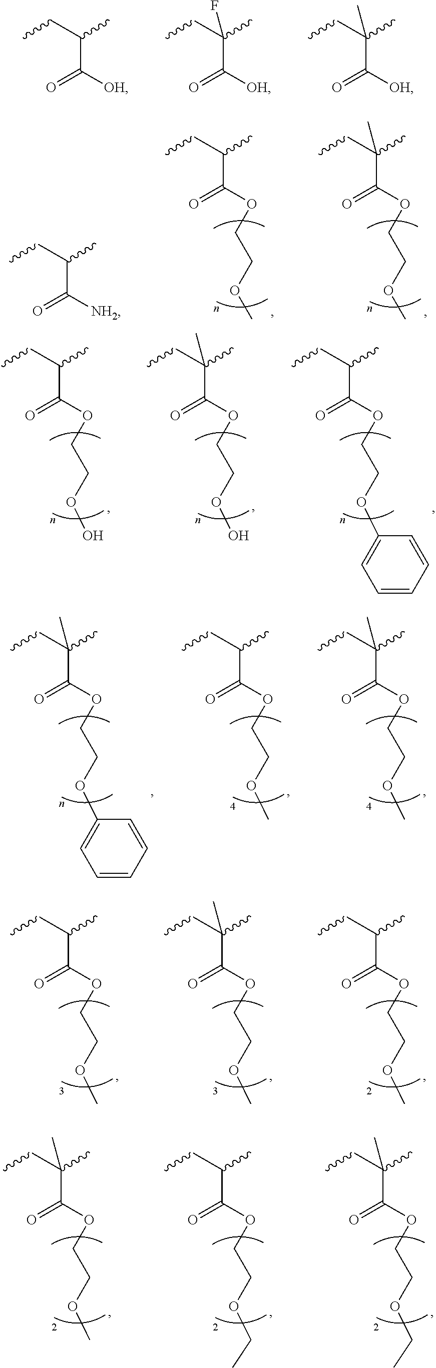 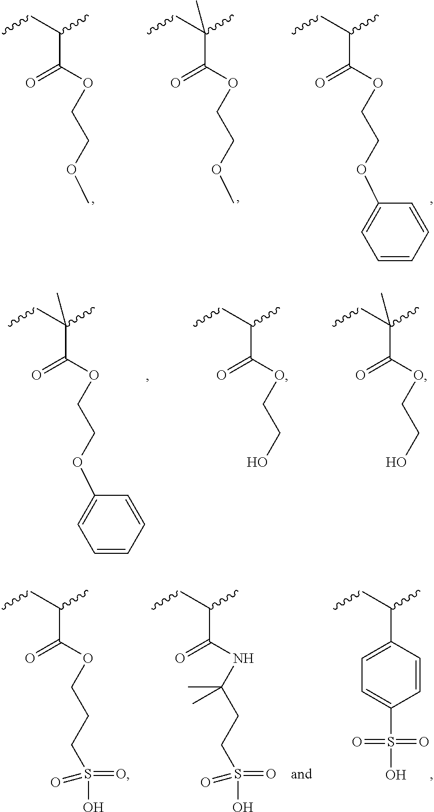
wherein n is an integer greater than or equal to 0;
R3 is selected from the following structures: 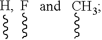
R4 is selected from the following structures: 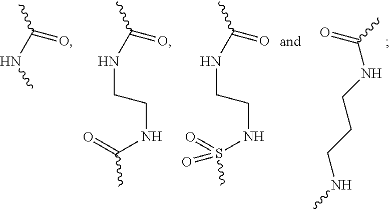
R5 or R6 or R7 or R8 is selected from the following structures: 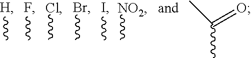
m is an integer greater than or equal to 0, x is a positive integer greater than or equal to 1, y and z are integers greater than or equal to 0, and when y is not equal to 0, x:y=1:(0.000001-90); when z is not equal to 0, x:z=1:(0.000001-90);
Alternatively, the polymer has a structure represented by Formula II, 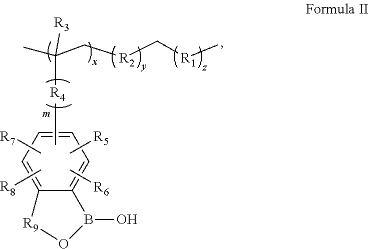
Wherein, R1 or R2 is selected from the following structures and salts thereof: 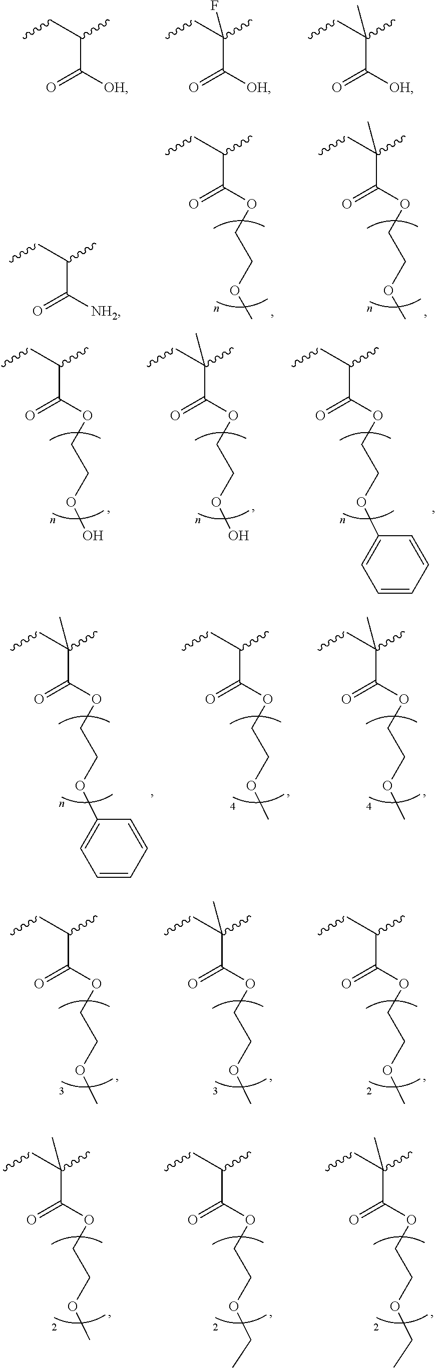 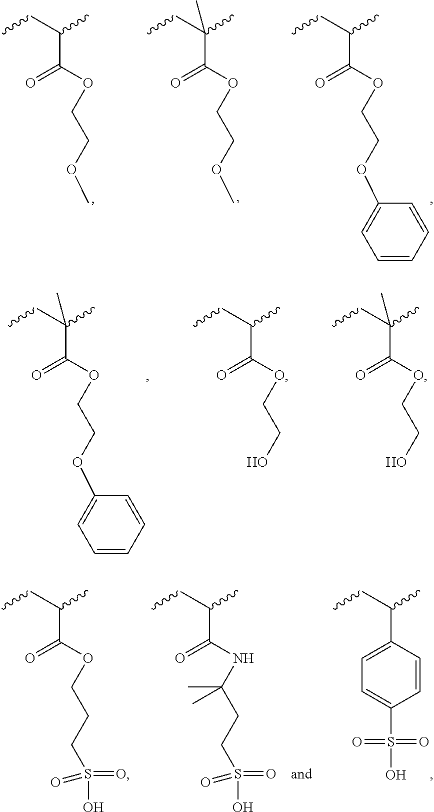
wherein n is an integer greater than or equal to 0;
R3 is selected from the following structures: 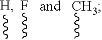
R4 is selected from the following structures: 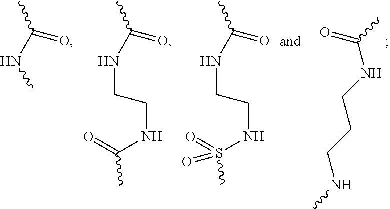
R5 or R6 or R7 or R8 is selected from the following structures: 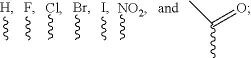
R9 is selected from the following structures: 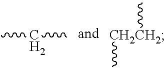
m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90);
Alternatively, the polymer has a structure represented by Formula III, 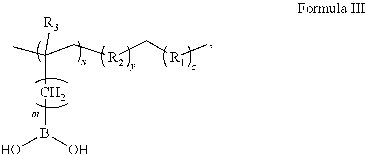
Wherein, R1 or R2 is selected from the following structures: 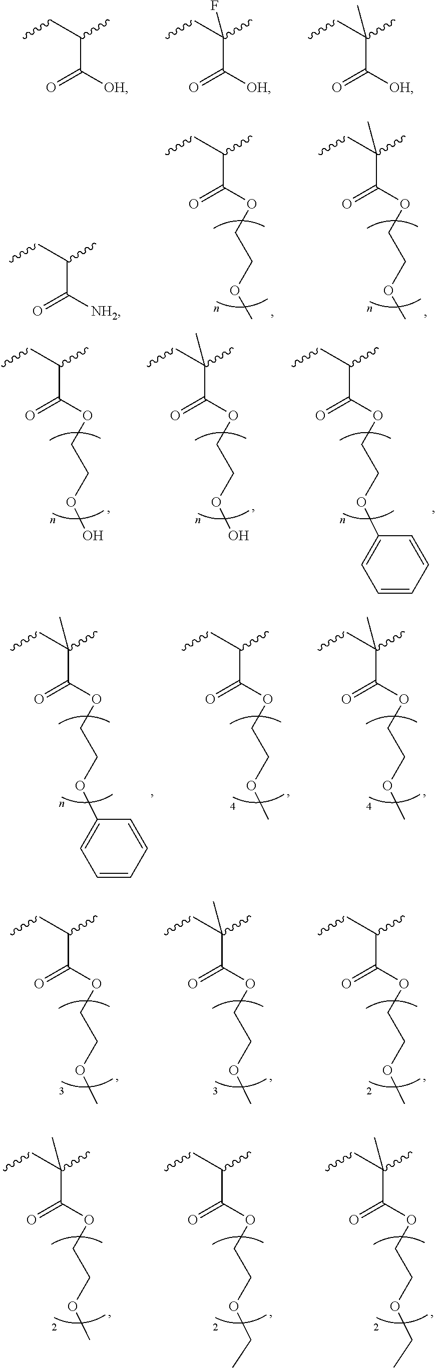 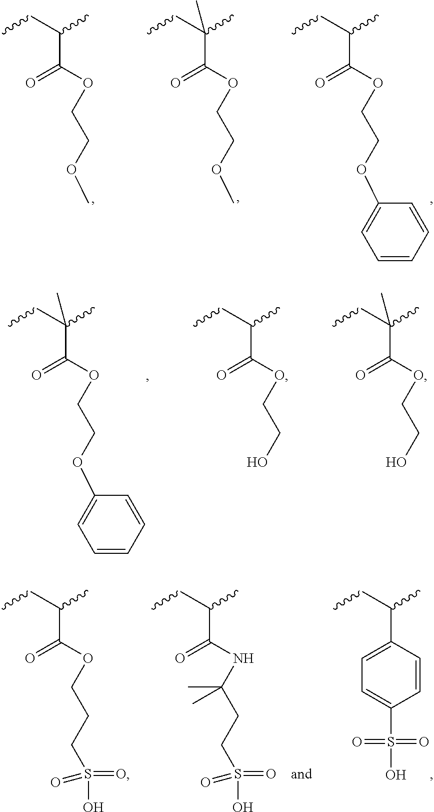
wherein n is an integer greater than or equal to 0;
R3 is selected from the following structures: 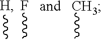
m is an integer greater than or equal to 0, x is a positive integer greater than or equal to 1, y and z are positive integers greater than or equal to 0, and when y is not equal to 0, x:y=1:(0.000001-90); when z is not equal to 0, x:z=1:(0.000001-90).

32. The pharmaceutical composition according to claim 31, wherein the polymer comprising at least one boronic acid group is selected from:

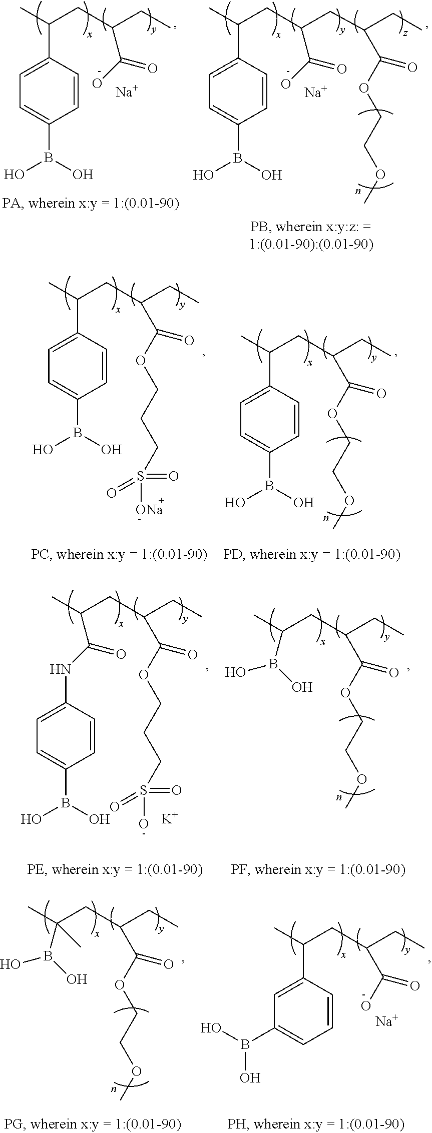

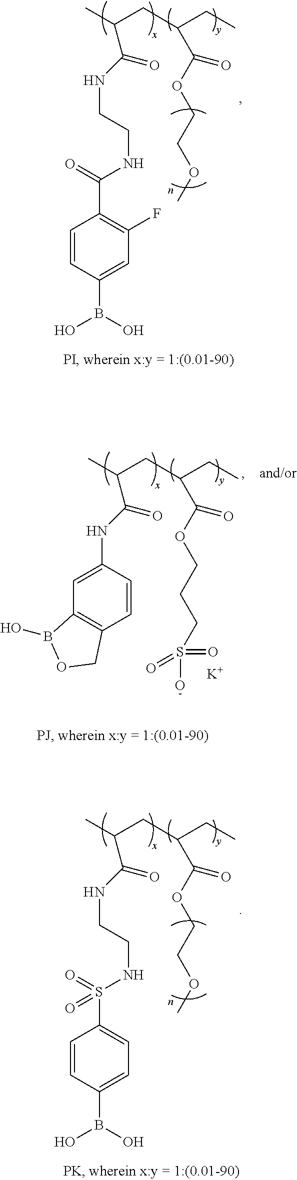

33. The pharmaceutical composition according to claim 31, wherein the polymer is selected from the following group:

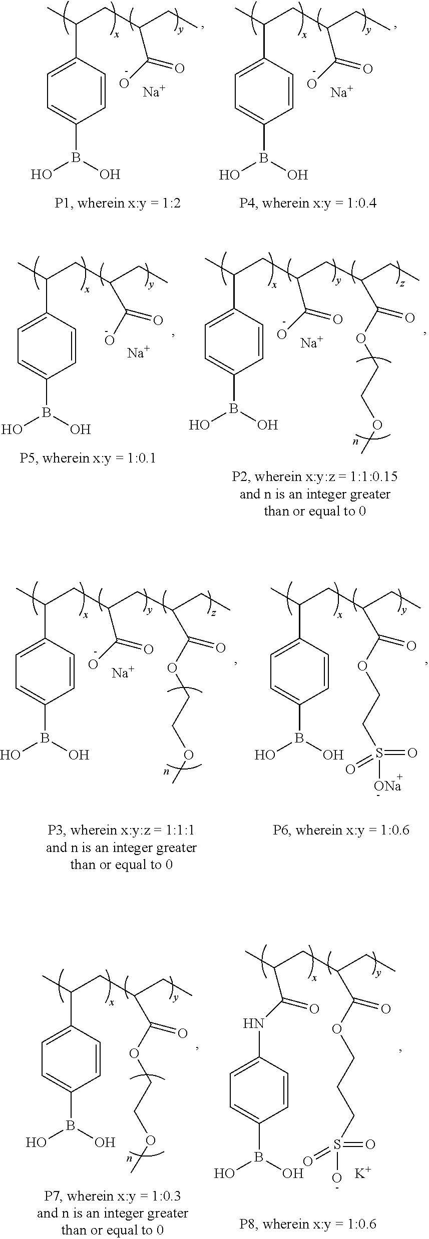

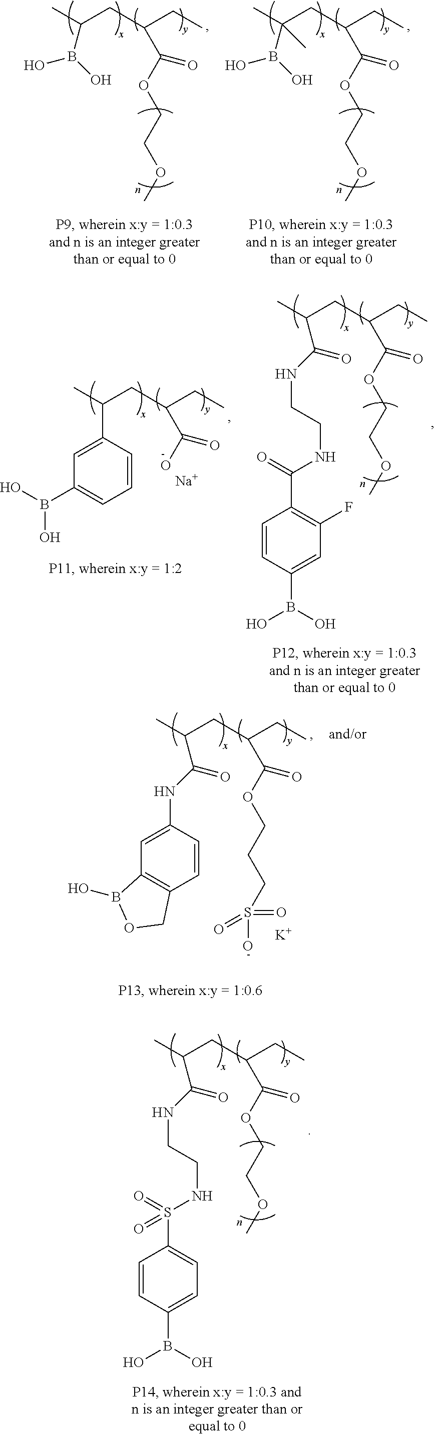

34. (canceled)

35. (canceled)

36. (canceled)

37. (canceled)

38. (canceled)

39. (canceled)

40. (canceled)

41. The pharmaceutical composition according to claim 31, which does not comprise other hypoglycemic drugs as the pharmaceutically active ingredients.

42. (canceled)

43. The pharmaceutical composition according to claim 31, which is formulated as a preparation for oral administration.
